(19) United States
(12) Patent Application Publication
(10) Pub. No.: US 2026/0257062 A1
(43) Pub. Date: Sep. 3, 2026

(54) SYSTEMS AND METHODS TO SELECT PATHWAYS FOR THE TREATMENT OF DISORDERED SLEEP BREATHING

(71) Applicant: LUNAIR MEDICAL, INC., Minneapolis, MN (US)

(72) Inventors: Orhan SOYKAN, Lino Lakes, MN (US); Mark GELFAND, New York, NY (US); Mark CHRISTOPHERSON, Shoreview, MN (US)

(21) Appl. No.: 19/552,722

(22) Filed: Feb. 27, 2026

Related U.S. Application Data
(60) Provisional application No. 63/765,321, filed on Feb. 28, 2025.

(51) Int. Cl. A61N 1/36; A61M 16/00

(52) U.S. Cl. CPC A61N 1/3601; A61M 16/0003; A61N 1/36031

(57) ABSTRACT

A system for treating sleep-disordered breathing (CSA and MSA) using an implantable device with sensors, controller, and stimulator. Electrical pulses are delivered to the phrenic nerve, optimized via feedback algorithms to enhance ventilation efficacy, reduce airway resistance, and adapt stimulation patterns for personalized therapy.

### CROSS-REFERENCE TO RELATED APPLICATIONS

This application claims priority to U.S. Provisional Application No. 63/765,321, filed Feb. 28, 2025, the entire contents being hereby incorporated by reference.

### TECHNICAL FIELD

The techniques herein relate to controlling an implantable devices that is designed to electrically stimulate the diaphragm, different nerves, and/or efferent and afferent traffic to treat sleep-disordered breathing—such as Obstructive Sleep Apnea (OSA) and Central Sleep Apnea (CSA).

### BACKGROUND

Healthy sleep enhances both physical and mental health. Sleep occurs in stages, including REM and non-REM sleep, allowing the body to rest and restore energy. Restful sleep helps to manage stress, solve problems, and recover from illness. Conversely, insufficient sleep can lead to numerous health issues, impacting cognitive and emotional well-being of an individual.

During sleep, individuals typically transition through four stages: non-REM N1, N2, N3, and REM (rapid eye movement). These stages cycle from N1 to REM and then restart with N1 or N2. Healthy children and adults typically spend approximately 50% of their sleep in N2, 20% in REM, and the remaining 30% in the other stages.

While the neurophysiology of sleep may not be completely understood, it is clear that uninterrupted, cyclic sleep, including REM stages, is essential for health. Sleep apnea syndromes disrupt this continuity, causing daytime sleepiness, fatigue, and other serious health issues. Sleep disordered breathing involves repeated episodes of apnea (cessation of breathing) or hypopnea (reduced airflow) lasting 10 seconds or more, often occurring hundreds of times per night. These apneas can be obstructive, central, or a combination of both.

Obstructive Sleep Apnea (OSA) is a well-recognized sleep disorder that affects nearly 1 billion people aged 30-65 years old, according to Benjafield et al. Obstructive sleep apnea is characterized by periodic interruptions of lung ventilation that disrupts sleep due to a momentary collapse and obstruction of the pharyngeal airway.

Obstruction of the pharyngeal airway can be attributed to decreased upper airway muscle tone and excessive relaxation of the muscles that support the soft tissues in the throat, such as the tongue and/or soft palate, and failing to maintain the airway patency. Over relaxation of these muscles results in the narrowing of the pharyngeal airway, causing airflow obstruction thus limiting airflow and the respiration, which ultimately can decrease in oxygen saturation of the tissues known as hypoxia.

Central Sleep Apnea (CSA) is a less common sleep disorder that is characterized by apneas due to a lack of signals from the respiratory center of the brain. With CSA, thoracic neural receptors fail to send the signals to the respiratory center to initiate inspiration. As a result, airflow ceases due to no respiratory muscle activity, and the same physiological condition of hypoxia results.

In CSA, the root cause lies in a dysfunction within the central nervous system, particularly in the brainstem's respiratory control centers. Consequently, during sleep, the brain fails to send the appropriate signals to the respiratory muscles, resulting in pauses in breathing or shallow breathing episodes known as apneas and hypopneas, respectively. Since the issue originates from the central nervous system's control over breathing, individuals experiencing CSA typically exhibit minimal to no respiratory effort during these apneic events. On the other hand, OSA is characterized by physical obstruction or collapse of the upper airway, often due to relaxation of throat muscles or anatomical factors. Despite the brain's efforts to initiate breathing, airflow is impeded by the obstructed airway. Consequently, individuals with OSA often demonstrate visible respiratory efforts, such as movements of the chest and abdomen, as they endeavor to overcome the obstruction and resume normal breathing patterns. Thus, the absence of respiratory effort in CSA contrasts with the presence of such effort in OSA, reflecting the differing underlying causes and mechanisms of these two types of sleep apnea.

Mixed Sleep Apnea is a form of disordered sleep breathing, formed as a combination of OSA and CSA where there is both decreased respiratory drive and decreased upper airway muscle tone, again resulting in hypoxic conditions for the patient. While the methods to open the occluded airway are utilized for the treatment of OSA, attempts to treat CSA can focus on the restoration of the respiratory drive, mostly by stimulating the phrenic nerve and the respiratory muscles, such as the diaphragm.

The upper airway is innervated by several key nerves that play crucial roles in controlling various functions such as breathing, swallowing, and vocalization. Due to their role in airway patency, these nerves have been traditionally targeted by electrical stimulation for the treatment of OSA and/or CSA.

The hypoglossal nerve is one of the key motor nerves for the movement of the tongue, controlling the genioglossus, hypoglossus, intrinsic, and styloglossus muscles. The genioglossus muscle is typically the targeted muscle in treating OSA, as it has phasic activity during inspiration and is one of the important upper airway dilator muscles. In OSA, decreased muscle tone of the genioglossus muscle causes the tongue to retract and impede airflow into the trachea. By stimulating the hypoglossal nerve, and thereby affecting the genioglossus muscle, the tongue root is pushed forward, dislodging the tongue from impeding airflow. Thus, the hypoglossal nerve is commonly targeted for the treatment of obstructive sleep apnea.

The vagus nerve is a cranial nerve that provides parasympathetic innervation to the majority of the upper respiratory tract, including the larynx and the pharynx. The vagus nerve is responsible for controlling some of the muscles involved in swallowing and vocalization, and also regulates the constriction of airway smooth muscles, secretions, and the gag reflex.

The ansa cervicalis is a loop of nerves originating from three cervical nerves (C1-C3) that innervates the infrahyoid muscles, excluding the thyrohyoid muscle. This loop of nerves consists of superior and inferior roots formed by different cervical nerves and plays a crucial role in controlling certain neck muscles. Stimulation of the ansa cervicalis and the resulting contraction of the sternothyroid muscle has been shown to pull the pharynx caudally, which increases the retropalatal cross sectional area and airflow. This, in turn, decreases measures of pharyngeal collapsibility, which can aid in the treatment of OSA.

The phrenic nerve provides complete motor innervation to the diaphragm. Phrenic afferents have been found to project into the reticular formation, where tonic drive for upper airway muscles such as the hypoglossal originate, and the rostral ventral respiratory group (rVRG) which also drives airway dilator functions. Motor innervation activation from the phrenic nerve causes the diaphragm to contract with inspiration, resulting in increased intrapleural space and a flattened diaphragm.

These cranial nerves are critically important for regulating and coordinating upper airway functions, including breathing, swallowing, and vocalization. Dysfunction of these nerves can lead to various upper airway and respiratory issues, but can also lead to a significant number of other health issues.

Sleep disorders, particularly obstructive sleep apnea (OSA) and central sleep apnea (CSA), pose significant health risks that extend beyond mere sleep disturbances. These conditions are strongly associated with various cardiovascular diseases, including hypertension, coronary artery disease, and heart failure, as well as increased risks for stroke and metabolic disorders. The impact of sleep apnea also reaches into other areas, such as heightened danger on the roads due to increased likelihood of motor vehicle crashes and the economic burden of untreated sleep apnea is substantial, affecting both individual patients and society at large.

Studies reported by Knauert et al show a clear association between OSA and hypertension, type II diabetes, stroke, coronary artery disease, and cardiac arrhythmias. Complications in the circulatory system caused by sleep apnea arise from elevated heart rates and surges in blood pressure during apneas and hypopneas. As a result, patients with OSA exhibit higher heart rates, reduced heart rate variability, increased blood pressure, and greater arterial stiffness compared to individuals without OSA, leading to the development of related cardiovascular diseases. Cardiovascular morbidities and hypertension also present themselves in patients suffering from CSA, where data from heart failure population studies suggest CSA may be present in 30% to 50% of heart failure patients.

Abbasi et al have also reported that OSA produces a chronic inflammatory state, resulting from OSA stimulating white adipose tissue. This chronic inflammatory state leads to atherosclerotic changes in the blood vessels of the patient, causing endothelial and metabolic dysfunction, further contributing to increases in cardiovascular disease. Furthermore, Hirsch et al found that the OSA severity was significantly associated with cancer risk after controlling for relevant covariates, concluding that the OSA severity is an independent risk factor for cancer.

According to Gottlieb et al., sleep disorders, particularly obstructive sleep apnea, can significantly increase the risk of auto crash risk. Gottlieb et al found that the odds ratio for any motor vehicle crash increased by 15% for every 10-unit increase in AHI in the overall population and by 17% for every 10-unit increase in AHI in participants who did not report excessive sleepiness.

Furthermore, as the severity of sleep apnea increases the odds ratio for motor vehicle crashes increased as well, showing a positive correlation. Specifically, those without sleep apnea had an odds ratio (aOR) of 0.07, indicating a 7% increase in the odds of any motor vehicle crash, those with mild sleep apnea had an increase of 13%, and those with severe sleep apnea had an increase of 123%, where the AHI of each category is AHI<5, 5 to <15, and AHI> or =30, respectively. This shows that obstructive sleep apnea is associated with motor vehicle crash risk and a danger to those suffering from this disorder.

As shown by the prior statistics, sleep disorders can adversely affect apnea patients and also have broader implications for those not suffering from the disorder. Sleep disorders increasing the risk of motor vehicle crashes is a concern for the entire public, both in terms of safety and economic impact. The American Academy of Sleep Medicine reports findings from Frost & Sullivan that details the economic impact of undiagnosed and untreated obstructive sleep apnea. These findings calculated that the annual economic burden of undiagnosed sleep apnea among U.S. adults is approximately $149.6 billion, with $86.9 billion in lost productivity, $26.2 billion in motor vehicle accidents, and $6.5 billion in workplace accidents. This amount does not include the cost for increase in health complications that result from sleep apnea, which would add an additional $30 billion annually in health care utilization and medication costs. Clearly, sleep disorders affect more than just the patient themselves.

### SUMMARY

In certain example embodiments, a device (e.g., a medical device) is provided that can be used to determine the optimal parameters for the detection of respiratory activity and the best modality for the modulation of the nervous system and/or respiratory muscles in response. Related systems and algorithms for programming the device, as well as the design parameters, are provided.

In certain embodiments, hardware and/or software is used to optimize one or more algorithms that may then be deployed and used in an implantable device. In certain example embodiments, the device can be used in an online or offline mode. In certain examples, an ongoing optimization process may be provided to take place once, periodically, or when needed.

Additional features further allow the device to work with different patients, different sensing and stimulation configurations, and with different modes of connection to the implantable device.

Other objects, features, and advantages will become apparent to those skilled in the art from the following detailed description. It is to be understood, however, that the detailed description and specific examples, while indicating some embodiments, are given by way of illustration and not limitation. Many changes and modifications within the scope of the present invention may be made without departing from the teachings of the present invention.

### BRIEF DESCRIPTION OF THE DRAWINGS

These and other features and advantages will be better and more completely understood by referring to the following detailed description of example non-limiting illustrative embodiments in conjunction with the drawings of which:

FIG. 01 shows the block diagram of the implantable system, including the stimulator, the sensors and the delivery components, such as the stimulation lead and the cuff electrode.

FIG. 02 shows a schematic diagram of the overall treatment system, including the implantable pulse generator (IPG), stimulation lead, cuff electrode, a patient programmer and the cloud connection.

FIG. 03 illustrates an example system that is used to provide stimulation to a patient according to certain example embodiments.

FIGS. 04A and 04B show different examples of time domain signals recorded for a patient with sleep apnea while the phrenic nerve is stimulated electrically.

FIG. 04C shows the changes in the airway resistance resulting from the application of the treatment in a patient, where the airway resistance was measured using two independent methods.

FIG. 05A illustrates a transthoracic impedance measurement that is accomplished using a tripolar electrode configuration, while the implanted device is stimulating the left phrenic nerve.

FIG. 05B illustrates a transthoracic impedance measurement that is accomplished using a tripolar electrode configuration, while the implanted device is stimulating the right phrenic nerve.

FIG. 05C is the electrical equivalent circuit of the configuration shown in FIGS. 05A and 05B.

FIG. 06 shows a simplified electrical block diagram of an implantable system with a nerve stimulator and a set of dual sensors, namely a transthoracic impedance sensor and a set of accelerometers.

FIG. 07 shows simultaneously recorded traces of thoracic belt, air flow, and transthoracic impedance signals from a subject during normal respiration and during central sleep apnea.

FIG. 08 illustrates an exemplary process for constructing the respiratory cycle from the transthoracic impedance (TTI) signal.

FIG. 09A shows the flowchart of the Subroutine Initialize_TTI2D.

FIG. 09B shows the flowchart of the Subroutine Run_TTI2D.

FIG. 10A is an illustration of the linear and rotational accelerations that can be measured from a patient.

FIG. 10B is an illustration of the changes in the overall gravity vector as the patient breaths.

FIG. 10C shows the results of combining gravitational acceleration measured by three accelerometers as the patient breaths.

FIG. 11 shows a diagram where the actual linear and rotational accelerations are fused to generate a combined acceleration signal.

FIG. 12A shows the flowchart of the Subroutine Initialize_Accel2D.

FIG. 12B shows the flowchart of the Subroutine Run_Accel2D.

FIG. 13 shows the 2×2 decision matrix used for classification of sleep disorders using transthoracic impedance flow and accelerometer measurements.

FIG. 14 illustrates a graph showing the impact of phrenic nerve stimulation on a patient experiencing apnea, displaying the resulting changes in respiratory flow, esophageal pressure, and tidal volume.

FIG. 15A is a graph illustrating parameters of a generic electrical stimulation pattern delivered to nervous tissue, such as the phrenic nerve, shown as a stimulation train including one or more pulses and its timing with respect to the air flow and tidal volume.

FIG. 15B illustrates the timing of the stimulation pattern applied to the phrenic nerve according to an embodiment.

FIG. 16 illustrates the timing of the stimulation pattern applied to the phrenic nerve according to an embodiment.

FIG. 17 illustrates the timing of the stimulation pattern applied to the phrenic nerve according to an embodiment.

FIGS. 18A-18B illustrate the mechanical effects of breathing. FIG. 18A shows caudal traction, while FIG. 18B presents a view of caudal traction, radial decompression, and the resulting net effect.

FIG. 19 illustrates the timing of the stimulation pattern applied to the phrenic nerve according to an embodiment.

FIG. 20 illustrates the timing of the stimulation pattern applied to the phrenic nerve according to an embodiment.

FIG. 21 illustrates the adjustments done to the timing an amplitude of the stimulation pattern applied to the phrenic nerve according to one embodiment.

FIG. 22 shows an exemplary tracing of oxygen saturation and respiratory rate as functions of time that may occur according to an embodiment.

FIG. 23 demonstrates how phrenic nerve stimulation (PNS) entrains spontaneous respiratory efforts, stabilizing airway patency and restoring ventilation, with fixed stimulation producing higher ventilation efficacy when applied with the correct stimulation rate.

FIG. 24 illustrates a periodic stimulation pattern applied to the phrenic nerve in coordination with the respiration rate to harness neuroplasticity for enhanced respiratory function.

FIG. 25 illustrates the progressive enhancement of respiratory activity with repeated phrenic nerve stimulation (PNS), demonstrating potential neuroplastic mechanisms, including short-term potentiation (STP), that improve stimulation efficacy over time.

FIG. 26 presents comparative graphs of stimulation patterns SP1 through SP6, highlighting variations in amplitude, frequency, and duration.

FIG. 27 illustrates a main mode periodic stimulation pattern applied to the phrenic nerve in the absence of sensory signals.

FIG. 28 shows stimulation burst waveform with a full length ramp up and a full-length ramp down phase.

FIG. 29 shows stimulation burst waveform with an abbreviated ramp up and an abbreviated ramp down phase.

FIG. 30 shows a set of consecutive stimulation bursts with increasing amplitude where the stimulation amplitudes start from a value near zero.

FIG. 31 shows a set of consecutive stimulation bursts with increasing amplitude where the stimulation amplitudes start from a non-zero value.

FIG. 32 illustrates clinical data showing that phrenic nerve stimulation (PNS) delivered near the onset of the endogenous respiratory effort (late expiratory/early inspiratory phase) can be effective at restoring tidal volume.

FIG. 33 illustrates the latency observed between the onset of phrenic nerve stimulation (PNS) and the initiation of inspiratory airflow.

FIG. 34 is a graph illustrating the relationship between stimulation, airflow, intrathoracic pressure, and tidal volume while the duration of the electrical stimulation is changed.

FIG. 35 is a flowchart of a procedure for iteratively adjusting parameters for the stimulation of the phrenic nerve to treat sleep apnea according to an embodiment.

FIG. 36 Illustrates a flowchart for applying and evaluating stimulation patterns to optimize ventilation efficacy, iterating through patterns until the optimal one is identified.

FIG. 37 illustrates a flowchart detailing an algorithm for selecting stimulation patterns to cycle through for treating sleep apnea, using prior collected data.

FIG. 38 illustrates a flowchart describing an algorithm for optimizing stimulation patterns by removing suboptimal options based on a phenotype to treat sleep apnea.

FIG. 39A shows an embodiment of a subroutine where the stimulation pattern is applied according to the patient's posture.

FIG. 39B shows an embodiment of a subroutine where the stimulation pattern is determined based on the patient's sleep cycle.

FIG. 39C details an algorithm that applies a default stimulation pattern initially and maintains it in the event of sensor failure.

FIG. 40 illustrates a flowchart detailing an algorithm for removing suboptimal stimulation patterns given a phenotype, where suboptimal performance is defined by multiple physiological metrics.

FIG. 41 illustrates a flowchart detailing an algorithm that chooses a stimulation pattern given the patient's current set of phenotypes based on past stimulation data.

FIG. 42 illustrates a flowchart for detecting and evaluating posture changes to determine whether to deactivate the main posture subroutine.

FIG. 43 shows the initial part of the overall algorithm according to an embodiment.

FIG. 44 shows the part of the overall algorithm for checking the reliability of sensor signals for deciding to use the default stimulation pattern without using sensors or another stimulation pattern that may use sensory feedback.

FIG. 45 shows the part of the overall algorithm for using the default stimulation in the absence of reliable sensory signals.

FIG. 46 shows the part of the overall algorithm for using the preferred stimulation pattern which may not be the default pattern.

FIG. 47 shows the part of the overall algorithm for the determination of the preferred stimulation pattern.

FIG. 48 is a graphical illustration showing the relationship between the stimulation parameters and therapy efficacy.

FIG. 49 shows the partial pressure of carbon dioxide in the blood as a function of time for the case where the loop gain is larger than one and there is no treatment.

FIG. 50 shows ventilation as a function of time for the case where the loop gain is larger than one and there is no treatment.

FIG. 51 is a block diagram that includes components of an example respiratory system model.

FIG. 52 is a graph showing the ventilatory drive as a function of partial pressure of carbon dioxide in the blood.

FIG. 53 illustrates aspects of implementation of the model predictive controller.

FIG. 54 shows a simulated metabolic rate (with CO2 generation) for a patient.

FIG. 55 illustrates simulated partial pressure of CO2 in blood for the untreated and treated (stimulated) cases as a function of time, with treatment using a model predictive controller.

FIG. 56 illustrates simulated ventilatory drive for untreated and treated (stimulated) cases as a function of time, with treatment using a model predictive controller.

FIG. 57 is a graph showing simulated simulation using a method of treatment that uses a model predictive controller.

FIG. 58-61 are graphs illustrating different aspects of simulated stimulation using a model predictive controller according to certain example embodiments.

FIG. 62A is a block diagram of a closed system formed by the stimulator/feedback controller along with the model of the respiratory controller of the patient.

FIG. 62B shows the desired change in the ventilation before (left side) and after (right side) the introduction of the feedback controller of FIG. 62A.

FIG. 63 is a graph illustrating suppression of oscillations achieved by using a model predictive controller according to certain examples.

FIG. 64 shows two parameters of used by a short demand controller according to certain example embodiments.

FIG. 65 shows respiratory characteristics of a patient using the short demand controller with a non-optimum values of its parameters.

FIGS. 66A and 66B show the parameters of the short demand controller and the effect of those parameters on mean partial pressure of carbon dioxide in blood respectively.

FIGS. 67A and 67B show the parameters of the short demand controller and the effect of those parameters on the standard deviation of the partial pressure of carbon dioxide in blood respectively.

FIGS. 68A and 68B show the parameters of the short demand controller and the effect of those parameters on mean partial pressure of carbon dioxide in blood, in tabular form, respectively, while FIG. 68C shows the standard deviation of the partial pressure of carbon dioxide in blood, in tabular form.

FIGS. 69A and 69B show the parameters of the short demand controller and the partial pressure of carbon dioxide in the blood as a function of time when there is no stimulation and when there is stimulation delivered using the short demand controller utilizing the optimum set parameters.

### DETAILED DESCRIPTION

In the following description, for purposes of explanation and non-limitation, specific details are set forth, such as particular nodes, functional entities, techniques, etc. in order to provide an understanding of the described technology. It will be apparent to one skilled in the art that other embodiments may be practiced apart from the specific details described below. In other instances, detailed descriptions of well-known methods, devices, techniques, etc. are omitted so as not to obscure the description with unnecessary detail.

Sections are used in this Detailed Description solely in order to orient the reader as to the general subject matter of each section; as will be seen below, the description of many features spans multiple sections, and headings should not be read as affecting the meaning of the description included in any section.

Unless defined otherwise, all technical and scientific terms used herein generally have the same meaning as commonly understood by one of ordinary skills in the relevant art.

The articles “a” and “an” are used herein to refer to one or to more than one (i.e., to at least one) of the grammatical object of the article. For example, “an element” means one element or more than one element.

The term “comprising” includes, but is not limited to, whatever follows the word “comprising.” Use of the term indicates the listed elements are required or mandatory but that other elements are optional and may or may not be present.

The term “consisting of” includes and is limited to whatever follows the phrase “consisting of.” The phrase indicates the limited elements are required or mandatory and that no other elements may be present.

“Sensory nerves” and “afferents” are terms used interchangeably and they refer to nerves originating at peripheral organs such as the diaphragm and carry information to the central nervous system.

“Motor nerves” and “efferents” are terms used interchangeably and they refer to nerves originating at the central nervous system and produce excitation to the muscles such as the diaphragm.

“Cuff electrode” and “electrode” are terms used interchangeably and they refer to the device that is in contact with the target tissue, such as the phrenic nerve, while being connected to the electronics.

The term electrically evoked compound action potential (eCAP) represents the synchronous firing of a population of electrically stimulated nerve fibers. It can be directly recorded on a surgically exposed nerve trunk.

A system for phrenic nerve stimulation to treat and/or cure obstructive sleep apnea, central sleep apnea, mixed apnea, and/or both central sleep apnea and mixed apnea is comprised of five major components, which are:

One or more sensors, a controller running a set of algorithms, a stimulator, implantable hardware, and external hardware.

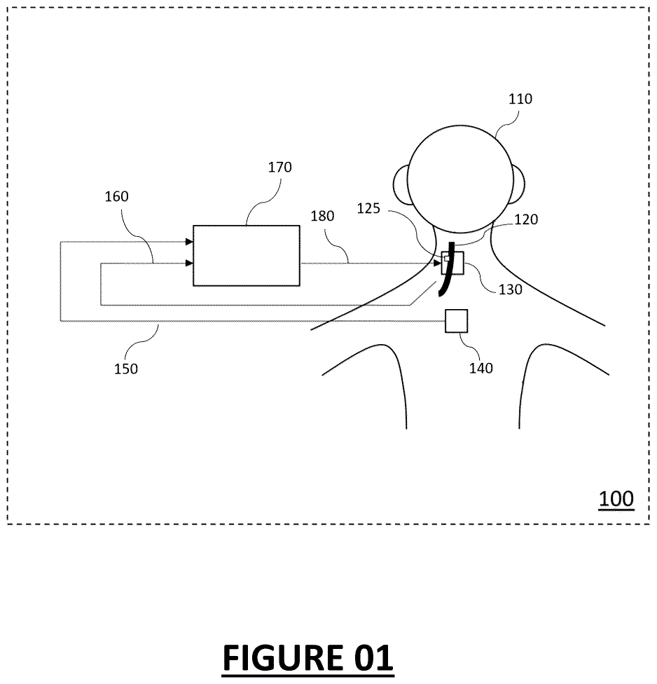

### FIGS. 1-3

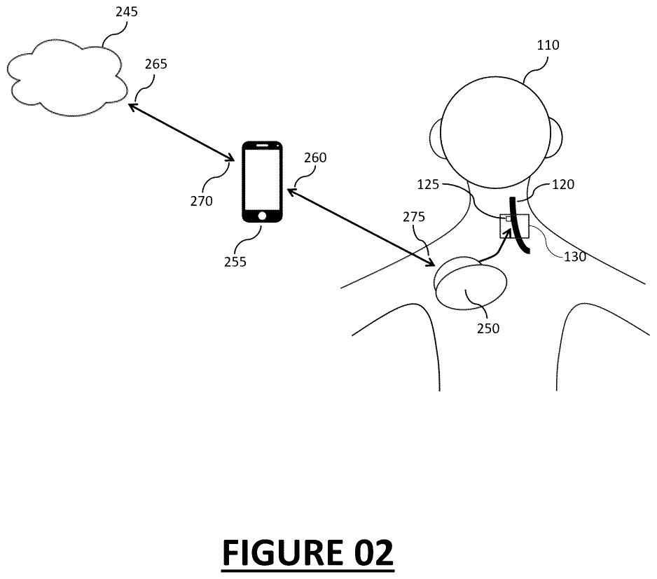

FIG. 1 shows the clinical configuration of the phrenic nerve monitoring and modulation system during its training phase, which may be during a drug induced sleep endoscopy (DISE) study. FIG. 2 shows the configuration of the implanted system. FIGS. 1 and 2 show a patient 110 and the phrenic nerve 120 of the patient 110. The phrenic nerve stimulation system 100 includes a stimulation electrode 130 that is coupled to the phrenic nerve 120 of the patient 110. In certain example embodiments, the stimulation electrode 130 (which is an example of an implantable device or implantable hardware) can be a cuff-type design.

Additional hardware includes a controller 170 that receives input signals from both external sensors 140 and internal sensors 125 located on the stimulation electrode 130. Specifically, input signal 150 corresponds to data from external sensors 140, while input signal 160 corresponds to data from internal sensors 125. These sensors may also be integrated with devices that provide electrode stimulation to the phrenic nerve 120, allowing the respective input signals (150 or 160) to be transmitted to the controller 170.

The phrenic nerve stimulation system 100 can also include one or more sensors 140 provided to sense physiological characteristics of the patient 110. Examples of different types of sensors that may be used in certain example embodiments include transthoracic impedance, accelerometer, pleural pressure, oxygen saturation, microphone, strain gauge, and so on. Sensors output signals (either in digital or analog form) via a sensory signal pathway 180 to electronics 255 (discussed below). The sensory signal pathway 180 may be wired or wireless in order to allow signals from the one or more sensors 140 to be communicated to another device.

Nerve signals provide signals regarding sensory nerve firings. Such signals are provided to electronics 255.

Phrenic nerve stimulation system 100 also includes external electronics 255. Any or all of the electronics may be implanted within the patient or may be provided externally to the patient. A stimulation lead is also provided that couples the electronics to the stimulation electrode 130.

The set of sensors (e.g., one or more sensors 140 from FIG. 1) provide the signals going into the input signal processor. Illustrative examples of different types of sensors that may be used in connection with certain example embodiments include any combination of the following: 1) passive electrical sensors that detect the electromyogram (EMG), electrocardiogram (ECG), action potential, compound action potential, and electrically evoked compound action potential (eCAP), 2) active electrical sensors such as the transthoracic impedance, 3) mechanical sensors such as pressure sensors, accelerometers, gyroscopes, strain gauges, and microphones, and 4) optical sensors such as the tissue oxygen sensors and blood oxygen sensors.

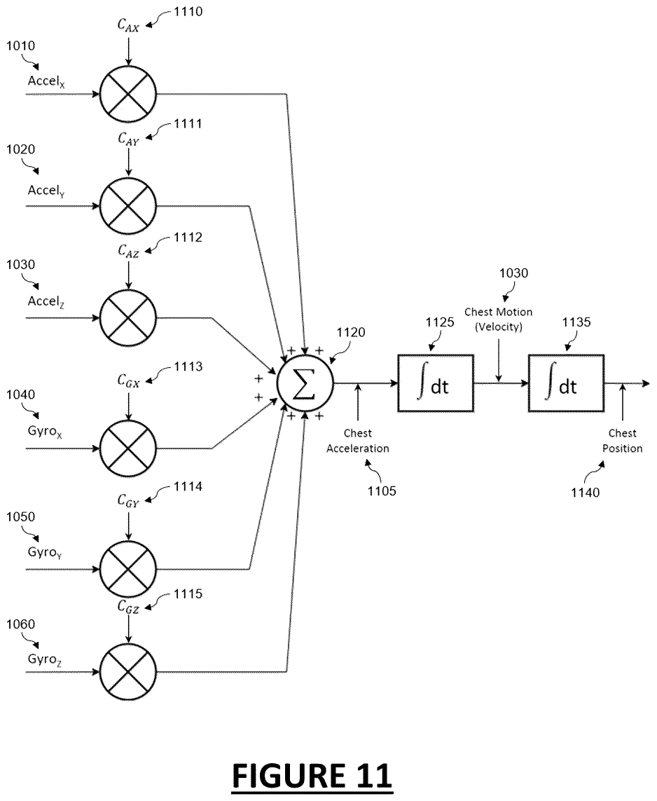

In certain example embodiments, each or any of the sensors produces a signal that is representative of the physical or physiological quantity that is being measured. For example, an acceleration sensor may produce signals that measure acceleration (e.g., proper acceleration) in 2 or 3 axes. Accelerometers may also work to detect the changes in the orientation of the implanted device by tracking the gravity vector, as shown in FIG. 11B. The signals that are produced by the sensors may be represented in certain examples as a graph or as waveforms. In certain examples, and as discussed elsewhere herein, the signals from the sensors can be analyzed and one or more values extracted therefrom (e.g., when an acceleration value exceeds a given threshold).

In certain embodiments, biomarkers of a patient can be used to assess and/or optimize treatment of sleep-disordered breathing for a patient. Example biomarkers include breath classification types, ventilation efficacy (e.g., as discussed in connection with the ventilation efficacy detector), and/or respiratory cycle data (e.g., (e.g., as discussed in connection with the respiratory cycle detector). Other data that is gathered by sensors that is based on a physical property of the patient may also be used in determining/monitoring a biomarker. Classification types can include normal breathing, obstructive sleep apnea (OSA), and/or central sleep apnea (CSA), as well as flow limited breathing and snoring. Ventilation efficacy can be measured through parameters such as inspiratory duty cycle (IDC), tidal volume (TV), peak airflow during inhalation (Φ_{MAX}), minute volume (MV), tissue oxygen saturation (Sa(O₂)), and the apnea-hypopnea index (AHI), as well as oxygen desaturation index (ODI). Respiration cycle information can include duration and/or timing of respiratory phases. Biomarkers can be used in evaluating ventilation efficiency, detecting disordered breathing events, and adjusting stimulation parameters to enhance therapeutic outcomes.

As shown in FIG. 2, information is obtained from the cloud 245 (one or more computer systems) after implanting an implantable device 130 into the patient. Following the implant, an initial set of parameters are downloaded 275 into the implant 130 via a cloud storage device 255 (a computer system that includes one or more processors). The cloud storage device 255 then can act as a bridge for collecting 270 and sending data 265 from the sensors 140 to the cloud 245. For example, the cloud storage device 255 could be a mobile phone, an Arduino, Raspberry Pi and so on, where the device connects to the cloud 245 via wired or wireless connectivity. For example, cloud storage device 255 may include ESP8266 and/or ESP32 modules that enable wireless and/or wired connectivity—which may then connect to Arduino IoT Cloud (or other third-party cloud platforms). Other cloud storage devices can include mobile (e.g., smart) phones, tablets, laptops, desktop computers, network-attached storage (NAS) devices, and IoT (internet of things) devices, and the like.

Data that is generated from the sensors 140 and the treatment parameters are stored in the cloud storage device 255 and periodically sent 265 to the cloud 245 where additional optimization may take place. Subsequently, the treatment parameters for the algorithms are downloaded 275 and used by the implant 130. This embodiment offers two advantages: First, control/adjustment of the treatment parameters may be performed in an ongoing manner. This allows, for example, the treatment parameters to be optimized over a period of time (e.g., based on data from a given patient and/or other patients). Second, as one or more sets of treatment parameters reside in the cloud, then can then be used as the initial set of parameters for other patients (e.g., future patients). Algorithms controlling this system will be discussed herein.

The controller 170 may include a hardware processor, memory, and program instructions stored therein. The program instructions may include instructions for causing the processor to perform operations in connection with any or all of the steps associated with, as noted above, any of the algorithms discussed herein.

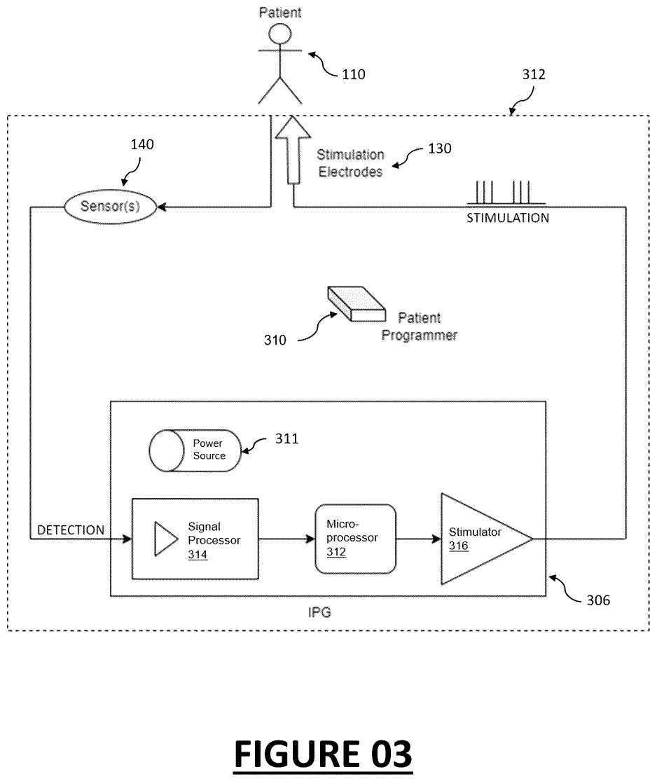

FIG. 3 is a block diagram of the controller of the phrenic nerve stimulation system 312. The phrenic nerve stimulation system 312 can include internal sensors 125 and/or external sensors 140, an implantable pulse generator (IPG) 306, electrodes 130, patient programmer 310, power source 311, microprocessor 312, signal processor 314, and/or stimulator 316. The system operates to receive signals in connection with the patient 110 and applies therapy to the patient 110. Illustrative examples of sensors 140 and electrodes 130 of the system are described above, and the following sections will describe example algorithms that may be programmed, ran, or implemented on the microprocessor 312.

It will be appreciated that the example microprocessor 312 shown in FIG. 3 is provided as part of an IPG 306 by way of example. In other examples, the microprocessor 312 and processing performed thereby may span multiple hardware processors that operate to carry out one or more steps of any or all of the algorithmic processes described herein. As an illustrative example, an example IPG may include one or more processors performing some processing and that processor may be in communication (e.g., wireless communication) with another processor that is outside the IPG. In certain example embodiments, the microprocessor may include multiple separate hardware processors that are distributed from each other. For example, a first processor may be included along with an IPG, another processor may be provided via a bedside computing device, another process in a mobile device (e.g., a mobile phone), and another processor may be part of a cloud computing platform. Collectively these multiple processors may form a controller according to certain example embodiments—e.g., a distributed controller. Any or all of the processing that is performed by a controller as discussed herein may be performed by such a controller that is distributed between two or more different processors.

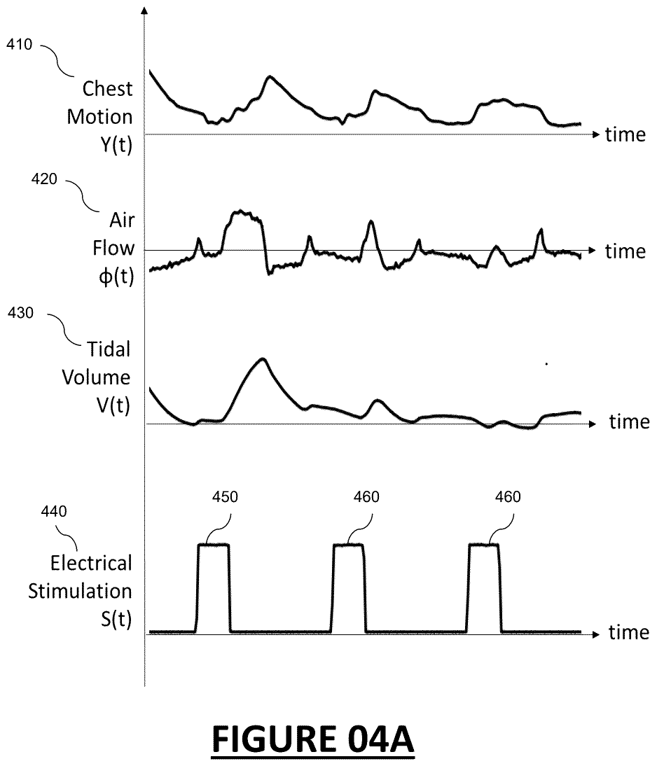

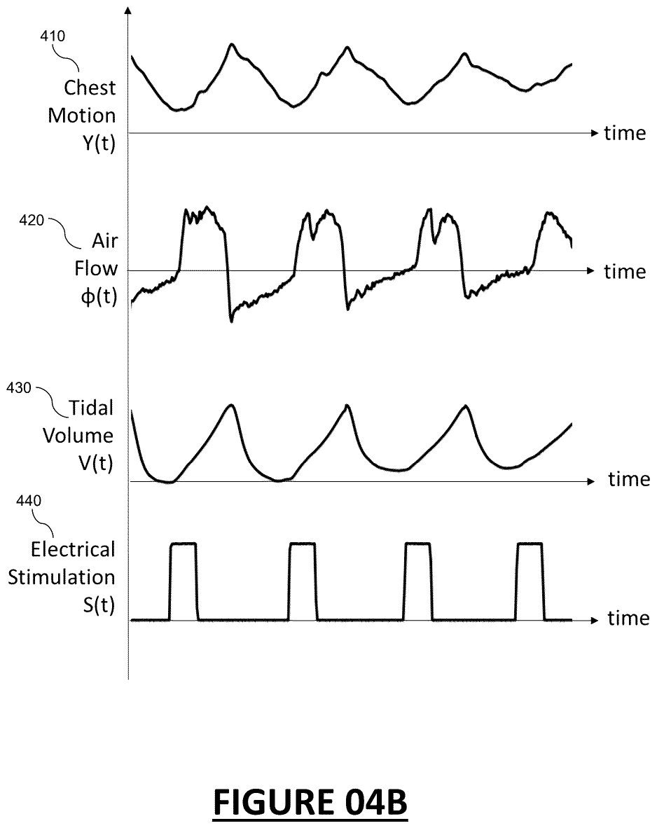

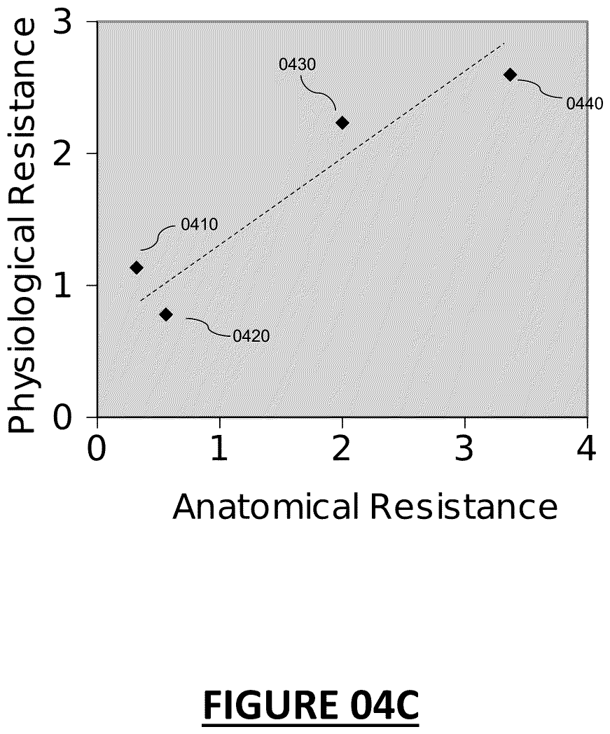

### FIGS. 4A-4C

FIG. 04A shows the recordings obtained based on a patient receiving therapy in the form of electrical stimulation 440 of a target nerve, such as the phrenic nerve. As shown in FIG. 04A, the first stimulation 450 facilitated the patient inhaling deeply (e.g., increased airflow 420 and tidal volume 430), whereas the subsequent stimuli 460 were not as effective. In certain example embodiments, the timing of the stimulations in the respiratory cycle may be a factor in determining effectiveness of stimulation to increase air into lungs. In other words, synchronizing the electrical stimulation 440 to the respiratory cycle by monitoring chest motion 410, air flow 420, and tidal volume 430 and timing the stimulation to occur at a selected portion of the cycle (phasing) can be important to the effectiveness in applying nerve stimulation signals to increase airflow into lung(s).

FIG. 04B charts a series of breaths where the stimulation (S(t)) 440 was synchronized and phased with chest motion (Y(t)) 410 to cause deep inhalations (φ(t) 420, V(t) 430). Stimulation of a nerve, such as the phrenic nerve, that results in deep inhalations are effective to treat apnea. Algorithms that utilize this information by adjusting amplitude, frequency, duration, synchronization, and tracking will be described herein.

FIG. 04C shows the results of application of electrical stimulation to the phrenic nerve of a patient. In this example, the changes in the airway resistance were calculated using both the physiological parameters and the anatomical parameters. For the calculation of the airway resistance using the physiological variables, the following equation may be used:

$$R ( t ) = \frac{Δ P ( t )}{ΔΦ ( t )} , \qquad [ Eq . 01 ]$$

where the R(t) is the time varying resistance, ΔP(t) is the time varying pressure difference across the airway, and ΔΦ(t) is the time varying value of the airflow.

For the calculation of the airway resistance using the anatomical variables, the following equation may be used:

$$\hat{R} ( t ) = {\sum }_{i = 1}^{N - 1} \frac{8 η π}{{A}_{i}^{2}} ( {L}_{i + 1} - {L}_{i} ) \qquad [ Eq . 02 ]$$

where the \hat{R} (t) is the estimate of time varying resistance, N is the number of segments of the airway that is studied, η is the viscosity of the air, A is the cross-sectional area of the airway segment, and L is the length of the airway segment.

At two instances, labelled as 0430 and 0440 in FIG. 04C, the stimulation was OFF, and the airway resistance that was calculated using both methods described above had high values, indicating that the patient was not getting enough air.

During two other instances, labelled as 0410 and 0420 in FIG. 04C, the stimulation was ON, and the airway resistance that was calculated using both methods described above had lower values, indicating that the patient was getting enough air, which indicated that the treatment that is applied in the form of electrical stimulation delivered to the phrenic nerve was effective in treating an obstructive sleep apnea.

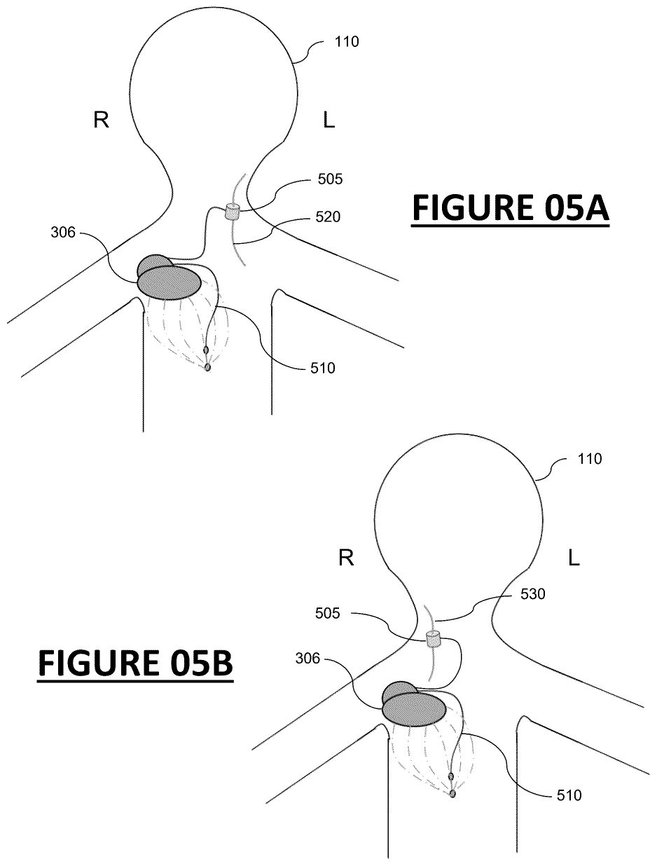

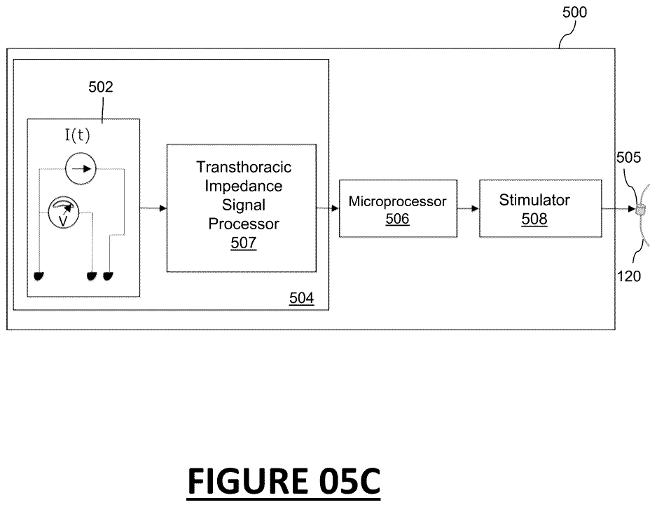

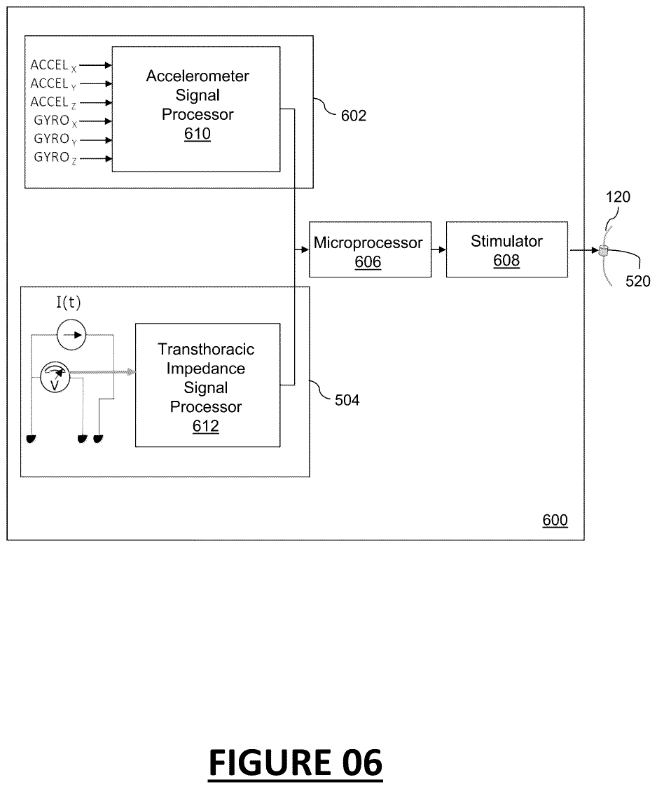

### FIGS. 5A-6: Example IPG

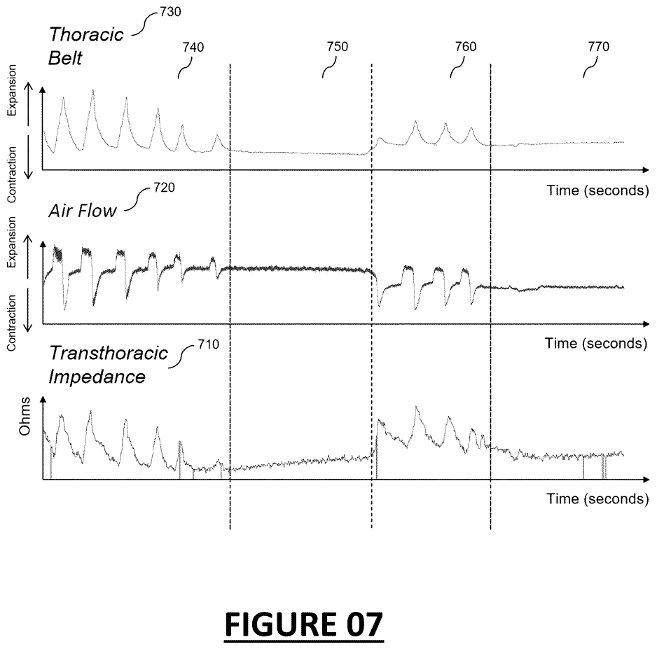

An exemplary implantable IPG 306 using a transthoracic impedance-based sensor is illustrated in FIGS. 05A and 05B, where separate stimulation 505 and sensing leads 510 are used. In these embodiments, the stimulation lead 505 applies current to the phrenic nerve 120 by way of an implantable stimulator 306. The stimulation lead 505 can stimulate the left phrenic nerve 520 or right phrenic nerve 530, shown in FIG. 05A and FIG. 05B, respectively. A high-level block diagram of the system is shown in FIG. 05C. Briefly, a tripolar transthoracic impedance measurement circuit 502 is formed using the tip and ring electrodes of the sense lead along with the implantable stimulator 505. A Transthoracic Impedance Signal Processor 507 produces an estimate of the respiratory signal. An increase in the transthoracic impedance, as shown in FIG. 07, would increase additional air presence in the lungs resulting from the inhalation of air. Conversely, a decrease in the transthoracic impedance would imply exhalation. Hence, one can use the transthoracic impedance signal to impute the phase of the respiration, e.g., increases in the transthoracic signal corresponding to the inhalation and decreases in the transthoracic signal corresponding to the exhalation. The phase of respiration can be used by the microprocessor 506 to trigger the stimulation 508 of the target nerve 120 with proper delay.

FIG. 06 is another block diagram of an implantable stimulator 600 using a pair of sensors, namely an accelerometer sensor 602 (which may be any of the accelerometer sensors described herein) and transthoracic impedance sensor 504 (which may be any of the transthoracic impedance sensors described herein).

In some examples, the microprocessor 606 receives inputs from both the Accelerometer Signal Processor 610 and the Transthoracic Impedance Signal Processor 612. The inputs are processed, and the microprocessor sends the signal to the stimulator 608 to stimulate the nerve 120 of the patient.

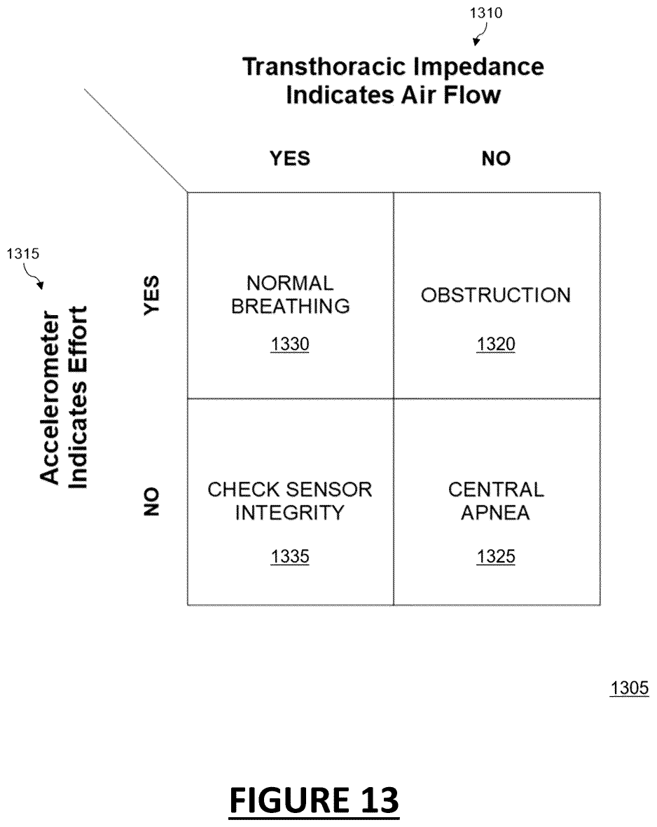

It will be appreciated that this type of implementation (e.g., with more than 1 sensor type) can provide for redundancy of sensory signals. Moreover, it may also allow the extraction of additional information from the respiratory waveforms, such as the type of sleep disorder the patient is suffering from. Additional details of such additional information is discussed in connection with FIG. 13.

### FIGS. 7-9B: Using Transthoracic Impedance

FIG. 07 illustrates how transthoracic impedance 710 can detect both normal breathing and apnea events. The thoracic belt 730 provides a signal representing the subject's breathing effort, while the airflow trace 720 reflects the resulting airflow.

As seen in the first quarter 740 and third quarter 760 of the traces in FIG. 07, the patient is actively breathing-successfully inhaling and exhaling-corresponding to fluctuations in the impedance signal. In contrast, during the second quarter 750 and fourth quarter 770, the patient experiences a central apnea, evidenced by the absence of breathing effort, lack of airflow 720, and a flat impedance signal, confirming the apnea event. Hence, the transthoracic impedance signal 710 can be used to detect the parameters of the respiratory function in real time according to certain example embodiments.

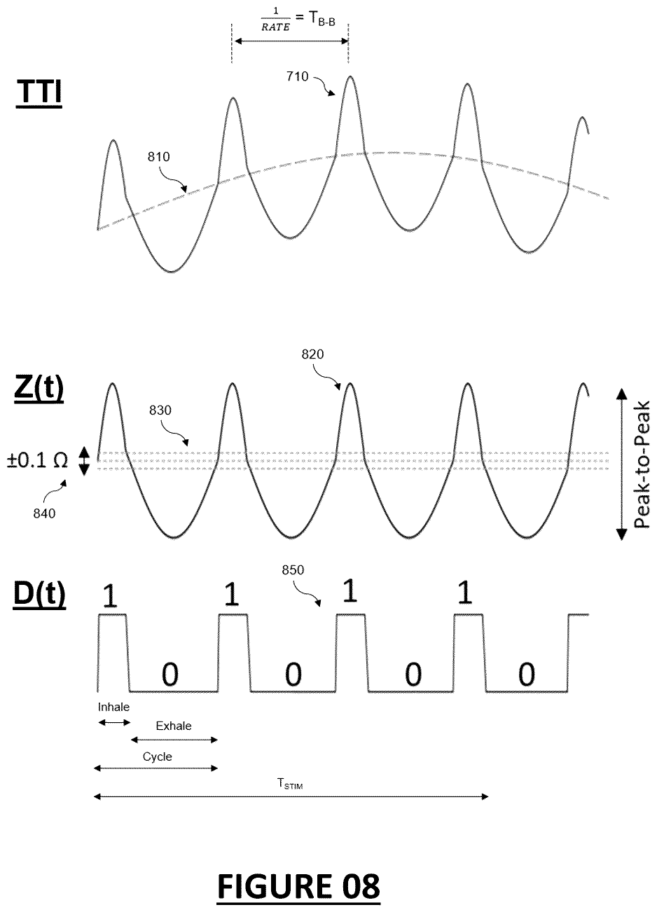

FIG. 08 shows the process that can be followed to construct the respiratory cycle from the transthoracic impedance signal (TTI) 710. Since the transthoracic impedance 710, as shown in FIG. 08, contains a drifting baseline 810, the baseline drift 810 must be removed, which can be accomplished using an equation as shown below:

$$Z ( n ) = TTI ( n ) - \frac{1}{M} {\sum }_{k = 1}^{M} TTI ( n - k ) \qquad [ Eq . 03 ]$$

where TTI(n) is the raw transthoracic impedance 710, Z(n) is the filtered impedance 820, and M is a positive integer, e.g. 50 or 750, chosen such that the duration of M samples would be longer than the expected period of the respiratory signal, i.e., 1/RATE, as shown in the top trace of FIG. 08.

It is expected that the Z(t) 820 would have a cyclic nature and will be zero-centered following the removal of the baseline drift 810, as shown in the middle trace of FIG. 08. An impedance threshold 840, such as ±0.1Ω, can be used to establish the crossing bands 830 to obtain a digital signal, D(t) 850, representing the deduced respiratory cycle signal.

Below is pseudo code of an example subroutine for the implementation of the process for conversion of TTI(t) 710 first to Z(t) 820 and then to D(t) 850:

TABLE 1

```
Subroutine Initialize_TTI2D:
      M ← M₀;
      for i ← 1 to M
      stor(i) ← 0.0;
    end for;
      D(n) ← 0;
  end Subroutine Initialize_TTI2D;
Subroutine Run_TTI2D: % for the new TTI(n)
  sum ← 0.0;
  for i ← 1 to M
      sum ← sum + stor(i);
  endfor;
  Z(n) ← TTI(n) − sum/M;
  for i ← 2 to M
      stor(i−1) ← stor(i);
  endfor;
  stor(M) ← TTI(n);
  if Z(n) > 0.1 Ω
      then D(n) ← 1;
    else
      if Z(n) < 0.1 Ω
      then D(n) ← 0;
      else D(n) ← D(n−1);
end Subroutine Run_TTI2D;
```

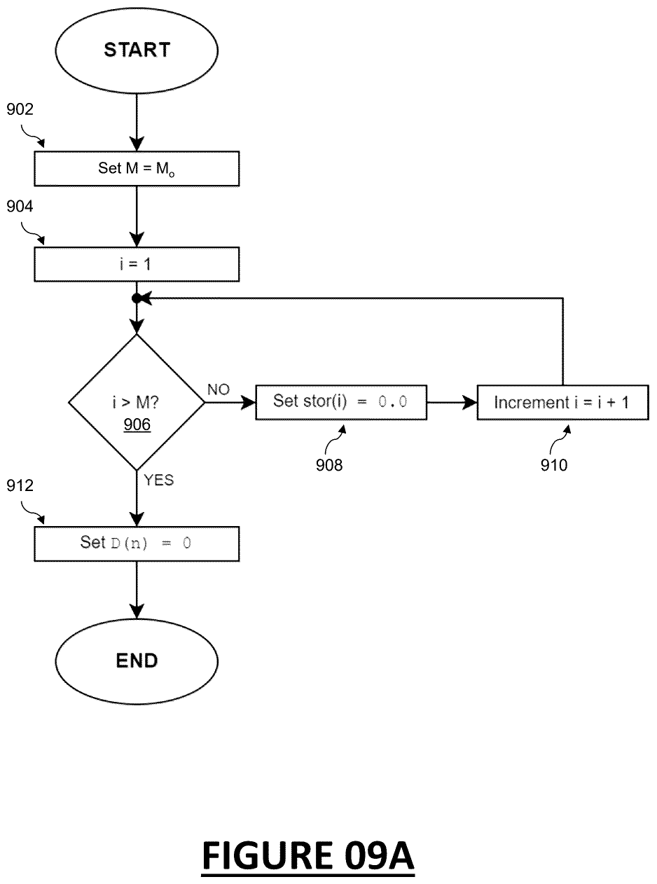

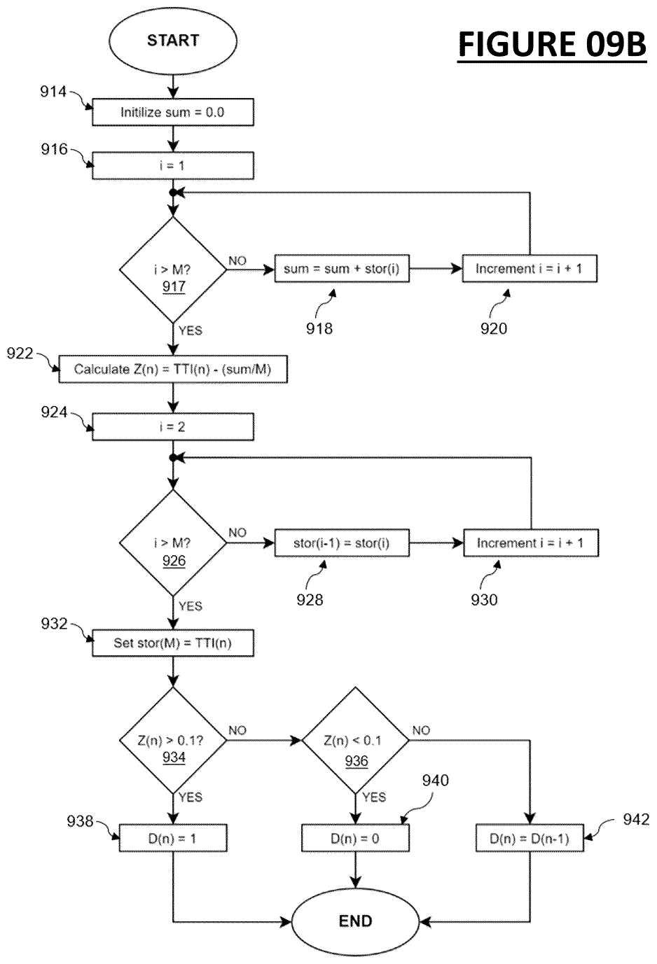

Subroutine Initialize_TTI2D is called once for the initialization of the buffer and FIG. 09A provides a flow chart of Subroutine Initialize_TTI2D. In FIG. 09A, the process begins at start, where an initial value M is set to a predefined parameter M₀ 902. The variable i is then initialized to 1 904.

A decision block evaluates whether i>M 906, determining whether the iteration should continue. If i is not greater than M, the process assigns stor(i)=0.0 908, effectively resetting or initializing storage locations. The counter i is then incremented by 1 910, and the loop continues, iterating through storage indices until i>M.

Once the condition i>M is met, D(n) is set to 0 912 and the routine is ended, finishing the initialization of the buffer.

Subroutine Run_TTI2D is called each time a new value of TTI(n) is read, and it returns a digital signal, D(t), in the form of a zero or a one, representing the deduced respiratory cycle signal. FIG. 09B contains a flow chart of the Subroutine Run_TTI2D, illustrating a method for processing transthoracic impedance (TTI) values and generating corresponding digital signals. The process begins with initialization, where the sum variable is set to 0.0 914, and an index i is initialized to 1 916.

The system iterates through stored TTI values, accumulating their sum until the index i exceeds M 917, the predetermined number of stored values. Once all values are summed, the algorithm calculates Z(n)=TTI(n)−(sum/M) 922, which represents a deviation from the averaged historical TTI values.

Next, the algorithm shifts stored values forward by assigning stor(i−1)=stor(i) 928 for all indexed values, ensuring that historical data is updated. The most recent TTI measurement is then stored as stor(M)=TTI(n) 932.

A decision-making process follows based on the computed Z(n) value:

If Z(n)>0.1 943, the system assigns D(n)=1 938, indicating a peak in TTI value.

If Z(n)<0.1 936, the system assigns D(n)=0 940, indicating a lack of TTI peak.

If neither condition is met, D(n) is set to the previous value D(n−1) 942.

This iterative method enables real-time assessment of TTI values, providing a binary digital output (1 or 0) based on detected deviations.

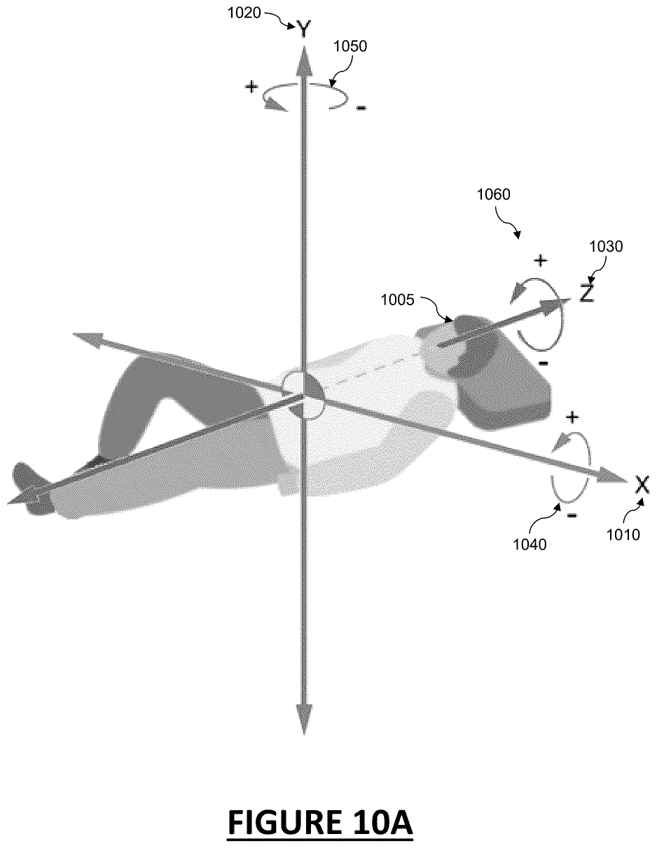

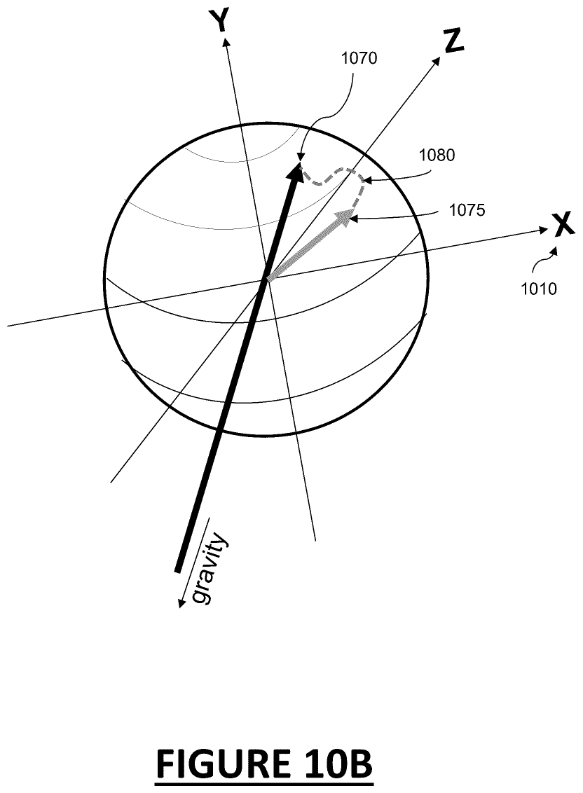

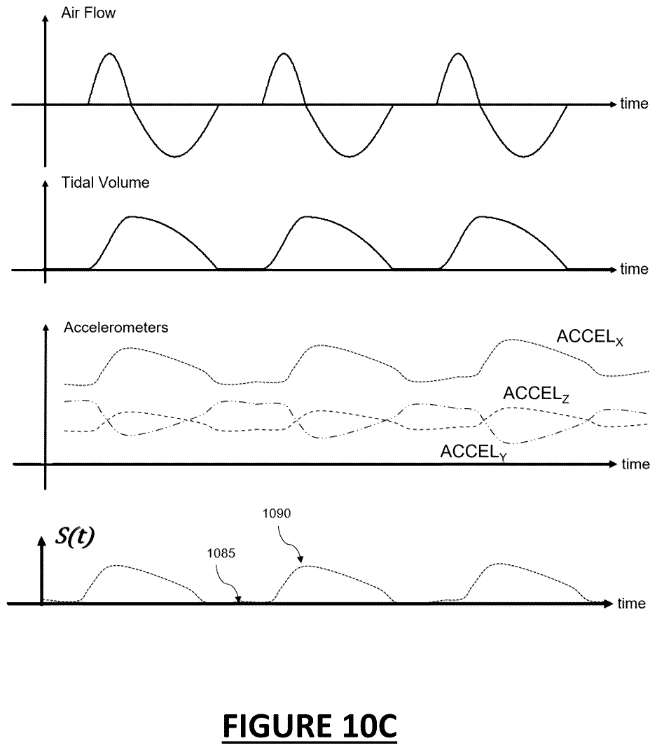

### FIG. 10A-11: Accelerometers

FIG. 10A shows the six motions of the human body 1005 that can be measured by an implantable 6-axis accelerometer, which are: 1) ACCEL_{X}: Linear acceleration in the lateral direction 1010; 2) ACCEL_{Y}: Linear acceleration in the anterior-posterior direction 1020; 3) ACCEL_{Z}: Linear acceleration in the inferior-superior direction 1030; 4) GYRO_{X} (ω_{X}): Rotational acceleration around the lateral axis 1040; 5) GYRO_{Y} (Ωy): Rotational acceleration around the anterior-posterior axis 1050; and 6) GYRO_{Z} (Ω_{Z}): Rotational acceleration around the inferior-superior axis 1060. These acceleration signals can be combined 1120 to produce a single chest acceleration signal 1105, as shown in FIG. 11.

To generate this single chest acceleration signal 1105, the individual acceleration signals are fused using patient-specific coefficients—C_{AX} 1110, C_{AY} 1111, C_{AZ} 1112, C_{GX} 1113, C_{GY} 1114, and C_{GZ} 1115—which fuse the linear and rotational acceleration components. A single integration 1125 of the chest acceleration signal 1105 can then be used to determine the chest motion 1030, with an additional integration 1135 producing the chest position 1140. Once chest motion 1130 and position 1140 is determined, ventilation efficacy can be estimated.

FIG. 10B shows the changes in the apparent projection of the Earth's gravity vector as a result of motion of the chest of the patient. In this example, the gravity vector traces a path 1080 on the unit sphere, starting from a point 1075 to another point 1070. The total distance between the points 1075 and 1070 can be calculated using the Equation 04 as shown below:

$$S ( t ) = \sqrt{{[ {A}_{x} ( t ) - {A}_{x} ( 0 ) ]}^{2} + {[ {A}_{y} ( t ) - {A}_{y} ( 0 ) ]}^{2} + {[ {A}_{z} ( t ) - {A}_{z} ( 0 ) ]}^{2}} \qquad [ Eq . 04 ]$$

where S(t) is the distance between the two points, Aₓ(t) is the acceleration in X-direction, A_{Y}(t) is the acceleration in Y-direction, and A_{z}(t) is the acceleration in Z-direction.

FIG. 10C illustrates an example of the accelerometer-based detection of the respiration utilizing the changes in the projection of the gravity vector. As illustrated in FIG. 10C, motion of the chest resulting from the respiratory function creates a time varying projection of the gravity vector on the X, Y and Z axes. The bottom trace shows the distance between the gravity projection points, 1070 and 1075 as a function of time, which is a related to the respiratory traces of Air Flow and Tidal Volume.

To implement Equation 04, it is necessary to know the non-respiratory values of the accelerometers, i.e., Aₓ(0), A_{Y}(0) and A_{Z}(0) which corresponds to the flat bottom portions 1085 of the S(t) trace in FIG. 10C. This can be done by high pass filtering of the S(t) signal and recording the value of the S(t) signal as S(0) when the high pass filtered S(t) signal has low values.

Another implementation of the Respiratory Cycle Generator relies on the use of the accelerometer data to generate the digital representation of the respiratory cycle, D(t). Pseudo code for Subroutine Initialize_Accel2D as well as Subroutine Run_Accel2D are shown below:

TABLE 2

```
Subroutine Initialize_Accel2D:
      M ← M₀;
      for i ← 1 to M
      stor(i) ← 0.0;
    end for;
      D(n) ← 0;
  end Subroutine Initialize_Accel2D;
Subroutine Run_Accel2D: % for the new accel(n)
  MAX ← maximum { stor };
  MIN ← minimum { stor };
  RANGE ← MAX − MIN;
  MID ← MIN + RANGE / 2.0;
  if accel(n) > MID
      then D(i) ← 1
      else D(i) ← 0;
for i ← 2 to M
      stor(i−1) ← stor(i);
  endfor;
stor(M) ← accel(n);
end Subroutine Run_Accel2D;
```

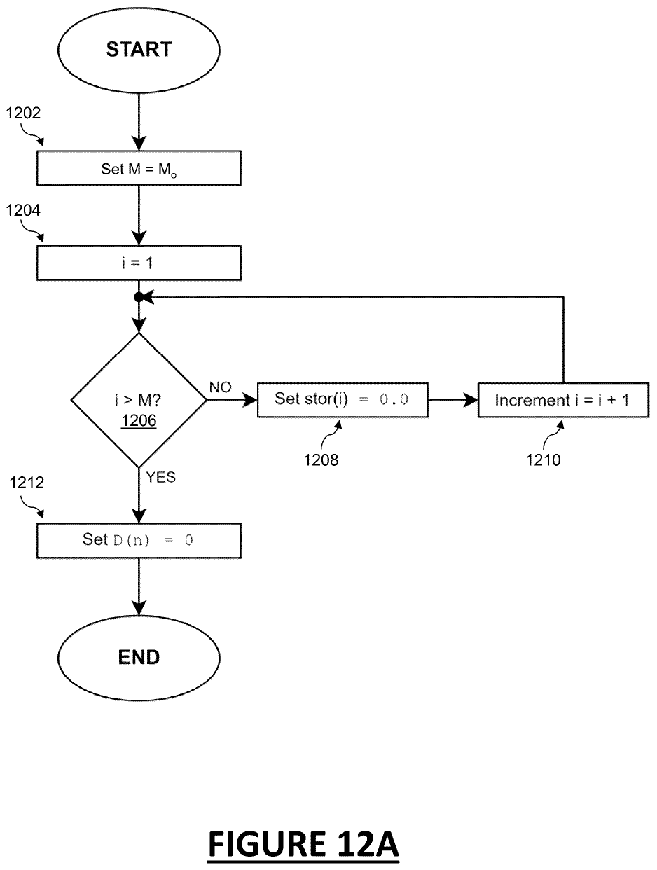

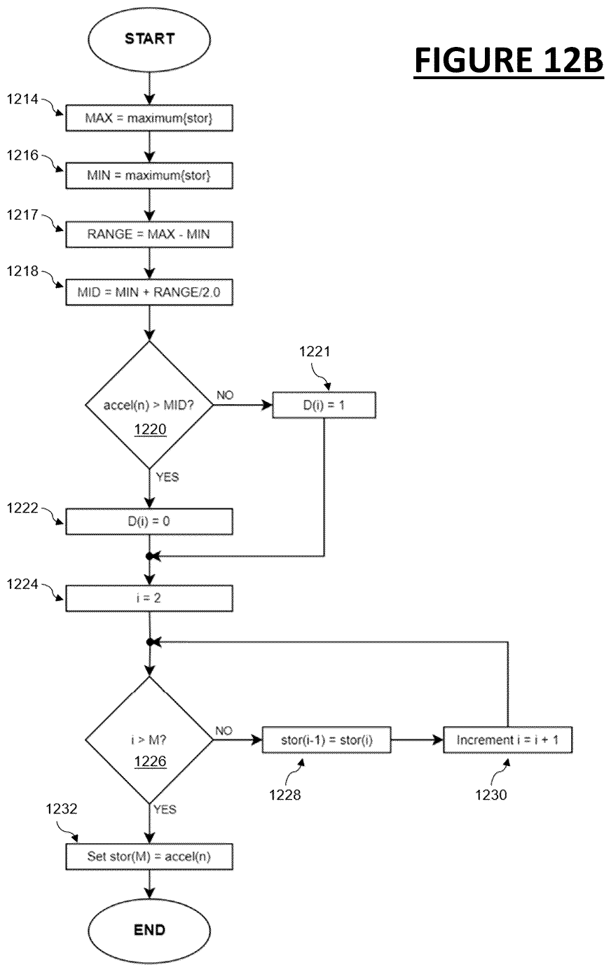

Subroutine Initialize_Accel2D is called once for the initialization of the buffer and FIG. 12A provides a flow chart of Subroutine Initialize_Accel2D. At 1202, the process begins at start, where an initial value M is set to a predefined parameter M₀. Next, at 1204, the variable i is initialized to 1.

After initialization of variables, 1206 evaluates whether i>M, determining whether the iteration should continue. If i is not greater than M, the process proceeds to block 1208 and assigns stor(i)=0.0, effectively resetting or initializing storage locations. The counter i is then incremented by 1 in block 910, and the loop continues, iterating through storage indices until i>M.

Once the condition i>M is met, the algorithm proceeds to 1212, where D(n) is set to 0 and the routine is ended, finishing the initialization of the buffer.

Subroutine Run_Accel2D is called each time a new value of accel(n) is read, and it returns a digital signal, D(t), in the form of a zero or a one, representing the deduced respiratory cycle signal. FIG. 12B contains a flow chart of the Subroutine Run_Accel2D. At 1214 and 1216, the process begins by determining the maximum and minimum values from a set of stored acceleration measurements, denoted as stor. The range is then computed 1217 and the midpoint threshold determined 1218.

Subroutine Run_Accel2D as shown above uses the threshold value that happens to be at the middle of the range from MIN to MAX, e.g., MID=(MAX+MIN)/2.0. However, it is possible to use different thresholds, by changing the threshold level from exact middle of the MIN to MAX range to a different value, such as THRESHOLD=MIN+K*(MAX−MIN), where K is a real number in the range of 0.0 to 1.0, such as 0.56. It is also possible to use a threshold value determined by non-linear means, such as THRESHOLD=median {stor}.

At 1220, the current acceleration measurement, accel(n) is then compared against the computer threshold MID. If accel(n) is greater than MID, the algorithm resorts to block 1222, where D(i) is set to 0 is accel(n) is greater than MID; otherwise, D(i) is set to 1 1221. After, at 1224, i is set to 2 and subsequently incremented if the value of i is less than M, which is defined in FIG. 12A. If i exceeds the predetermined buffer limit M, the oldest value in the storage buffer is updated with the most recent acceleration measurement 1232.

The ventilation efficacy detector can be embodied, in some examples, as a combination of hardware and software. In some examples, the functionality that is provided by the software may be instead provided by firmware or arranged hardware (e.g., an FPGA or ASIC). Ventilation efficacy can be defined as one or more of the following, including their combinations: Inspiratory duty cycle (IDC), Tidal volume (TV), Peak airflow during inhalation (Φ_{MAX}), Minute volume (MV), Oxygen Desaturation Index (ODI), Inspiratory Dity Cycle (IDC), Tissue oxygen saturation, Sa(O₂), and Apnea-Hypopnea Index (AHI).

A ventilation efficiency value is used as a measure of the quality of breathing of the patient, and the algorithms running on the processor can use this value to adjust one or more stimulation parameters. For example, the algorithms may work to maintain a minimum value of the Ventilation Efficiency, try to maximize the Ventilation Efficiency, or work toward another objective such as the maximization of the difference between the Ventilation Efficiency and the number of arousals.

There are multiple ways that the Ventilation Efficacy can be calculated, and few of them will be described below:

Inspiratory duty cycle (IDC) can be used for the estimation of ventilation efficacy. IDC is the ratio of inspiration duration to the total respiratory cycle duration, or the respiratory period. It can be estimated using the Equation 05 as shown below:

$$IDC = \frac{{T}_{D ( t ) = 1}}{{T}_{D ( t ) = 1} + {T}_{D ( t ) = 0}} \qquad [ Eq . 05 ]$$

where T_{D(t)=1} is the duration when the digital signal representing the deduced respiratory cycle signal is one, and where T_{D(t)=0} is the duration when the digital signal representing the deduced respiratory cycle signal is zero.

For the evaluation of Equation 05 above, one can use the D(t) that is produced by the subroutines of the Respiratory Cycle Generator, such as the Run_TTI2D or Run_Accel2D.

Tidal volume (TV) can be used for the estimation of ventilation efficacy. Tidal volume can be estimated as the peak-to-value of the respiration waveform that can be derived from the transthoracic impedance or accelerometer signals. For example, the Subroutine Run_Accel2D captures the peak-to-peak value of the accelerometer signal in the variable RANGE, which in turn can be used as a surrogate for the tidal volume. Alternatively, one can use the time integral or the area under the curve to estimate the ventilation efficacy.

### FIG. 13: Example Treatment Algorithms

Methods of treatment algorithms may include: 1) Mode Switching (e.g., different stimuli for OSA vs CSA and/or MSA), 2) Synchrony only (e.g., stimulate and sense to keep synchrony), and/or 3) Agnostic (stimulate all the time/some of the time, monitor retrospectively).

Methods of monitoring algorithms may include: 1) Beat to beat monitoring and immediate action, 2) Ensemble average of last N beats and react afterwards, and/or 3) Periodic monitoring and periodic response.

Methods of control algorithms may include: 1) Feedback (proportional, non-linear, PID), 2) Phase locked loop, 3) Phase/delay optimizer.

Before starting the description of the above techniques, an illustrative breath classification subroutine is introduced, as illustrated in FIG. 13. FIG. 13 shows a 2×2 matrix allowing the classification of breaths, which can be implemented as a subroutine which is described below:

TABLE 3

```
Subroutine ClassifyBreath
  if (AccelPEAK-TO-PEAK > AccelTHRESHOLD &&
TTIPEAK-TO-PEAK > TTITHRESHOLD)
    then Breath ← Normal;
  if (AccelPEAK-TO-PEAK > AccelTHRESHOLD &&
TTIPEAK-TO-PEAK < TTITHRESHOLD)
    then Breath ← OSA;
  if (AccelPEAK-TO-PEAK < AccelTHRESHOLD &&
TTIPEAK-TO-PEAK > TTITHRESHOLD)
    then Breath ← Error;
  if (AccelPEAK-TO-PEAK < AccelTHRESHOLD &&
TTIPEAK-TO-PEAK < TTITHRESHOLD)
    then Breath ← CSA;
end Subroutine ClassifyBreath;
```

FIG. 13 illustrates a logic matrix 1305 for determining if there is a disordered breathing event, and if so, which kind. The logic matrix 1305 includes whether or not there is transthoracic impedance 1310 to determine if there is an obstruction of airflow (e.g., the patient is having difficulty breathing). An accelerometer also helps to determine if there is respiratory effort 1315, which indicates whether or not the patient is experiencing obstructive sleep apnea 1320 or central sleep apnea 1325 when there is a lack of airflow. For example, if a patient has no air flow and exhibits respiratory effort, the patient is experiencing obstructive sleep apnea 1320. In another example, if a patient has no air flow but does not exhibit any respiratory effort, the patient is experiencing central sleep apnea 1325.

When transthoracic impedance indicates air flow, no type of sleep apnea is occurring, and the patient is breathing normally 1330. In the case of an accelerometer indicating respiratory effort while there is air flow, a sensor's integrity has most likely been compromised 1335, as it is unlikely for a patient to have air flow while experiencing effort.

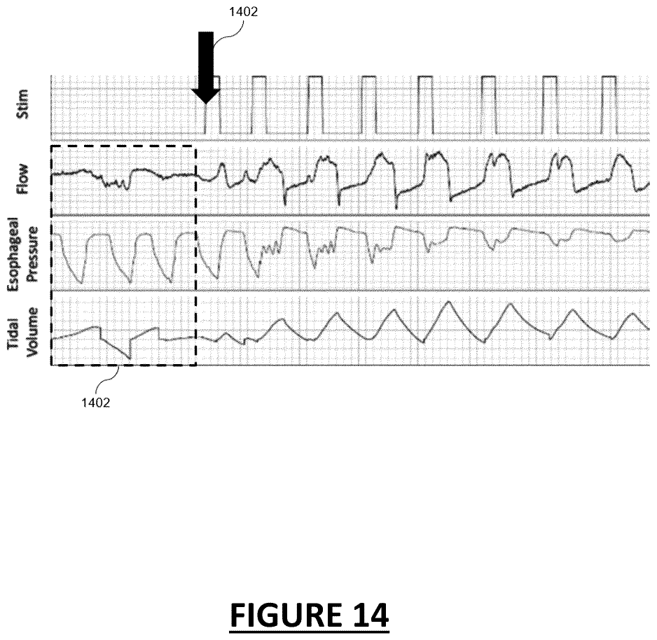

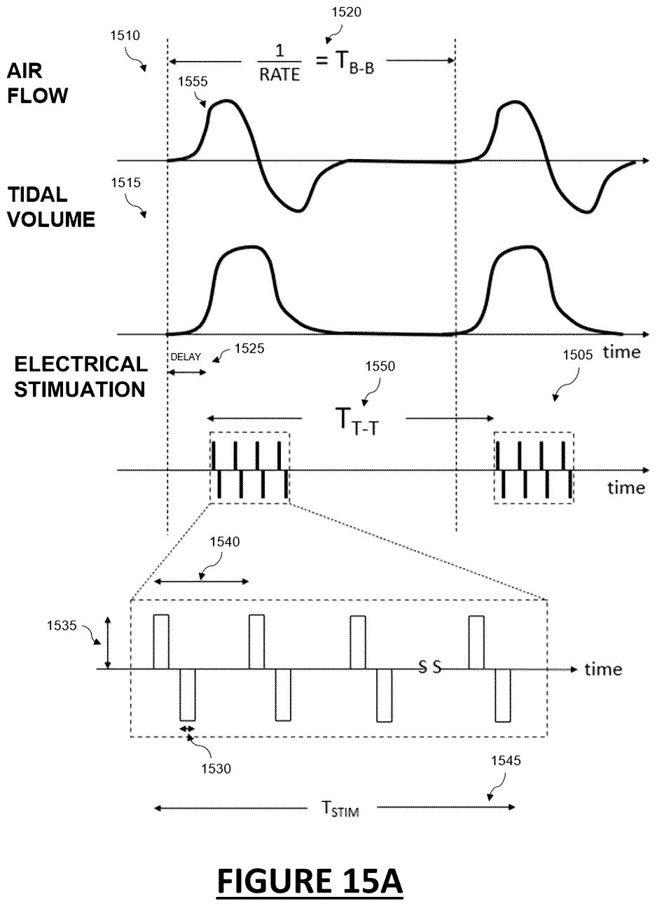

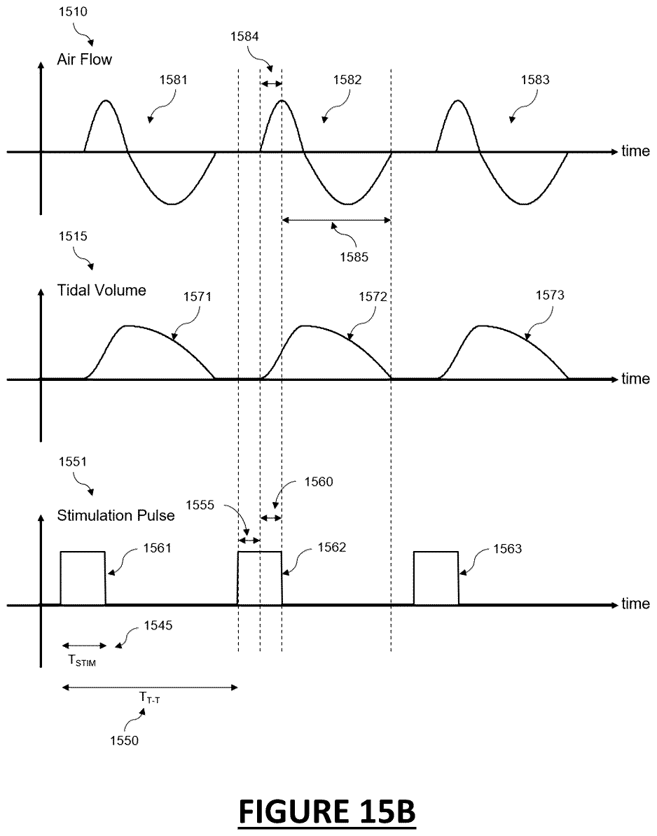

### FIGS. 14-15B: Phrenic Nerve Stimulation

FIG. 14 illustrates the effect of phrenic nerve stimulation (PNS) in alleviating obstructive sleep apnea. Prior to PNS activation 1402, the patient experiences airway obstruction 1402, resulting in the absence of airflow and tidal volume. Upon initiation of PNS, indicated by the arrow 1402, airflow and tidal volume are restored, demonstrating the therapeutic effect of stimulation. Concurrently, esophageal pressure is reduced, indicating a decrease in respiratory effort associated with airway obstruction. This figure highlights the role of PNS in mitigating obstructive sleep apnea by restoring normal respiratory function.

FIG. 15A illustrates a graphical representation of a periodic stimulation pattern 1505 applied to nervous tissue, demonstrating the correlation between the stimulation train 1505, airflow 1510, and tidal volume 1515. The stimulation train 1505 shown in FIG. 15 includes one or more pulses, as well as various parameters of the stimulation pattern described below as shown in FIGS. 08 and 15A:

T_{B-B} 1520: Respiration period or breath-to-breath interval, typically measured in seconds.

RESPIRATORY RATE: Defined as

$$\frac{1}{{T}_{B - B}} ,$$

typically in breaths per minute.

DELAY (APPROACH) 1525: Defined as t_{STIM_ONSET}−t_{RESP_ONSET}, typically measured in seconds.

PHASE: Defined as DELAY

$$\frac{DELAY}{{T}_{B - B}} ,$$

generally expressed as a percentage.

STIM STRENGTH: Comprised of three parameters:

FREQUENCY: Typically measured in Hertz (sec⁻¹), where

$$\frac{1}{FREQ}$$

is one cycle per second 1540.

PW (Pulse Width) 1530: Usually given in seconds, milliseconds, or microseconds.

AMPLITUDE 1535: Generally measured in Volts, Amperes, or milliamperes.

T_{STIM} 1545: Duration of the stimulus train, typically measured in seconds, milliseconds, or microseconds.

T_{T-T} 1550: Stimulus train period or train-to-train interval, typically in seconds.

STIMULATION RATE: Defined as

$$\frac{1}{{T}_{T - T}} ,$$

generally in breaths per minute.

DUTY CYCLE: Defined as

$$\frac{{T}_{STIM}}{{T}_{T - T}} ,$$

typically expressed as a percentage.

For a predetermined amount of time, the stimulation train 1505 is applied to the nervous tissue with some DELAY 1525 following the onset of respiration 1555, which is indicated by an increase in airflow 1510 during inhalation. Since Tidal Volume is calculated as the time integral of airflow, the Tidal Volume trace 1515 increases simultaneously as inhalation begins. The DELAY 1525 represents the time difference between the onset of the stimulation train 1525 and the beginning of the respiratory cycle, which is marked by the start of inhalation 1555. This relationship is defined in Equation 06:

$$DELAY = {t}_{STIM \_ ONSET} - {t}_{RESP \_ ONSET} \qquad [ Eq . 06 ]$$

When the DELAY value is negative—indicating that stimulation begins before the onset of the respiratory cycle—this timing is referred to as APPROACH, as defined in Equation 07:

$$APPROACH = - DELAY \qquad [ Eq . 07 ]$$

The DELAY 1525 parameter enables synchronization of the stimulation train 1505 with the patient's natural breathing cycle by timing the stimulation to occur at specific points within each respiratory cycle. This alignment enhances respiratory muscle recruitment, improves patient comfort by reducing any sensation of forced breathing, and minimizes energy consumption by activating stimulation only when necessary for effective therapy. The stimulation strength of the stimulation train has three parameters: frequency, pulse width 1530, and amplitude 1535. In FIG. 15, the stimulation train 1505 is applied in biphasic pulses, alternating the direction of current with each pulse. This biphasic pattern helps balance the charge at the electrode-tissue interface, minimizing tissue damage and preventing electrochemical buildup around the electrodes. Additionally, biphasic pulses enhance neural responsiveness by preventing nerve desensitization, thereby sustaining an effective therapeutic response over time. In other embodiments, the stimulation train 1505 may include various pulse types beyond biphasic pulses, depending on therapeutic needs and patient response. For example, the stimulation 1505 may be monophasic, delivering current in a single direction, or triphasic, with three phases to further balance charge. Burst pulses may be applied as short, grouped pulses to modulate neural response with lower energy. Asymmetric biphasic pulses, where phases differ in amplitude or duration, allow for more tailored charge delivery. Other configurations may include square-wave pulses for precise timing, sinusoidal pulses for smoother stimulation, ramp-up or tapered pulses for gradual onset and reduced discomfort, or high-frequency oscillatory pulses to selectively target specific nerve fibers.

FIG. 15B illustrates an alternative embodiment of a periodic stimulation pattern 1551 applied to nervous tissue, showing the relationship between the stimulation pulse 1551, airflow 1510, and tidal volume 1515. In this embodiment, the stimulation pulse train starts prior to the onset of inhalation 1555 and continues through the onset of inhalation 1560. Stimulating during exhalation 1582 is avoided because exhalation is a passive process, and activation at this phase could disrupt natural respiratory mechanics, interfere with physiological reflexes, and fail to induce the airway collapse characteristic of OSA, which primarily occurs during inhalation due to negative intrathoracic pressure.

Stimulating before the onset of inhalation 1555, rather than waiting until inhalation begins, enhances treatment efficacy by preemptively activating airway-supporting muscles and mitigating airway collapse before airflow is initiated. By delivering stimulation 1562 in advance, the airway can be conditioned to remain open, reducing the risk of obstruction as negative intrathoracic pressure develops at the onset of inhalation. This anticipatory approach also allows for a smoother transition into inhalation, optimizing airflow dynamics and ensuring consistent respiratory support.

Stimulation pulse 1562 can be programmed to activate a predetermined time before inhalation begins. The stimulation 1551 may be applied at a fixed interval before inhalation or for a variable period based on patient-specific needs. The duration of the stimulation pulse, T_{STIM} 1545, can be configured as either a set time interval or a percentage of the respiratory cycle. For example, if a patient's respiratory cycle is 5 seconds long, then T_{STIM} 1545 can be dynamically calculated during treatment as: T_{STIM}=(30%)×(5 sec)=1.5 sec.

In this example, the stimulation duration 1545 is 2.5 seconds, with stimulation beginning 0.5 seconds before the onset of inhalation 1555 and continuing for another full second into the inhalation phase.

In another embodiment, the stimulation duration is pre-determined to have a fixed duration, such 2 seconds. This non-adaptive approach allows the use of a simpler algorithm that does not have to adjust stimulation timing in response to variations in the length of patient's respiratory cycle. However, it should be understood that stimulation 1551 will still need to be started prior to the onset of inhalation.

The stimulation train may be tailored to adapt to the patient's specific phenotype, genetic predispositions, and lifestyle factors. To determine the optimal stimulation for each patient, a variety of stimulation patterns are provided, each designed to support specific therapeutic mechanisms of action. Below, these mechanisms of action and their corresponding stimulation patterns (stim pattern) are shown in Table 4, with their defined parameters.

TABLE 4

| | Targeted | | | Stim | | | |
|---|---|---|---|---|---|---|---|
| Stim | Mechanism of | Stim | Stim | Duration | | | |
| Pattern | Action (MoA) | Amp | Freq | (T_{STIM}) | Synch'd | Tracking | Notes |
| SP1 | Negative | Low | Medium | Short | Yes | 1-1 | |
| | Pressure | | | | | | |
| SP2 | Caudal | Medium | Medium | Long | Yes | 1-1 | |
| | Traction | | | | | | |
| SP3 | Afferent | High | High | Short | Yes | 1-1 | |
| | Stimulation | | | | | | |
| SP4 | Afferent | Very | Very | Continuous | No | N/A | |
| | Stimulation | High | Low | | | | |
| SP5 | Entrainment | Low- | Medium | Medium | Yes | 1-1 | Adjust Phase, |
| | | Med-Hi | | | | | Freq, Amp |
| SP6 | Neural | Medium | Medium | Medium | Yes | 1-q | Maximize q |
| | Plasticity | | | | | | |
| SP7 | Main Mode | Medium | Medium | Very Long | No | 1-1 | Open loop |
| | | | | (DutyCycle | | | stimulation |
| | | | | more than | | | with duty |
| | | | | 50%) | | | cycle > 50% |

Each of the above stimulation patterns are discussed in greater detail below.

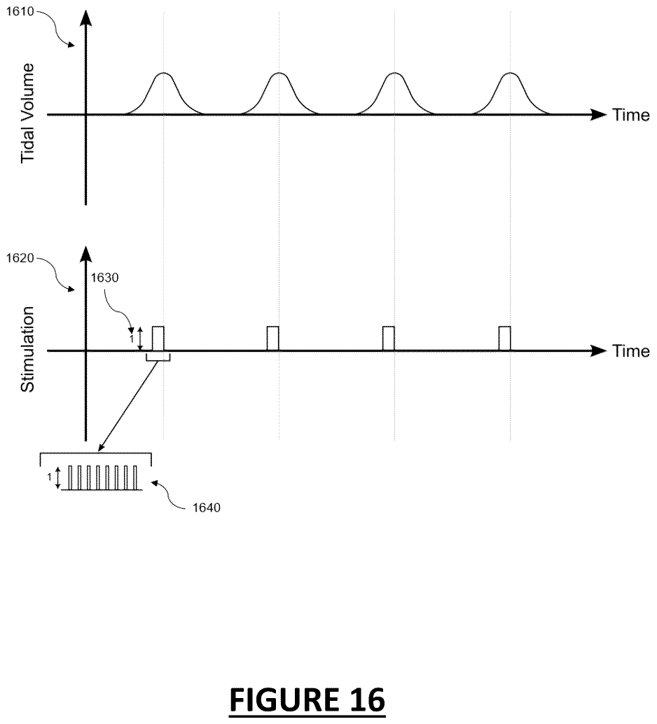

### FIG. 16: Stimulation Pattern 1

FIG. 16 illustrates the stimulation pattern SP1 1620 to specifically target negative pressure reflex. The top graph of FIG. 16 shows the tidal volume trace 1610, where the bottom graph shows the stimulation pattern SP1 1620 in synchrony with tidal volume 1610. As shown, this pattern involves a low stimulation amplitude 1630 to minimize the delivered current and prevent overstimulation, combined with a stimulation burst with a medium frequency 1640 to sustain therapeutic engagement with the targeted tissue. The duration of stimulation, or T_{STIM}, is set to a short interval to optimize the balance between efficacy and patient comfort. Additionally, SP1 1620 is synchronized with the respiratory cycle, allowing each stimulation pulse to align with the onset of natural breathing. A 1:1 synchronization ratio may be applied, where each breath cycle corresponds with a single stimulation pulse.

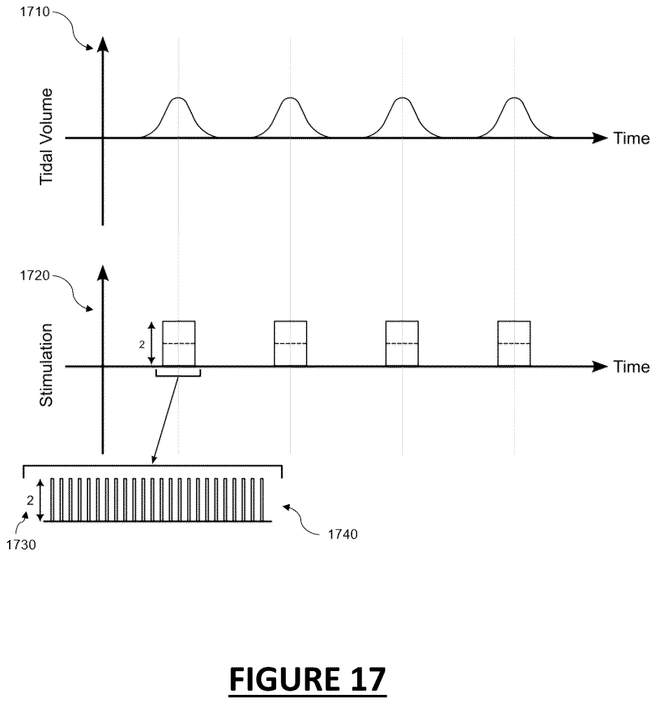

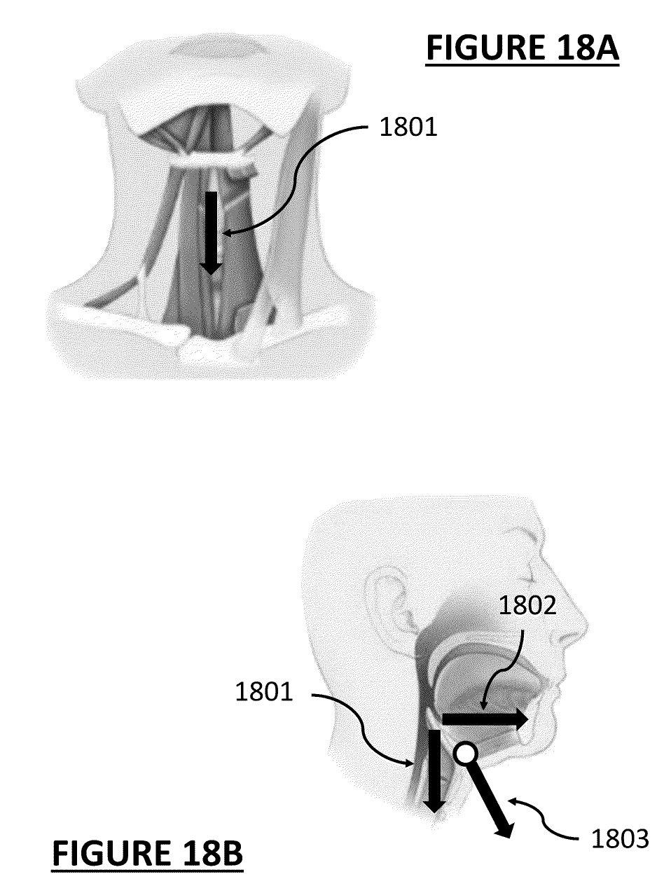

### FIG. 17-18B: Stimulation Pattern 2

FIG. 17 illustrates stimulation pattern SP2 1720, configured to target the caudal traction mechanism. This pattern applies a medium stimulation amplitude 1730 and frequency, providing sufficient current and pulse rate to effectively engage the reflex while maintaining stability over time. With a longer stimulation duration (T_{STIM}) 1740, SP2 1720 enables sustained activation of the caudal traction effect, which may support prolonged airway stabilization. Synchronized with the respiratory cycle, SP2 1720 aligns each stimulation pulse with the onset of breathing, using a 1:1 synchronization ratio to reinforce the intended traction effect in harmony with the patient's natural respiratory rhythm.

Phrenic nerve stimulation is a therapeutic technique that induces essential physiological forces responsible for breathing by directly activating the diaphragm. When the phrenic nerve is stimulated, the diaphragm contracts, creating the pressure Dongradient necessary for lung inflation and deflation, thereby simulating natural respiration. FIG. 18A-18B illustrates physiological forces that can be observed during the process of inhalation, including caudal traction, radial decompression, and net effect. The mechanical forces generated through stimulation of the phrenic nerve result in the contraction and downward movement of the diaphragm, which can pull mediastinal and cervical structures caudally, producing what is known as caudal traction.

Caudal traction can reduce pharyngeal collapsibility during sleep and anesthesia in several ways. It achieves this by applying longitudinal force vectors, enhancing mechanical connections between the thorax and upper airway structures through lung inflation, and decompressing surrounding tissues. FIG. 18A illustrates caudal traction as a downward vector 1801 resulting from the application of phrenic nerve stimulation. By applying this downward force 1801, caudal traction alters the pressure dynamics within the airway, helping to reposition structures in the throat, such as the tongue and soft palate, to prevent them from collapsing and obstructing the airway. This mechanism maintains airway patency throughout sleep. Additionally, caudal traction can stretch the pharyngeal mucosa longitudinally and stiffen the lateral pharyngeal walls. This decrease in the compliance of the upper airway offsets compressive forces from surrounding soft tissue and bony structures, further promoting airway patency. Moreover, the application of caudal traction may stimulate the muscles of the airway, enhancing muscle tone during sleep and alleviating symptoms in patients with sleep apnea.

During phrenic nerve stimulation (PNS), lung inflation not only augments the longitudinal tension but also establishes mechanical links from the thoracic cage to cervical strap muscles and the hyolaryngeal complex. While this relationship can support airway stability, it is important to recognize that the beneficial effects of lung inflation may be countered by inhibitory responses from lung stretch receptors, which can lead to increased collapsibility of the airway. Moreover, caudal traction plays a vital role in decompressing the tissues surrounding the pharynx by effectively pulling excess soft tissue away from the maxillomandibular enclosure. This decompression mimics several therapeutic strategies aimed at enlarging the pharyngeal bony enclosure or removing obstructive soft tissue, such as mandibular advancement devices and various surgical interventions like uvulopalatopharyngoplasty and hyoid advancement. Despite these approaches, their limited success highlights the need for comprehensive strategies that integrate mechanical effects like caudal traction to improve outcomes for patients with obstructive sleep apnea.

FIG. 18B illustrates additional force vectors that aid in alleviating sleep apnea by maintaining the structural integrity of the airway and preventing collapsibility, specifically radial compression 1802 and the net effect 1803. Radial compression 1802 refers to a force applied outward from the center of the body, creating pressure that stabilizes or compresses tissues in a radial direction. This force promotes stability by applying pressure to the sides of the airway, reducing the likelihood of collapse from external compressive forces, such as adipose tissue obstructing the airway. Inducing radial compression engages supportive structures in the throat, reinforcing the airway during respiration. When the forces produced by caudal traction 1801 and radial compression 1802 are combined, they create a net effect 1803 on the upper airway and surrounding structures. This net effect integrates the benefits of both caudal traction and radial compression, elongating and stiffening the upper airway while stabilizing it laterally to counteract collapse forces. Additionally, the combined forces optimize the alignment and tension of the airway tissues, leading to improved airflow and reduced incidences of obstructive sleep apnea events.

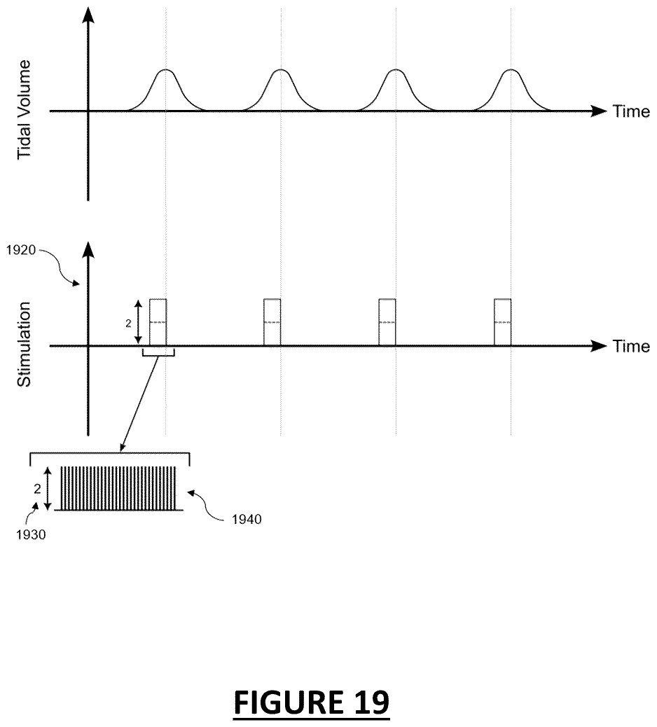

### FIG. 19: Stimulation Pattern 3

FIG. 19 illustrates stimulation pattern SP3 1920, an embodiment configured to activate afferent pathways with targeted bursts of stimulation 1940. This pattern uses high amplitude 1930 and frequency, delivering strong and rapid pulses to stimulate afferent nerve fibers effectively. With a short stimulation duration (T_{STIM}), SP3 1920 initiates quick, reflexive responses, synchronized with the respiratory cycle at a 1:1 ratio to align each pulse with inhalation, promoting coordination with the patient's natural breathing rhythm without a strong activation of the efferent pathways.

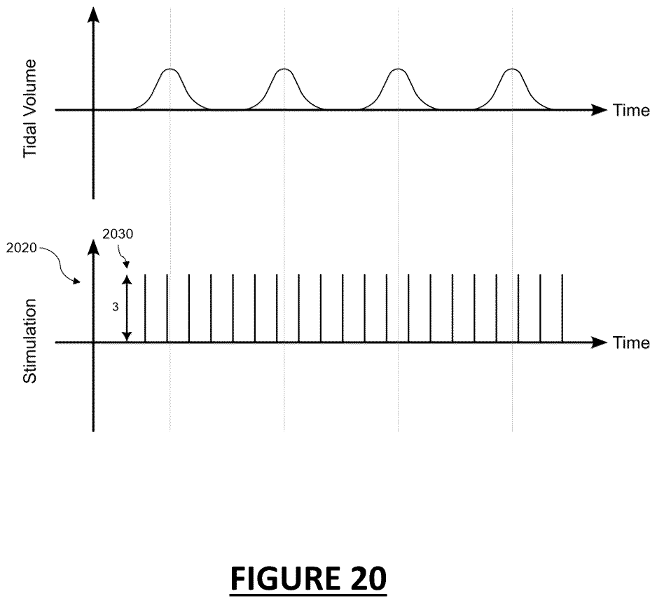

### FIG. 20: Stimulation Pattern 4

In another embodiment, shown in FIG. 20, stimulation pattern SP4 2020 provides continuous afferent stimulation. SP4 2020 employs a very high amplitude 2030 with a very low frequency, generating strong but infrequent pulses designed for sustained engagement of afferent pathways. With continuous stimulation, SP4 2020 provides an ongoing signal that is independent of the respiratory cycle, allowing for uninterrupted activation without reliance on respiratory timing.

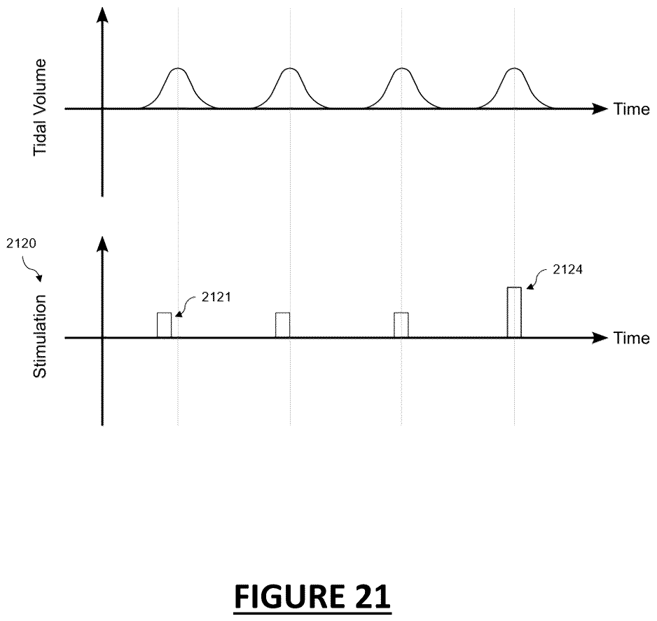

### FIGS. 21-23: Stimulation Pattern 5

FIG. 21 illustrates stimulation pattern SP5 2120, designed to achieve and maintain entrainment with adjustable parameters tailored to patient-specific needs. This pattern uses a low amplitude 2121 to a medium-high amplitude 2124 range and a medium frequency, delivering adaptable yet stable pulses to support effective entrainment. The stimulation duration (T_{STIM}) is set to medium, balancing therapeutic engagement with comfort. SP5 2120 is synchronized with the respiratory cycle, using a 1:1 ratio to align each pulse with breathing. To ensure continuous entrainment, this embodiment allows dynamic adjustment of the phase, frequency, and amplitude of stimulation, providing precise control over the entrainment response as needed.

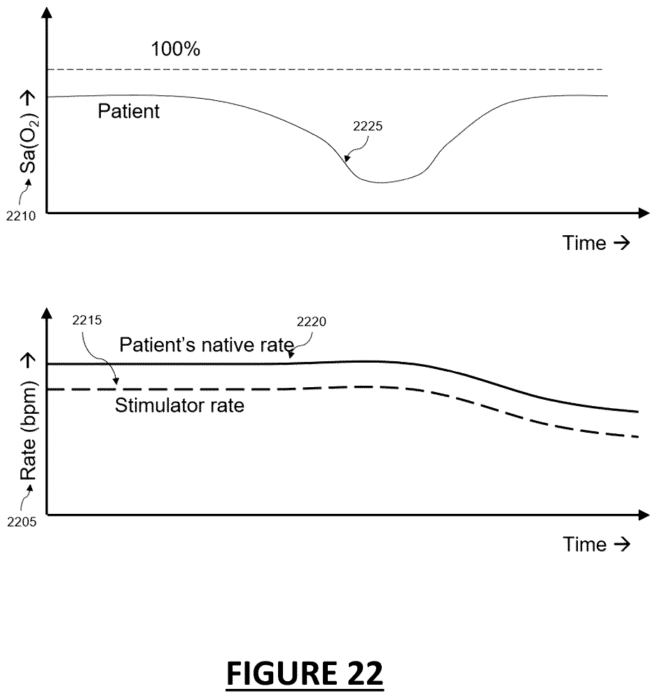

To deliver stimulation in the Entrainment mode using the stimulation pattern SP5 2120, stimulation rate that is slightly less than that of the native breathing rate of the patient may be used. For example, as shown in the graph 2205, Do if the patient's breathing rate 2220 is 16 breaths per minute (bpm), then the stimulation 2215 may be applied at the rate of 14 or 15 bpm. However, during an apnea, the patient's oxygen saturation 2210 may decrease 2225, which in turn may increase his or her demand for ventilation. Once the patient is rescued by the stimuli delivered by the implantable device, oxygen saturation would increase, and the need for ventilation along with the native respiratory rate would decrease. In order to maintain proper entrainment, it would be necessary to reduce the rate of stimulation 2215 as well, as shown in FIG. 22.

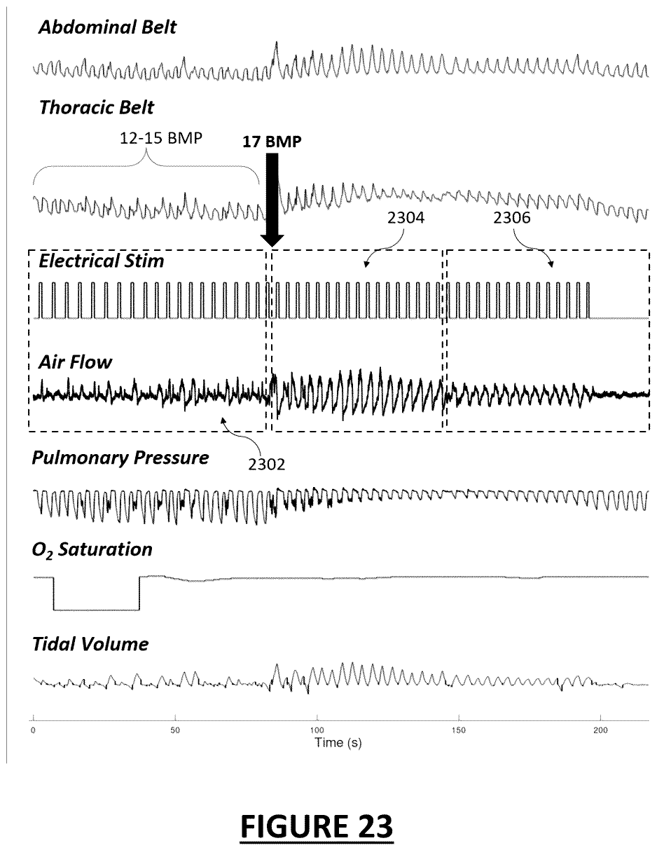

FIG. 23 shows simultaneously recorded traces of thoracic belt, abdominal belt, electrical stimulation, respiratory air flow, subglossal air pressure, peripheral oxygen saturation and tidal volume from a patient who was receiving electrical stimulation where stimulation rate was being adjusted to demonstrate effect of entrainment between the stimulation and the respiratory cycle. Clinical data shown in FIG. 23 shows that when the endogenous respiratory rhythm becomes entrained with PNS (arrow), the impact on airflow and tidal volume is entrained.

Where 2302 is not entrained.

Where 2304 is entrainment.

Where 2306 is misentrained.

With repeated application of PNS, maintenance of entrainment between the stimuli and the endogenous respiratory effort is commonly seen. In other words, with repeated stimulation, the spontaneous respiratory effort is more likely to occur in parallel with the stimulation of the phrenic nerve. This is because entrainment of the endogenous respiratory pump with appropriately timed activation of the upper airway muscles will stabilize upper airway patency and restore ventilation.

Entrainment of breathing with external signals is a well-documented physiological phenomenon. For example, during exercise, respiratory rhythms become synchronized with limb movements, a response partially mediated by the activation of somatosensory afferent pathways projecting to the medulla. Studies, such as those conducted by Potts et al using an in-situ rat model, have demonstrated that rhythmic stimulation of somatic afferents effectively entrains respiratory patterns. Similarly, exercise serves as a potent stimulus for respiratory entrainment, with breathing becoming phase-locked to limb motion.

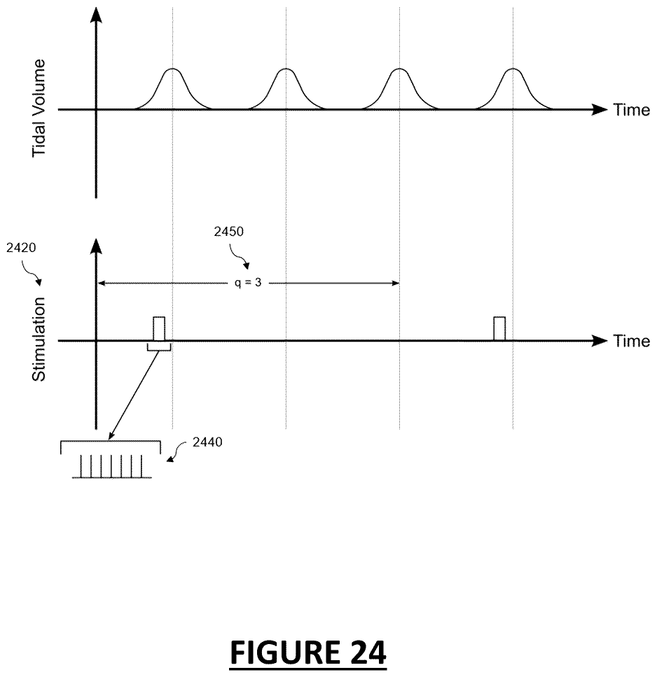

### FIG. 24: Stimulation Pattern 6

FIG. 24 illustrates stimulation pattern SP6 2420, configured to promote neural plasticity. This pattern utilizes medium amplitude, frequency 2440, and duration (T_{STIM}), creating a steady yet adaptable stimulus designed to support neural adaptation over time. SP6 2420 is synchronized with the respiratory cycle but allows for a variable 1-to-9 ratio, where q 2450 represents the number of respiratory cycles where only one of the breaths receive stimulation while the remaining breaths are not stimulated.

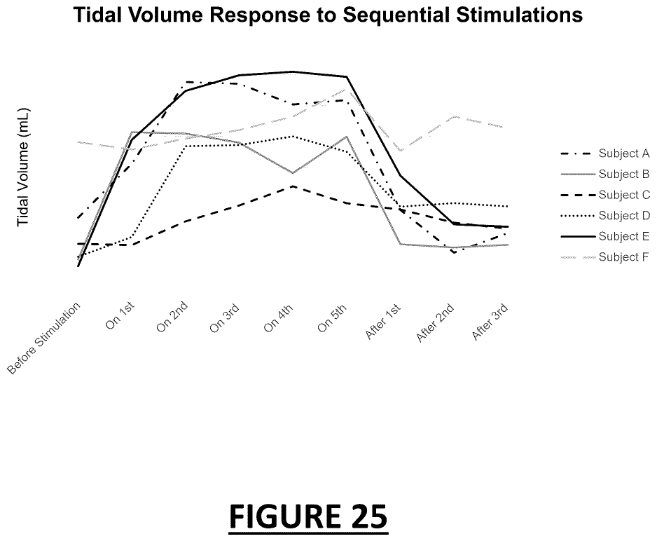

### FIG. 25:

FIG. 25 illustrates the progressive enhancement of respiratory activity associated with repeated phrenic nerve stimulation (PNS), highlighting potential neuroplastic mechanisms that enhance the efficacy of stimulation pattern 6 over time. Neurons and neuronal networks that regulate breathing in adult mammals demonstrate an ability to adapt based on experience. This plasticity may occur over seconds, hours, or days, resulting in increased or decreased respiratory-related motor output. Data collected to date suggest that the efficacy of PNS tends to improve with repeated stimulation bouts, as demonstrated in the responses shown in FIG. 25. The observed response may represent short-term potentiation (STP), a form of respiratory plasticity induced by short periods of stimulation. STP generally manifests as a progressive enhancement of respiratory activity following the onset of stimulation, followed by a gradual decline to baseline levels after the stimulation is removed. This phenomenon has been described in both upper airway motor activity and diaphragm activity.

The clinical data in FIG. 25 may reflect short-term plasticity in the neural circuits controlling breathing, specifically in response to phrenic afferent stimulation. Supporting this interpretation, Streeter et al. demonstrated that repeated episodes of phrenic nerve stimulation induced a transient increase in phrenic nerve output that persisted for approximately five minutes after the stimulation ended. Follow-up studies confirmed these findings and further established that the observed plasticity required the activation of phrenic afferents.

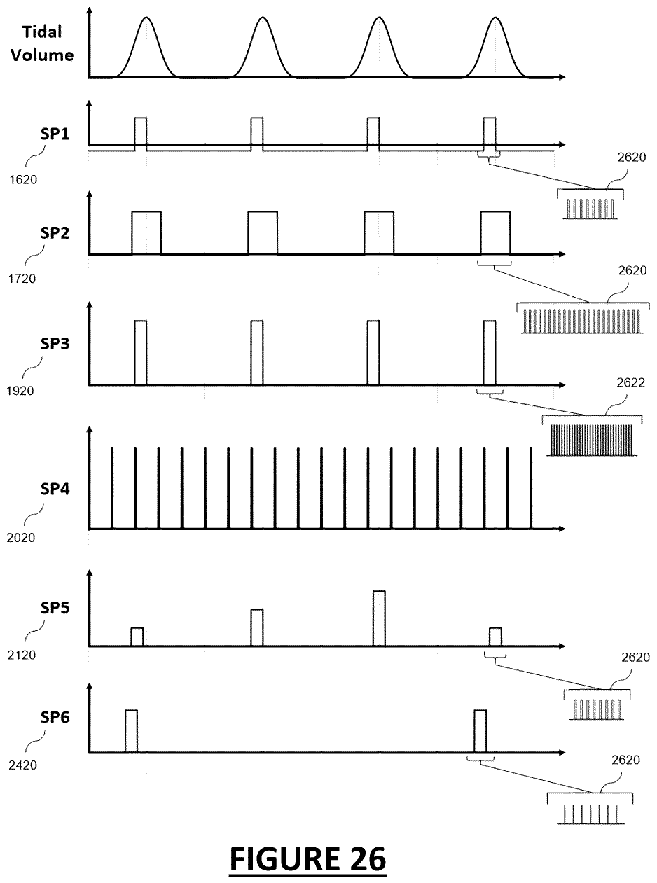

### FIG. 26: Stimulation Patterns 1-6

FIG. 26 depicts all previously discussed stimulation patterns (SP1-SP6) in relation to tidal volume. In FIG. 26, it can be seen how each stimulation pattern has set parameters of different amplitudes, durations, and frequencies to illicit different responses.

Stimulation Pattern 1 1620 has a low amplitude, ranging from 0.1 V to 1.5 V. Stimulation Patterns 2 1720 and 6 2420 have a medium amplitude, ranging from 1.5 V to 4.5 V. Stimulation Pattern 3 1920 has a high amplitude, between 4.5 V and 10 V. Stimulation Pattern 5 2120 can have a low, medium, or high amplitude, while Stimulation Pattern 4 2020 has a very high amplitude of 10 V to 15 V.

Stimulation Patterns 1 1620, 2 1720, 5 2120, and 6 2420 operate at a medium frequency 2620 of 10-30 Hz. Stimulation Pattern 3 1920 has a high frequency 2622 of 30-50 Hz, whereas Stimulation Pattern 4 2020 has a very low frequency, ranging from 0.1 to 1 Hz.

Stimulation Patterns 1 1620 and 3 1920 have a short duration of 100-500 ms. Stimulation Patterns 5 2120 and 6 2420 have a medium duration of 500 ms to 3 s, while Stimulation Pattern 2 1720 has a long duration of 3-5 s.

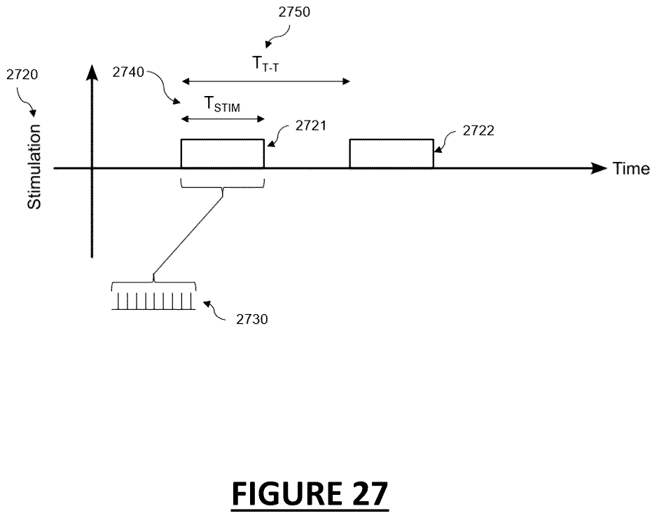

### FIG. 27: Stimulation Pattern 7

FIG. 27 illustrates stimulation pattern SP7 2720, designed as a main default operating mode as it does not require the availability of a reliable sensor signal. This pattern applies medium amplitude and frequency 2730 with a very long stimulation duration (T_{STIM}) 2740, ensuring continuous support without real-time feedback. SP7 2720 operates in an open-loop configuration, without synchronization to the respiratory cycle of the patient, and provides a consistent 1:1 stimulation ratio. The duty cycle could be any value, although it is preferred to be high, and it is usually set to a value equal or greater than 50%, meaning stimulation is active for the majority of each cycle to maintain efficacy without utilizing sensor signals. During periods of unreliable sensor signals, the algorithm (e.g., stimulation pattern 7) is to control delivery of stimulation asynchronously at a constant rate (e.g., a fixed rate)—such as 15 breaths per minute—and at a fixed duration. The fixed rate may be a predetermined stimulation rate that does not depend on the native respiratory rate of the patient to which stimulation is being applied. In this mode, stimulation is ideally delivered with a prolonged duration, such as 2 seconds or more, and may also be administered at a higher amplitude. Algorithm periodically checks the quality of the sensor signals, and when a reliable sensor signal is available, the algorithm automatically switches to synchronous operation, using sensor feedback to adjust the stimulation parameters in real-time by using one of the stimulation patterns of SP1, SP3, SP4, SP5 or SP6.

The following table shows example stimulation parameters that may be used in connection with certain example embodiments—including any or all of the stimulation patterns discussed herein. The listed ranges and values are examples and in certain instances may vary by, for example, about 5%, 10%, or 20% from the listed value/range for a given parameter.

Example Stimulation Parameters

| | Parameter | | | |
|---|---|---|---|---|
| | Amplitude | Amplitude | Frequency | Duration |
| | Unit | | | |
| | milli-Amperes (mA) | Volts (V) | Hertz (Hz) | Seconds |
| Very Low | | | <2 Hz | |
| Low | <1.5 mA | <1.5 V | 2 Hz-20 Hz | <0.1 |
| Medium | 1.5 mA-4.5 mA | 1.5 V-4.5 V | 20 Hz-50 Hz | 0.1-0.5 |
| High | 4.5 mA-10 mA | 4.5 V-10 V | >50 Hz | 0.5-2 |
| Very High | >10 mA | >10 V | | >2 |

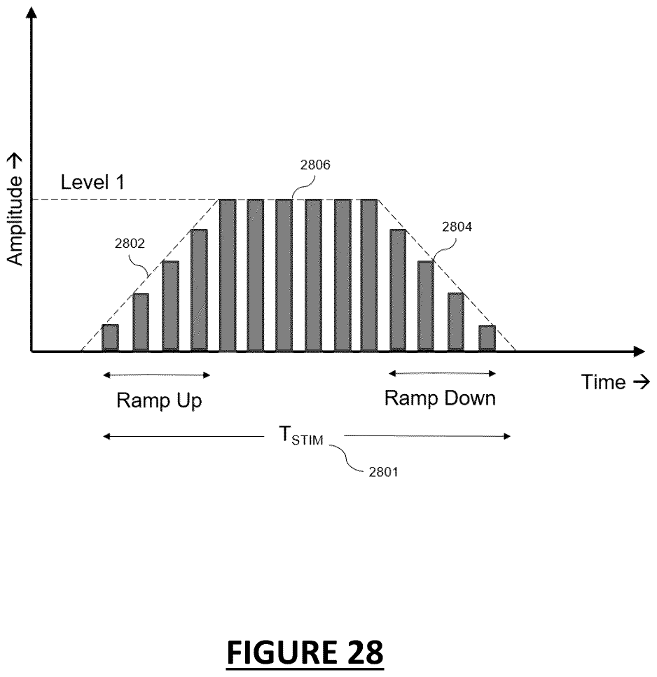

### FIGS. 28-31:

In order to cause a smooth contraction and the smooth relaxation of the target muscle, the stimulation that is applied to the nerve is ramped up 2802 and ramped down 2804 during the total stimulation duration 2801, as shown in FIG. 28. Briefly, the during the Ramp Up 2802 period, the stimulation amplitude is increased. In FIG. 28, the amplitude starts from zero and increases up to Level 1 2806. Although the ramp up 2802 that is shown in FIG. 28 has a linear slope, it is possible to use different envelopes defining the amplitude of the stimulation waveform during the ramp up phase. Similarly, the amplitude of the stimulation waveform is decreased from Level 1 2806 down to zero during the Ramp Down period, which could be linear or non-linear.

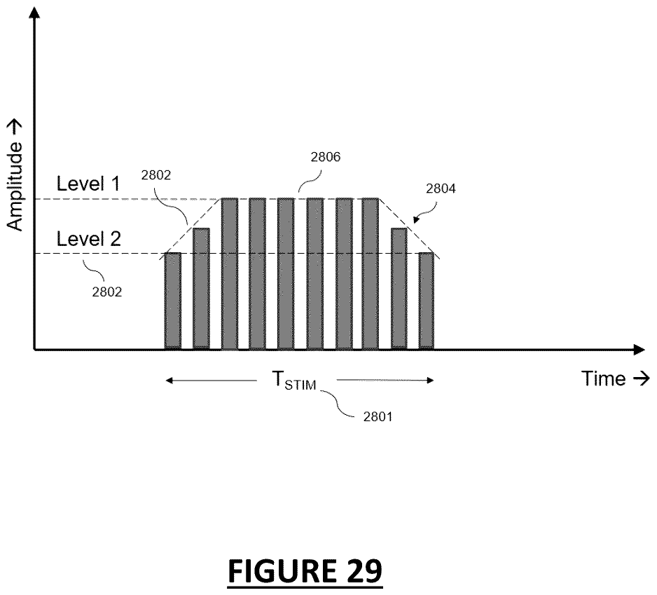

FIG. 29 shows the envelope of the stimulation waveform, again with linear Ramp Up 2802 and linear Ramp Down 2804 phases, however, this time the amplitude of the stimulation applied during the Ramp Up 2802 phase starts from Level 2 2902, instead of starting at zero or near zero. Similarly, the amplitude of the stimulation drops down to Level 2 2902 during the Ramp Down 2804 phase, instead of going all the way down to zero. Once again, the changes in the stimulation amplitude could be linear or non-linear during the Ramp Up 2802 and Ramp Down 2804 periods during the duration of stimulation 2801.

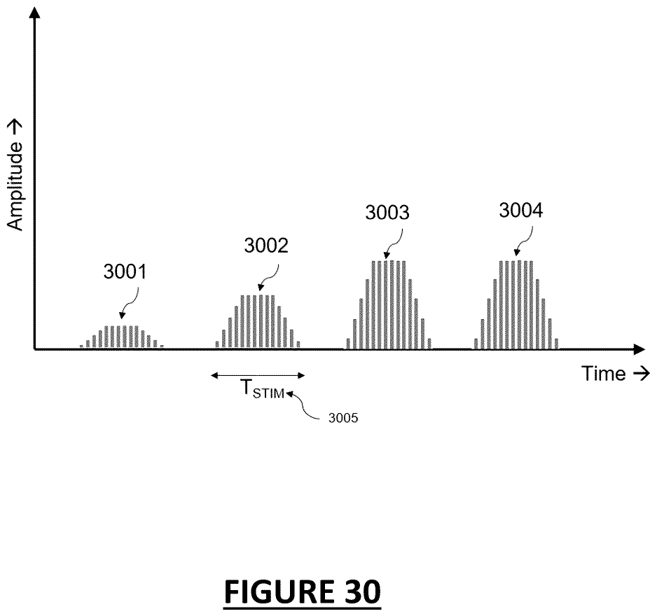

Treatment regime may be initiated upon a request from the patient, or based on fixed timing, or following an arousal or a posture change. In order to allow the patient to get accommodated to the stimulation, overall amplitude of the stimulation bursts are increased gradually, as shown in FIG. 30. First burst 3001 is at a low amplitude, the subsequent one 3002 is at a higher amplitude, and so on. Once the therapeutic stimulation level is reached 3003, the remaining stimuli 3004 are all applied at the same amplitude. Although FIG. 30 showed only two stimulation bursts before reaching a therapeutic dose, in general there could be more than two or less than two stimulation burst that may be needed before arriving at the therapeutic stimulation amplitude.

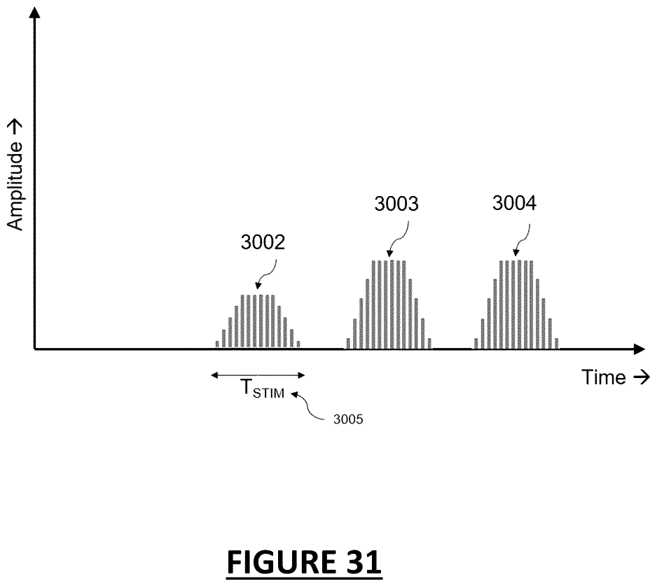

If the threshold of sensation is known, then it is possible to start the gradual increase of the amplitude of the stimulation bursts at or near the threshold of sensation by skipping the stimulation bursts with amplitudes that are below the threshold of sensation, as illustrated in FIG. 31. In this case, the stimulation burst 3001 is completely omitted as its amplitude is below the threshold of sensation.

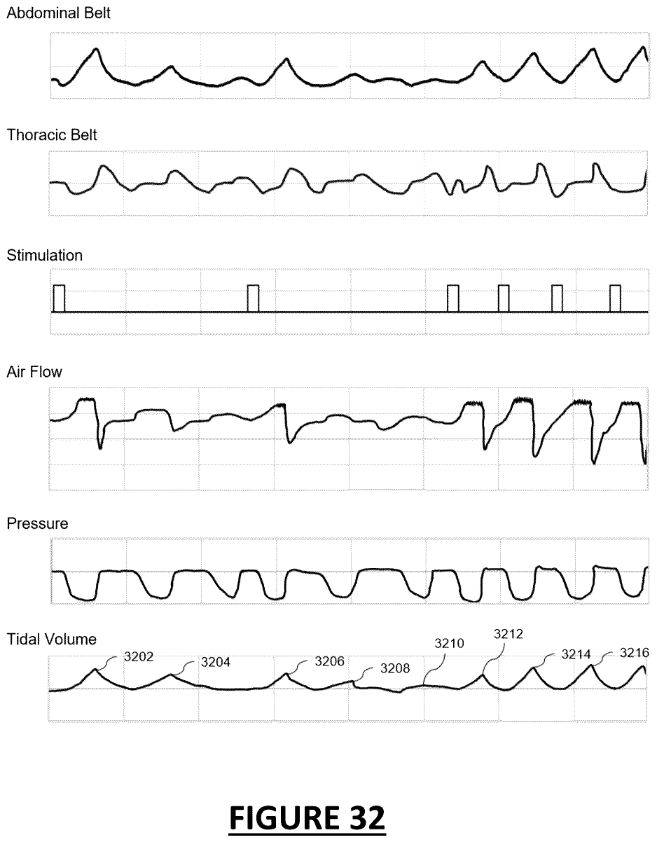

### FIG. 32: PNS at Endogenous Respiratory Effort

FIG. 32 illustrates clinical data demonstrating that phrenic nerve stimulation (PNS) delivered near the onset of the endogenous respiratory effort is more effective at restoring tidal volume, as seen by the first breath 3202, sixth breath 3212, seventh breath 3214, and eight breath 3216.

The effectiveness of PNS depends significantly on the phase of the respiratory cycle during which stimulation is applied, suggesting that the underlying physiological mechanisms are influenced by dynamic mechanical and neural factors. PNS administered during the late expiratory/early inspiratory phase—close to the onset of the endogenous inspiratory effort—is shown to be the most effective. Stimulation during this time frame typically results in greater airway opening and a steeper downslope of pressure responses. This enhanced response may be explained by the oscillating excitability of respiratory motor neurons across the respiratory cycle, with greater excitability during the inspiratory phase, making PNS-induced neural reflexes easier to activate and more effective. With stimulation that is delivered with the proper timing, good ventilation efficacies were achieved [3202, 3212, 3214 and 3216]. However, a poorly timed stimulation did produce a ventilation [3206] which was not significantly better than the ventilation obtained with no stimulation [3204, 3208 and 3210].

In contrast, PNS applied during the late inspiratory/early expiratory phase—toward the end of the breath—is less effective at producing airway opening. However, late-inspiratory PNS tends to prolong the inspiratory period and modestly improve ventilation by increasing the inspiratory duty cycle. This prolonged inspiratory effort may result in dynamic hyperinflation, which enhances caudal traction and contributes to improved upper airway patency.

### FIGS. 32-34 PNS Timing

FIG. 32 highlights timing in PNS delivery. In particular, FIG. 32 illustrates that stimulation near the onset of the endogenous respiratory effort can be optimal for achieving effective airway opening and tidal volume restoration.

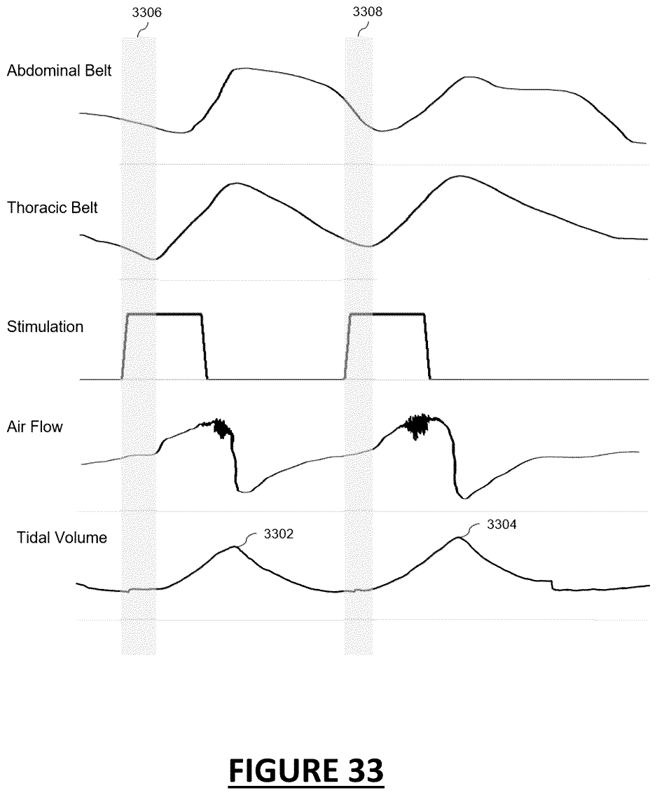

FIG. 33 illustrates the latency, or “lag,” observed between the onset of phrenic nerve stimulation (PNS) and the initiation of inspiratory airflow, signifying the relief of upper airway obstruction.

This latency provides valuable insights into the mechanisms driving airway opening. As shown in FIG. 33, there is a lag 3306 between the first onset of stimulation and the first onset of inhalation 3302, and a lag 3308 between the second onset of stimulation and the second onset of inhalation 3304. The duration suggests that the response is not a fast, monosynaptic reflex, such as those typically observed in somatosensory reflexes.

However, the observed first latency 3306 and second latency 3308 aligns with the possibility of a slower, polysynaptic reflex involving phrenic afferent projections to the brainstem. For instance, phrenic afferents may project to the nucleus of the solitary tract (NTS) in the medulla via ascending spinobulbar pathways. The NTS, which receives inputs from vagal afferents and carotid chemoreceptors, is a plausible site for such a reflex, though direct evidence of phrenic afferent inputs to the NTS is currently lacking.

During the latency period 3306 and 3308, the data shows that thoracic pressure becomes increasingly negative due to diaphragmatic contraction. This decrease in intrathoracic pressure is often accentuated when the diaphragm pulls against an obstructed or partially obstructed upper airway. It is likely that this negative pressure activates upper airway sensory afferents, which may contribute to airway opening and the resumption of inspiratory airflow, as depicted in FIG. 33.

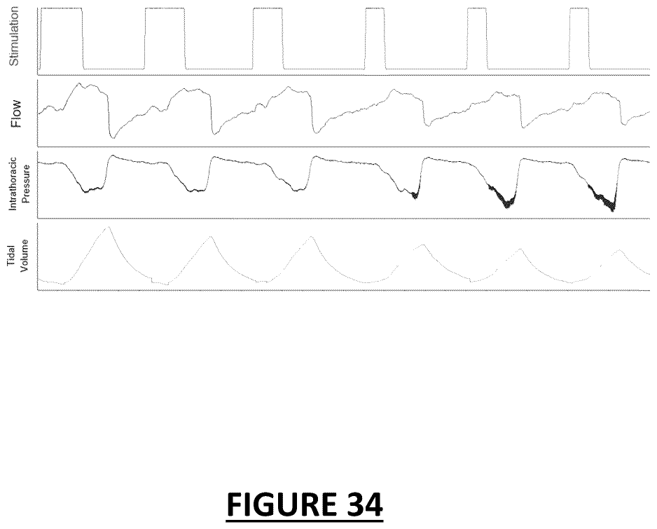

FIG. 34 shows the relationship between stimulation duration and air flow, intrathoracic pressure, and tidal volume. In this figure, as the stimulation duration decreases, the tidal volume, air flow, and intrathoracic pressure decrease as well. This data further emphasizes that the stimulation duration influences the ventilation efficacy, and usually the longer stimulation duration would result in better outcomes, which is why the stimulation pattern SP7 uses longer stimulation durations and higher duty cycles.

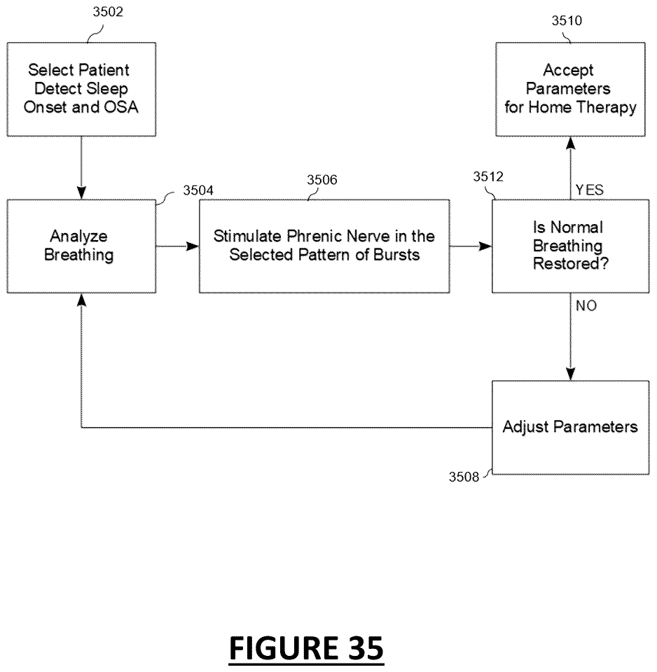

### FIG. 35: Therapy Selection Process

FIG. 35 illustrates a basic process of therapy selection that may be implemented in software/firmware that is used to control one or more aspects of therapy as discussed herein. Initially, a patient can be identified to have moderate or severe OSA based on standard home PSG test. For example, patient may have apnea hypopnea index (AHA)>20 events per hour. Patient is implanted with an IPG and a phrenic nerve stimulation electrode. IPG is confirmed operational, and patient is discharged for a period needed to heal, such as one month. Patient is brought to the office of the sleep physician specialist for therapy activation.

Once in the office, the process shown in FIG. 35 may be performed. While patient is sleeping in the office 3502 their breathing pattern and sleep pattern are analyzed by standard instrumentation used for sleep studies 3504. Stimulation of phrenic nerve is initiated at an initial set of parameters 3506. The parameters of possible stimulation patterns can be any of the discussed patterns, or patterns outlined in Table 4.

If the patient's OSA is resolved and AHI is reduced sufficiently, for example by 50%, patient may be sent home 3510 with a selected set of parameters and instructions to initiate therapy every night. If not, parameters can be changed 3508 and titrated upwards until OSA is resolved. For example, stimulation current (index of energy delivered to the nerve) can be increased. Increased current generally results in stronger diaphragmic contractions until muscle fibers are fused and the muscle cannot contract more. The rate at which amplitude of pulses in the bust is increased, often called ramp time, can also be shortened to generate (e.g., a more vigorous or abrupt) diaphragmic contractions.

Duty cycle, stimulation rate can also be increased with the understanding that some air trapping may occur during stimulation if stimulation bursts are more frequent or last longer. Some patients may benefit from lung volume increase during sleep to prevent lung collapse and loss of caudal traction exerted by the lung inflation on the airway. All stimulation parameters are titrated based on patient's tolerance. It is anticipated that after patient adapted to therapy, the intensity of stimulation may be increased.

In certain examples the timing of the stimulation can be synchronized to the phase of the respiratory cycle. In certain cases, such synchronization may be achieved in a clinical environment. However, in a home setting such synchronization may be more difficult to achieve when a patient has an implantable stimulator. To provide the implantable device with information regarding the phase of the respiration, various sensory systems, including implantable accelerometers and transthoracic impedance sensors, can be used in accordance with certain examples.

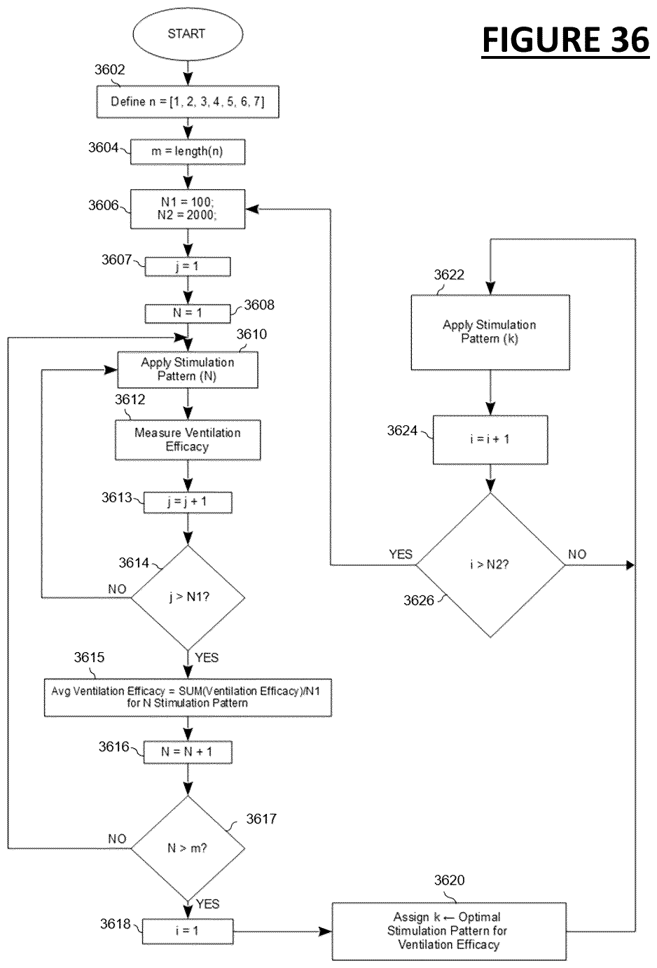

### FIG. 36: Stimulation Pattern Cycling

FIG. 36 illustrates a flowchart describing an algorithm for optimizing ventilation efficacy by cycling different stimulation patterns. The algorithm initializes by assigning all stimulation patterns to the vector n 3602, and length(n) is defined as m 3604, representing the total number of stimulation patterns available for cycling. N1 and N2 are initialized 3606, where N1 is a predetermined amount of cycles as defined by the system, physician, or patient to obtain enough data points for efficacy calculation, where N2 is the predetermined maximum number of applications before re-testing efficacy of a stimulation pattern. In this embodiment, N1 is set to 100 stimulations and N2 is set to 2000 stimulations. A counter j is initialized to 1 3607 to count through N1 cycles. N is also then set to 1 3608 to indicate that the first stimulation pattern, SP1 1620, is to be applied.

The first stimulation pattern, SP1 1620, is applied to the electrode to treat the patient 3610, while ventilation efficacy is recorded for N1 cycles 3612. With each ventilation efficacy stimulation applied 3610 and ventilation efficacy recorded 3612, j is incremented+1 3613 until j is greater than N1 3614. This helps to ensure that enough stimulations have occurred to obtain an accurate average of ventilation efficacy. For each cycle, the ventilation efficacy is recorded 3612. Upon completion, the ventilation efficacy data is averaged for that stimulation pattern over N1 cycles 3615. The counter N is then incremented by 1 3616 and checked against m 3617 to determine whether additional stimulation patterns remain to be tested. If N is not greater than m, the next stimulation pattern corresponding to N is applied 3610. This process continues incrementally through all stimulation patterns in the n-vector.

Once N is greater than m, indicating all stimulation patterns have been tested, the algorithm defines a new counter i, initialized to 1 3618. A variable k is then set to represent the optimal stimulation pattern 3620, determined as the pattern achieving the highest ventilation efficacy during the previous cycling. In this embodiment, the highest ventilation efficacy is determined by taking the average of the recorded ventilation efficacy over the number of cycles. In alternative embodiments of the algorithm, the highest ventilation efficacy may be determined using different statistical methods, such as the maximum value, median, or variance-based selection criteria.

The optimal stimulation pattern k is then applied to the electrode to treat the patient 3622, and the counter is incremented by 1 with each application 3624. If i exceeds N2 2626, the algorithm resets n to 1 3608 and re-applies all stimulation patterns in the n vector to validate efficacy under current conditions. If i does not exceed N2, the algorithm continues applying stimulation pattern k 3622 until the stimulation pattern has been applied N2 times. This process will continue for the duration of the patient's sleep, only pausing or terminating when data from sensor inputs confirm the patient is awake.

This process ensures that stimulation patterns are systematically tested, and the most effective pattern is identified and applied for optimal ventilation support, with periodic re-evaluation to adapt to changing patient conditions.

Below is an example pseudocode illustrating the implementation of the process for cycling through stimulation patterns to optimize ventilation efficacy.

TABLE 5
%Initialization
n = [1, 2, 3, 4, 5, 6, 7];

| | |
|---|---|
| m = length(n); | %Total number of stimulation patterns |
| N = 1 | %Counter for current stimulation pattern |
| N1 = 100 | %Maximum number of applications |
| | before cycling |
| N2 = 2000 | %Maximum number of applications |
| | before re-testing |

```
%Cycle Through Stimulation Patterns
Lable_1
  for N = 1:m
    for j = 1:N1
      ApplyStim(N);
      Measure VentilationEfficacy;
      VentilationEfficacy(N1) = currentVentilationEfficacy;
      end
      VentEff(N) = SUM(VentilationEfficacy)/N1;
    end
%Calculate Optimal Ventilation Efficacy
    k = max_element(VentEff); %where k is the stimulation pattern
giving the best ventilation efficacy
%Apply Optimal Stimulation Pattern
    for i = 1:N2
      ApplyStim(k);
    end
  end
Go to StimPatternCycling
```

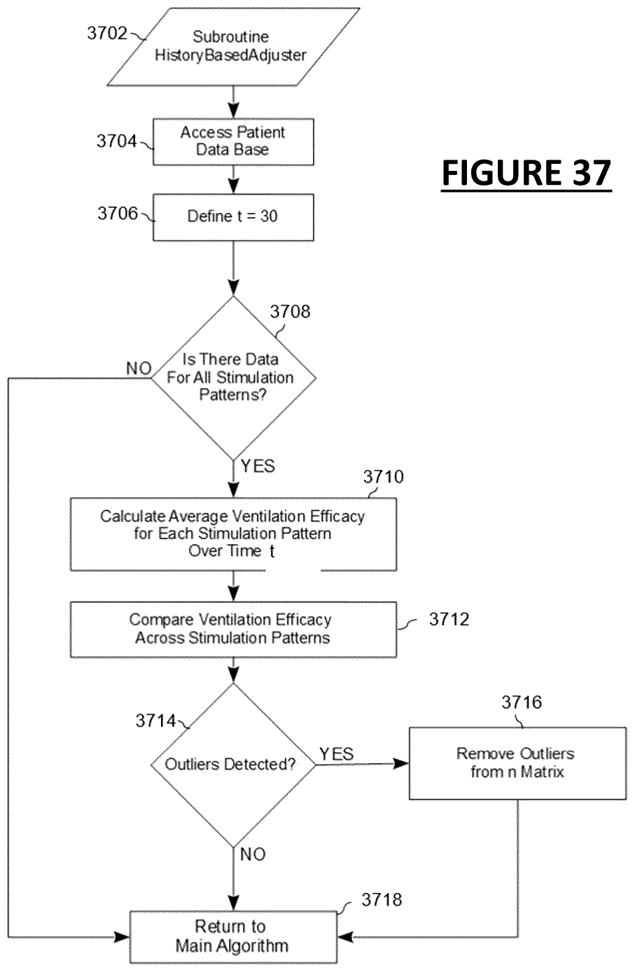

### FIG. 37: History Adjuster Process

FIG. 37 shows an embodiment of a flowchart illustrating the HistoryBasedAdjuster subroutine 3702, which refines stimulation patterns using historical patient data. During treatment, patient data is continuously stored in a database, such as an Electronic Health Record (EHR) system, a cloud-based platform, or a local server within a medical facility. This data is then accessed in real-time during the algorithm's runtime 3704. A time period, t, in days is defined to specify how far back the algorithm should retrieve data 3706. For instance, in this embodiment, if the patient has been undergoing nightly treatment for three months, the user or physician can set t=30, causing the algorithm to use only the most recent 30 days of data for adjustments.

Before administering treatment, the subroutine checks whether data exists for all stimulation patterns 3708. This step ensures that the algorithm does not incorrectly classify a stimulation pattern as an outlier simply due to missing data. For instance, if stimulation pattern 2 was deemed ineffective for the patient over the past month and thus not administered, and time t is set to one month, the system would lack the necessary data to determine if the pattern is truly ineffective. In such cases, the algorithm will revert to the main algorithm to reassess the ventilation efficacy of the stimulation pattern 3718. Alternatively, time t can be extended to include a period that contains relevant data.

If there is enough data for all stimulation patterns, the average ventilation efficacy is averaged for each stimulation pattern over time t 3710. The average ventilation efficacy can be calculated using various methods. For example, the arithmetic average is computed by summing the ventilation efficacy values for each pattern and dividing by the total number of data points within time t. Alternatively, the mean ventilation efficacy is determined by taking the average of the recorded values over time t. In some cases, a cumulative average may be used to track changes over time.

Once the average ventilation efficacy is calculated for each stimulation pattern, the algorithm compares the results across patterns 3712. This comparison can be made by evaluating the differences in average efficacy between each stimulation pattern. The algorithm may calculate the percentage difference in efficacy between patterns, identifying which pattern has the highest or lowest efficacy.

After comparing the ventilation efficacy across stimulation patterns, the algorithm identifies potential outliers in the dataset using various methods 3714. For example, outliers may be detected using the Z-score, where data points far from the mean are flagged, or the Interquartile Range (IQR) method, which identifies values outside 1.5 times the IQR from the quartiles. While the algorithm assesses the efficacy across stimulation patterns, it does not remove or penalize outliers in the top range, allowing high-efficacy patterns to remain in the analysis. The focus is instead on identifying low-range outliers that may indicate ineffective or underperforming stimulation patterns, which may need further adjustment or review. If outliers are detected, the algorithm removes the stimulation patterns associated with low ventilation efficacy from the n-vector 3716, preventing their application during treatment cycles. Alternatively, instead of detecting outliers, the algorithm may set a threshold value to flag patterns whose efficacy falls below a certain level and exclude them from the main algorithm.

Another alternative, detailed in the pseudocode of Table 6, is that the stimulation patterns are ranked based on efficacy, and a predefined number of the least effective patterns are removed from the bottom of the ranking. For example, if the predefined number is m=3, and the ranking of patterns from least efficacious to most efficacious is [2, 4, 3, 1, 5, 6, 7], the output n-vector would be n=[15,6,7], meaning only these patterns would be cycled in the main algorithm. By ranking the stimulation patterns according to their efficacy, the algorithm ensures that the most effective patterns are prioritized for future treatments, while less effective patterns are either reconsidered or removed from the treatment regimen. Any number of stimulation patterns can be removed from the cycling algorithm, that is, as long as that number is one less than the length of the n vector.

Below is an example pseudocode for the implementation of the HistoryBasedAdjuster subroutine.

```
TABLE 6
Subroutine HistoryBasedAdjuster:
%Initialization
  t = 30; %Where ‘t’ represents the number of days of past ventilation
efficacy data being utilized.
  m = 3;
%Check If All Stimulation Patterns Have Ventilation Efficacy Data
  if Stim1 == EMPTY or Stim2 = EMPTY or Stim3 == EMPTY or
Stim4 == EMPTY or Stim5 == Empty or Stim6 == Empty or
Stim7 == Empty:
    main_algorithm( ); %Loop to main algorithm detailed in
    Figure 36
  else:
    NP_VEffAvg = sum(Stim1)/length(Stim1);
    CT_VEffAvg = sum(Stim2)/length(Stim2);
    AS1_VEffAvg = sum(Stim3)/length(Stim3);
    AS2_VEffAvg = sum(Stim4)/length(Stim4);
    E_VEffAvg = sum(Stim5)/length(Stim5);
    NeuroP_VEffAvg = sum(Stim6)/length(Stim6);
    BM_VEffAvg = sum(Stim7)/length(Stim7);
    VentEFF_scores = [(“Stim1”, NP_VEffAvg), (“Stim2”,
CT_VEffAvg), (“Stim3”, AS1_VEffAvg), (“Stim4”,
AS2_VEffAvg), (“Stim5”, E_VEffAvg), (“Stim6”, NeuroP_VEffAvg),
(“Stim7”, BM_VEffAvg)];
    VentEFF_scores_sorted = sort(VentEFF_scores,
by_second_value_descending); %Sort
    filtered_VentEff_scores =
VentEFF_scores_sorted[0:length(VentEFF_scores_sorted) − m];
%Remove bottom 3
    filtered_VentEFF_scores =
filtered_scores(length(VentEFF_scores_sorted) − m, 1); Remove second
column
  % Create a mapping of names to numbers
    name_to_number = {
    “Stim1”: 1,
    “Stim2”: 2,
    “Stim3”: 3,
    “Stim4”: 4,
    “Stim5”: 5,
    “Stim6”: 6,
    “Stim7”: 7
    }
  % Loop through the vector to replace names with numbers
    for i = 0 to length(filtered_VentEFF_scores) − 1:
      % Extract the current name
      current_name = filtered_VentEFF_scores[i][0]
      % Check if the name exists in the mapping
      if current_name in name_to_number:
        % Get the corresponding number
        new_number = name_to_number[current_name]
      % Replace name with number
      n[i] = (new_number, filtered_VentEFF_scores[i][1])
      end if
    end
  end if
  main_algorithm( ); % Loop to main algorithm detailed in Figure 36
```

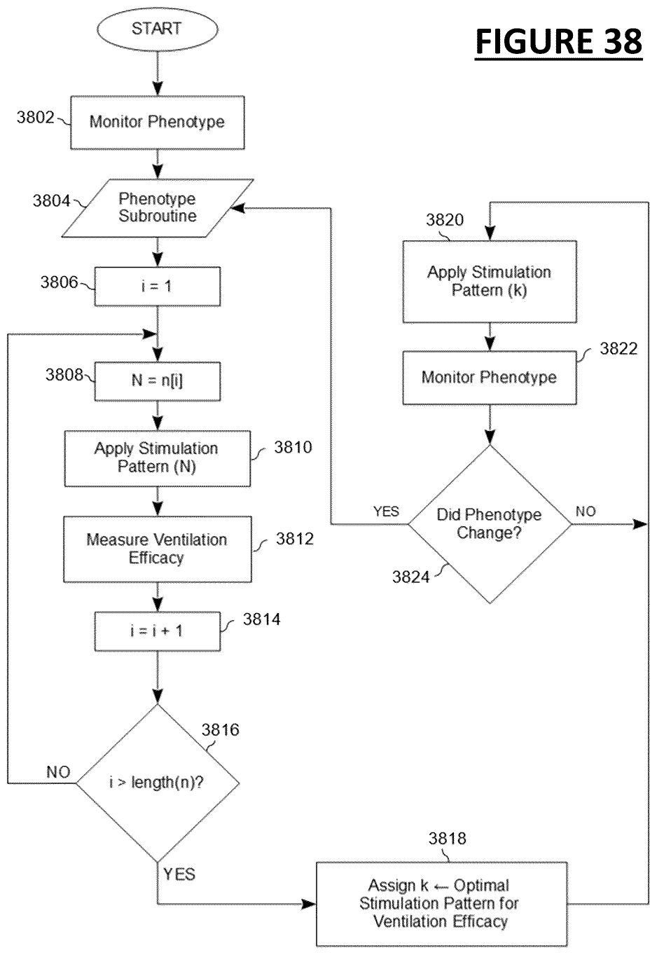

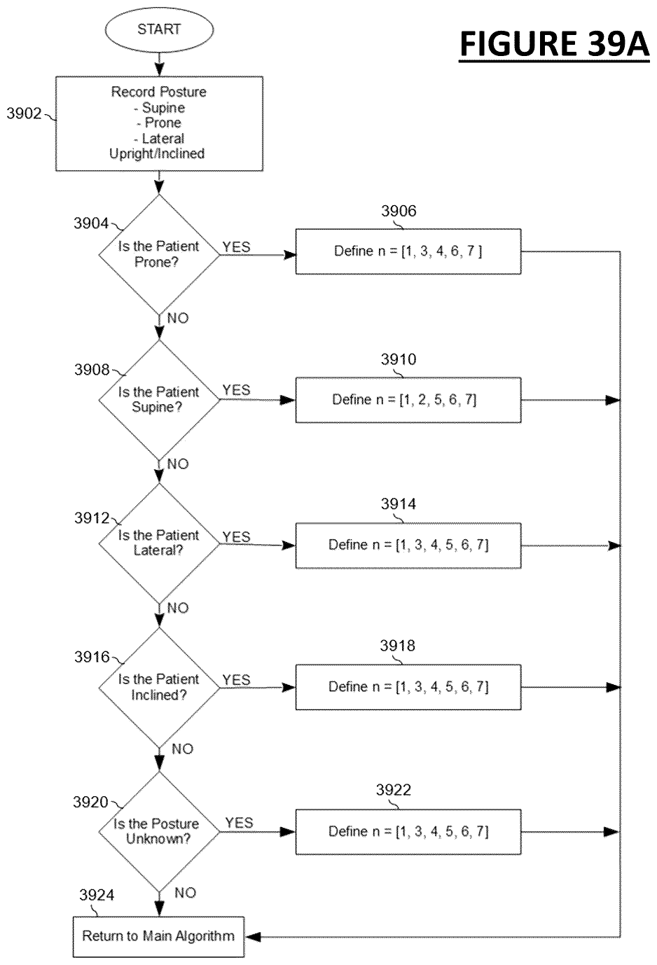

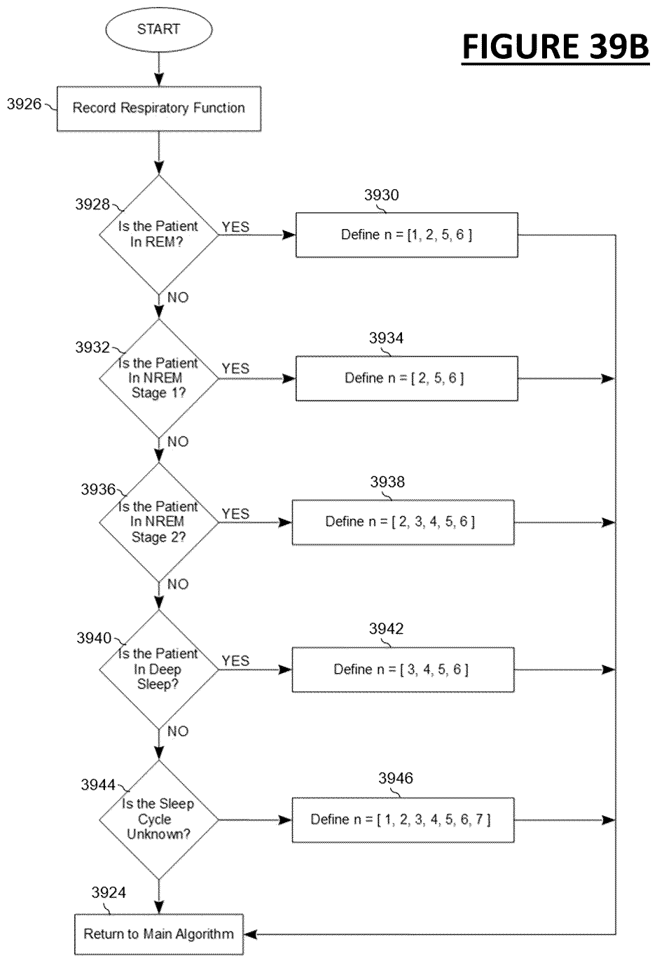

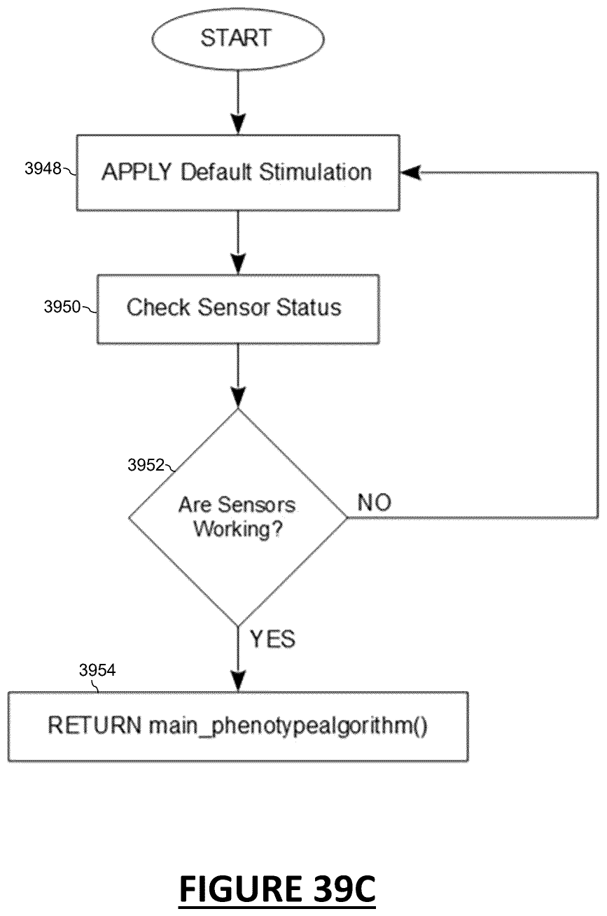

### FIG. 38-39C: Patient Phenotype Adjustment

FIG. 38 illustrates a different embodiment of the main algorithm, where the patient's phenotype dictates whether a stimulation pattern will be cycled during treatment. In this implementation, a phenotype—such as, but not limited to, sleep cycle, sleep position, substance use, or sleep deprivation—is monitored 3802. The specific phenotype being tracked can either be input by a physician based on the patient's condition or selected by an alternative algorithm that determines the optimal phenotype to monitor for treatment, based on past patient data. Once the phenotype is selected, a corresponding phenotype subroutine is executed 3804 based on the selected phenotype. For example, if posture is the phenotype being monitored, the algorithm will implement the subroutine described in FIG. 39A, which will be discussed shortly.

After the phenotype subroutine is executed 3804, the algorithm sets the iteration index i=1 3806, and the value of the first position in the n-vector is assigned to N 3808. The stimulation pattern corresponding to N is applied for a predetermined duration 3810, with the ventilation efficacy tested for each stimulation pattern over time t 3812. Once the average ventilation efficacy is calculated for each stimulation pattern, the algorithm compares the results across patterns using methods described prior.

The iteration index i is then incremented by one 3814. If i exceeds the length of the n-vector 3816, the stimulation pattern with the highest ventilation efficacy is assigned to the variable k 3818, and the corresponding stimulation pattern is applied 3820.

The phenotype is then continuously monitored for any changes 3822. If the phenotype remains unchanged, the stimulation pattern k continues to be applied 3820. However, if the phenotype changes, the phenotype subroutine is re-executed, and the algorithm loops, adjusting the stimulation pattern accordingly 2804. How the phenotype is monitored depends on the specific phenotype. For example, if the algorithm is basing stimulation patterns off of the patient's position, the phenotype would be monitored by changes in roll and pitch. That is, if a patient is in the supine position and a roll of greater than 80 degrees is recorded, the algorithm would adjust for the patient now being lateral.

Below is pseudocode for an embodiment of an algorithm shown in FIG. 38, where the options of phenotypes to monitor include position, sleep cycle, and sobriety. Phenotypes being monitored are not limited to this set.

```
TABLE 7
%%Functions
%Function to request and process input from the user
function request_input( ):
  % Display the options to the user
  print(“Please select an option:”)
  print(“1 - Posture”)
  print(“2 - Sleep Cycle”)
  print(“3 - Sobriety”)
  % Request input from the user
  Phenotype = input(“Enter your choice (1, 2, or 3): ”)
  % Process the user's choice
  switch choice do
    case “1”:
      print(“You selected Posture.”)
      subroutine_posture( )
      break
    case “2”:
      print(“You selected Sleep Cycle.”)
      subroutine_sleep_cycle( )
      break
    case “3”:
      print(“You selected Sobriety.”)
      subroutine_sobriety( )
      break
    default:
      print(“Invalid choice. Please select a valid option.”)
      request_input( ) % Recursively call to request input again
  end switch
end function
% Function to find the n value corresponding to the maximum VentEff
function optimal_stimulation_number(n, VentEff):
  max_index = 0% Initialize the index of the maximum value
  % Loop through VentEff to find the index of the maximum value
  for i = 1 to length(VentEff) − 1
    if VentEff[i] > VentEff[max_index] then
      max_index = i % Update the index of the maximum value
    end if
  end for
  % Return the corresponding n value
  return n[max_index]
end function
%%Algorithm
request_input( );
main(
%Cycle Through Stimulation Patterns
      for 1:SleepDuration
        run_phenotype_subroutine;
        for i = 1:length(n)
          for j = 1:N1
            ApplyStim(n[i]);
            Measure VentilationEfficacy;
            VentilationEfficacy(N1) = VentilationEfficacy;
            j = j+1;
          end
          VentEff(i) = SUM(VentilationEfficacy)/N1;
        end
%Calculate Optimal Ventilation Efficacy
        k = optimal_stimulation_number(n, VentEff)
%Apply Optimal Stimulation Pattern
        for i = 1:N2
          % Check if the phenotype has changed
            if phenotype_previous == phenotype_current then
              ApplyStim(k);
            else
        % If the phenotype has changed, rerun the algorithm
            main( )
            end if
        end
      end
)
```

The following subroutines allow prediction of the least efficacious stimulation patterns given a phenotype. The stimulation patterns removed from treatment given a phenotype parameter are removed by predicting which stimulation is least efficacious given anatomical and physiological reasoning.

FIG. 39A illustrates an embodiment of a subroutine designed to optimize the treatment of the main algorithm by basing treatment off of the patient's posture. In this subroutine, the n-vector is defined to contain all stimulation patterns. The patient's posture is then recorded using sensors such as accelerometers, gyroscopes, inertial measurement units (IMUs), or pressure sensors 3902. These sensors could be located in wearable devices (e.g., wristbands, chest straps), integrated into the mattress or bedding, or embedded within a pillow. The data from these sensors detects the position and orientation of the body, allowing the posture to be classified into one of four positions: supine, prone, lateral or inclined 3802. In other embodiments, there may be more or less positions, such as lateral being further defined as lateral left or lateral right. Once the patient's position is detected the algorithm removes the corresponding stimulation patterns from the n-vector. The stimulation patterns to not be used given a patient's position can be removed in any combination, based on the physician's, patient's, or system's input.

Once the patient's posture is recorded, the algorithm cycles through whether the patient is prone, supine, lateral, or inclined. FIG. 39A illustrates an embodiment of removing stimulation patterns based on their predicted physiological response. For example, if the patient is prone 2904, stimulation pattern 2—caudal traction and 6—neuroplasticity may be excluded from the n-matrix due to their limited effectiveness 2906. The prone position naturally promotes airway alignment by minimizing the collapse of soft tissues, a common cause of airway obstruction, particularly in obstructive sleep apnea (OSA). Gravity in this position helps maintain an open airway by positioning the head and neck in a way that counteracts airway collapse. Caudal traction, which applies mechanical force to stretch respiratory structures, offers little additional benefit in this posture since the airway alignment is already favorable. In contrast, methods like the negative pressure reflex are more effective, as they activate the body's natural mechanisms to maintain airway patency in response to any minor obstructions. Afferent stimulation targets the respiratory muscles, enhancing their function to support airway stability. Entrainment helps synchronize the patient's breathing pattern with external stimuli to regulate respiration. Neuroplasticity, although beneficial for long-term respiratory control, does not provide immediate relief from apnea events in the prone position, making it less effective in the short term. Therefore, the focus should be on methods that directly support airway function, with caudal traction and neuroplasticity excluded for this posture.

If the supine position is detected 3908, stimulation patterns 3 and 4—afferent stimulation can be removed from the treatment regimen as it is less effective than caudal traction and negative pressure reflex 3910. While afferent stimulation targets the respiratory muscles to improve their function, it primarily modulates muscle activity rather than addressing the mechanical factors that contribute to airway collapse. Caudal traction and negative pressure reflex, on the other hand, are more effective in the supine position. Caudal traction counteracts the gravitational forces that pull the tongue and soft tissues towards the back of the throat, helping to align the airway. Negative pressure reflex activates the body's natural mechanisms to maintain airway patency, responding quickly to minor airway collapses. Since afferent stimulation does not directly target the physical obstruction of the airway, it is less effective in providing immediate relief from sleep apnea events in the supine position, making it less critical for inclusion in the treatment algorithm.

If either the lateral left or lateral right sleeping positions are detected 3912, stimulation pattern 2—caudal traction can be excluded from the treatment regimen due to its minimal effectiveness 3914. The lateral positions naturally promote airway alignment and reduce the likelihood of airway collapse, as gravity helps prevent soft tissue obstruction. Since caudal traction applies mechanical force to stretch respiratory structures, it provides little additional benefit in these positions where the airway is already favorably aligned. These methods directly address airway collapse, making caudal traction less necessary and a less optimal choice for these postures.

If the upright or inclined position are detected 3916, stimulation pattern 2—caudal traction can be removed from the treatment regimen because it offers minimal additional benefit 3918. Gravity naturally helps keep the airway open by preventing the collapse of soft tissues, promoting a favorable airway alignment. Since caudal traction primarily works by applying mechanical force to stretch respiratory structures, its impact is diminished in the upright or inclined position where the airway is already aligned due to gravity. Negative pressure reflex and afferent stimulation are predicted to be more effective. Therefore, caudal traction is excluded from the treatment algorithm due to its reduced relevance in these postures.

If the algorithm does not detect that the patient is in any of the defined positions 3920, the subroutine will return to the main algorithm 3924 and cycle all stimulation patterns to test the most efficacious, as shown in FIG. 39A. However, other procedures may be implemented if no posture is recorded, such as the backup mode stimulation pattern being applied.

Below is an example pseudocode for the implementation of the Posture subroutine.

```
TABLE 8
%Function to determine patient's posture based on roll and pitch
function subroutine_posture(roll, pitch):
  % Evaluate the patient's posture using roll and pitch
    case roll >= −10 and roll <= 10 and pitch >= 70:
      return “Supine (lying on back)”;
        n = [1, 3, 4, 6, 7];
    case roll >= 70 and roll <= 110 or roll >= −110 and roll <= −70:
      return “Lateral (lying on side)”;
        n = [1, 2, 5, 6, 7];
    case roll >= −10 and roll <= 10 and pitch <= −70:
      return “Prone (lying on stomach)”;
        n = [1, 3, 4, 5, 6, 7];
    case pitch > 30 and pitch < 70:
      return “Inclined (partially upright)”;
        n = [1, 3, 4, 5, 6, 7];
    default:
      return “Unknown posture”
        n = [1, 2, 3, 4, 5, 6, 7];
end function
main_phenotype_algorithm( ); %Loop to main phenotype algorithm
detailed in Figure 38
```

FIG. 39B is an additional subroutine of the algorithm presented in FIG. 38, where the phenotype monitored is the sleep stage. Sleep stage can be monitored by one of or a combination of the following methods including respiratory monitoring using transthoracic impedance, heart rate variability analysis, eye movement monitoring, actigraphy, etc. For example, transthoracic impedance can be used to detect sleep cycle by monitoring impedance fluctuations. Typically, respiratory patterns vary across different sleep stages, allowing the algorithm to detect which sleep stage the patient is in. During REM, breathing is irregular and shallow, resulting in low and variable impedance amplitude with inconsistent frequency. In NREM Stage 1, breathing becomes more regular but may still show occasional irregularities, with moderate and stabilizing impedance amplitudes. NREM Stage 2 displays more regular and deeper breathing patterns, producing consistent and moderate-to-high impedance amplitudes with steady frequency. Deep Sleep, or NREM Stage 3, involves the slowest and most regular breathing, resulting in high and stable impedance amplitudes with low frequency. Methods to detect sleep stage may also be combined for more accurate results, such as monitoring heart rate variability.

In the embodiment illustrated in FIG. 39B, the sleep stages detected include REM, NREM Stage 1, NREM Stage 2, and Deep Sleep, and stimulation patterns are removed based on anatomical and physiological reasoning. The algorithm begins by recording respiratory function of the patient to classify what sleep stage the patient is in 3926. If the patient is in REM sleep 3928, the subroutine removes afferent stimulation (stimulation pattern 3 and 4) from the main cycling algorithm 2930. Caudal traction is less directly tied to autonomic control and is more useful in preventing airway collapse during lighter sleep stages. Negative Pressure reflex also primarily responds to airway obstruction which is more common in earlier sleep stages when muscle tone has not fully decreased.

If the patient is in NREM Stage 1 3932, the subroutine removes negative pressure reflex (stimulation pattern 1) and afferent stimulation (stimulation pattern 3 and 4) from the main cycling algorithm 3934. In this light stage of sleep, the body is still responsive to external stimuli and reflexive controls such as the negative pressure reflex, which helps keep the airway open. Since muscle tone hasn't fully relaxed, the negative pressure reflex is effective in responding to early signs of airway obstruction.

If the patient is in NREM Stage 2 3936, the subroutine removes negative pressure reflex (stimulation pattern 1) from the main cycling algorithm 3938. This is because in stage 2, when airway muscle tone is still decreasing but the airway is not fully collapsed, preserving negative pressure reflex is key to maintaining uninterrupted breathing. Phrenic nerve afferent stimulation serves as a backup, especially for those with weakened respiratory drive or mixed forms of sleep apnea, to ensure diaphragmatic activity is maintained and breathing does not falter.

If the patient is in Deep Sleep 3940, the subroutine removes negative pressure reflex (stimulation pattern 1) and caudal traction (stimulation pattern 2) from the main cycling algorithm 3942. In deep sleep, muscle tone is significantly reduced, therefore phrenic nerve activation can help maintain regular ventilation.

If the sleep stage is unknown 3944, the full set of n-matrix containing all stimulation patterns is used 3946. Once one of these five cases is detected the subroutine ends and returns to the main algorithm 3924 to cycle the stimulation patterns in the n-matrix.

Below is an example pseudocode for the subroutine sleep cycle to remove suboptimal stimulation patterns from the main algorithm testing.

```
TABLE 9
% Function to classify sleep stage based on transthoracic impedance
features
function classify_sleep_stage(impedance_signal):
  amplitude = calculate_amplitude(impedance_signal); % Average
impedance amplitude
  frequency = calculate_frequency(impedance_signal); % Respiratory
rate (frequency)
  variability = calculate_variability(impedance_signal); % Amplitude
  variability
end function
subroutine_sleep_cycle(amplitude, frequency, variability):
% Classify sleep stage based on extracted features
  if frequency > 20 and variability > threshold_high then
    return “REM Sleep” % Rapid and shallow breathing with high
    variability\
    n = [1, 2, 5, 6];
  else if frequency <= 20 and frequency > 15 and variability <=
  threshold_moderate
then
    return “NREM Stage 1” % Light sleep with moderate variability
    n = [2, 5, 6];
  else if frequency <= 15 and frequency > 10 and amplitude >
  moderate_threshold
then
    return “NREM Stage 2” % Light-to-moderate sleep with deeper
    breathing
    n = [2, 3, 4, 5, 6];
  else if frequency <= 10 and amplitude >= high_threshold and
variability <= threshold_low then
    return “Deep Sleep” % Slow, deep, and regular breathing
    n = [3, 4, 5, 6];
  else
    return “Unknown” % If criteria do not match known sleep stages
    n = [1, 2, 3, 4, 5, 6, 7];
  end if
end function
% Start the sleep stage detection system
main_phenotype_algorithm( ); %Loop to main phenotype algorithm
detailed in Figure 3
```

In the event that the sensors are not functioning, FIG. 39C illustrates an alternative embodiment of the main_phenotype algorithm. In this embodiment, the algorithm initiates by applying SP7 3948, main mode stimulation, as depicted in FIG. 39C, and attempts to verify the status of the sensors before collecting phenotype data 3950. If the sensors are operational, the algorithm proceeds with the standard process outlined in FIG. 36. If the sensors remain non-functional, the main mode stimulation pattern is maintained 3948, and the system periodically checks the sensors until reliable data can be obtained 3950. For example, the algorithm may verify sensor status after every five simulations or at one-minute intervals.

Sensor malfunctions can be identified by assessing signal noise levels, programming the system to recognize sensor behaviors indicative of an error, or simply verifying whether the sensors are outputting any data.

The algorithm depicted in FIG. 39C represents an embodiment that executes prior to the initiation of the main_phenotype algorithm. In another embodiment, it may also operate concurrently with the main algorithm. In this concurrent execution scenario, the algorithm in FIG. 39C continues to monitor sensor functionality even after the main_phenotype algorithm is running. If a sensor failure is detected at any point, the system immediately transitions to applying the default stimulation pattern to the patient.

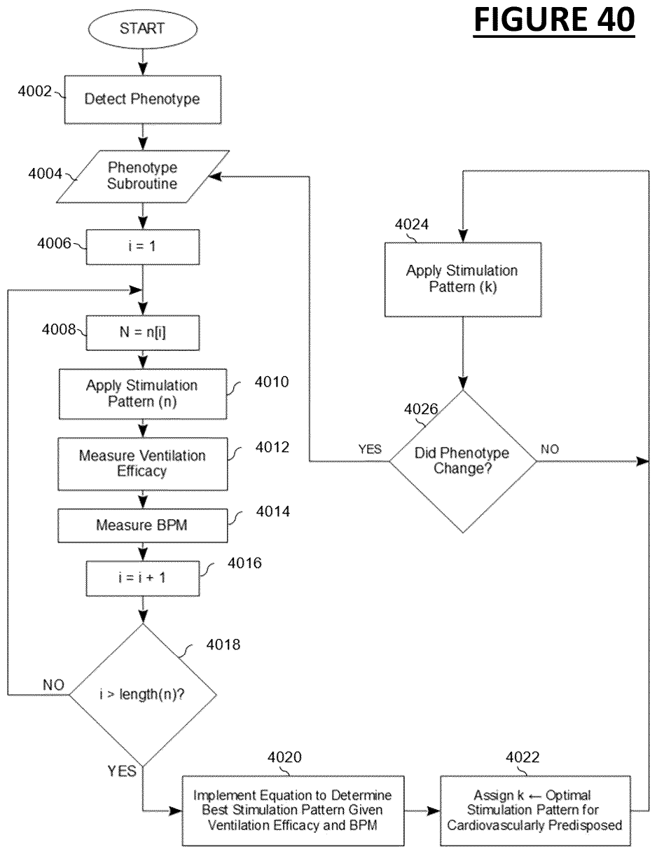

FIG. 40: Stimulation Pattern from Multiple Health Readings

FIG. 40 illustrates an embodiment of the phenotype algorithm that chooses the optimal stimulation pattern based on multiple health readings. In this embodiment, the optimal stimulation pattern is not chosen only based on ventilation efficacy, but also on BPM. Similar to previous embodiments, the algorithm begins by detecting a phenotype 4002 and removing stimulation patterns based on the indicated phenotype to reduce cycle time. The algorithm then cycles through and records patient response information during the cycling of the stimulation patterns. Patient response information now includes ventilation efficacy 4012 and beats per minute 4014. Once all stimulation patterns are cycled, the algorithm determines the optimal stimulation pattern based on BPM, ventilation efficacy, and/or AHI 4020. One embodiment for determining optimal stimulation patterns involving multiple variables is by minimizing their combined sum. For instance, consider a patient with an average BPM of 60 and an AHI of 15 for stimulation pattern 1, and an average BPM of 70 and an AHI of 10 for stimulation pattern 2. The algorithm evaluates the combined metric (e.g., average BPM+AHI) for each pattern and selects the pattern with the lowest value, ensuring the optimal balance across the variables.

In another embodiment, stimulation patterns may be excluded from cycling if any variable exceeds a predefined threshold during treatment. For example, the algorithm selects the stimulation pattern with the minimal AHI and continues its application only if the heart rate remains within an acceptable range, defined as not increasing beyond a specified limit per minute.

The embodiment of FIG. 40 enables doctors to account for multiple variables when treating patients, tailoring therapy to their individual needs. Cardiovascular diseases such as hypertension, coronary artery disease, and arrhythmias are closely linked to sleep disorders, and the effective treatment of OSA and CSA can significantly improve a patient's cardiovascular health. However, if a particular stimulation pattern induces irregular heart rhythms or elevated heart rates, it could potentially exacerbate existing cardiovascular issues. This embodiment is particularly beneficial for patients already predisposed to cardiovascular conditions, as it allows for the exclusion of stimulation patterns that negatively impact cardiovascular stability, thereby providing a safer and more effective treatment approach.

Once the optimal stimulation pattern is identified, the stimulation pattern is set to variable k 4022 and applied until the defined phenotype is changed 4026, and the algorithm continues until the patient awakens.

The pseudocode below illustrates an embodiment of determining the optimal stimulation pattern based on multiple variables, after different stimulation patterns have been applied and data has been collected.

```
TABLE 10
%AHI and BPM Data from Different Stimulation Patterns
%Negative Pressure Reflex
  NP_AHI = 10;
  NP_BPM = 60;
%Afferent Stimulation 1
  AS1_AHI = 15;
  AS1_BPM = 75;
%Afferent Stimulation 2
  AS2_AHI = 12;
  AS2_BPM = 70;
%Entrainment
  ET_AHI = 20;
  ET_BPM = 65;
%Calculating Optimal Stimulation Pattern
  NP_Stim = (NP_AHI + NP_BPM);
  AS1_Stim = (AS1_AHI + AS1_BPM);
  AS2_Stim = (AS2_AHI + AS2_BPM);
  ET_Stim = (ET_AHI + ET_BPM);
  Optimal_Stim = min([NP_Stim, AS1_Stim, AS2_Stim, ET_Stim]);
  Optimal_Stim = NP_Stim;
%Apply Optimal Stimulation Pattern
  k = 1; % Where 1 is the Negative Pressure Stimulation Pattern
  ApplyStim(k);
```

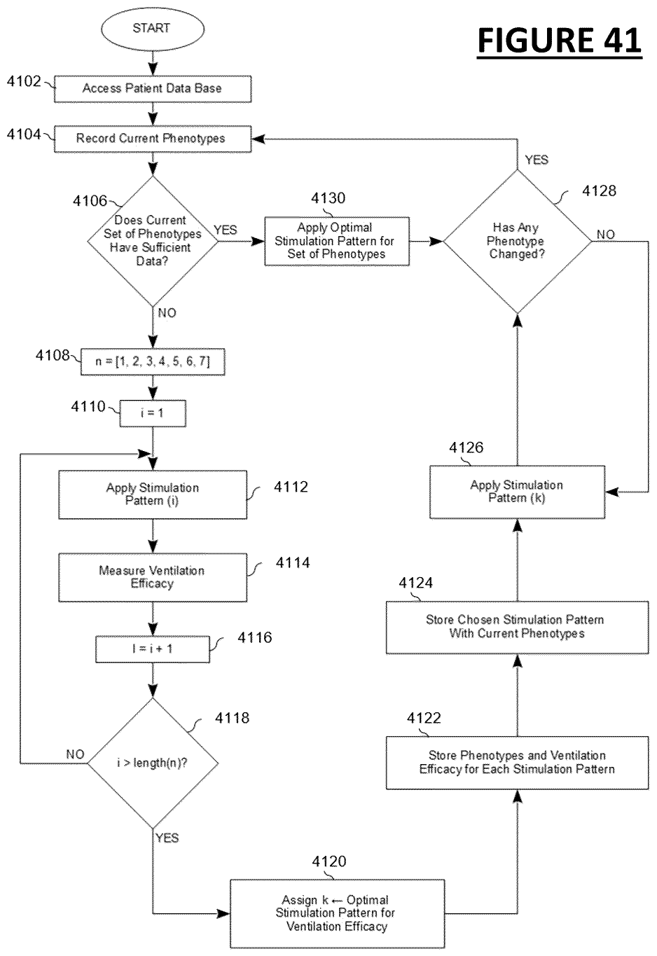

### FIG. 41: Stimulation Pattern Based on Historical Data

FIG. 41 illustrates an algorithm that selects and applies stimulation patterns based on historical treatment data gathered from various phenotype combinations. This algorithm records the patient's current phenotype among all measured phenotypes, and stores the optimal stimulation pattern used for that set of phenotype combinations.

The algorithm begins by accessing the patient database 4102 and recording the current defined phenotypes of the patient 4104. For example, the algorithm may be monitoring sleep position, sleep stage, and sobriety. In this case, the algorithm would record the patient's position, sleep stage, and sobriety, possibly resulting in the patient being prone, in REM sleep, and sober.

The algorithm then checks to see if there is any past data on this set of phenotype measurements 4106, that is, if the algorithm has cycled through and tested all stimulation patterns when the patient was prone, in REM sleep, and sober. If there is no data on this set of phenotype parameters, the algorithm cycles through the set of n stimulation patterns 4108, applying each one and measuring the ventilation efficacy for a predefined number of simulations 4114. Once all stimulation patterns have been cycled through 4118, the stimulation pattern with the highest ventilation efficacy is assigned to k 4120. The set of phenotypes is then stored 4122, along with the stimulation pattern and resulting ventilation efficacy 4124. The optimal stimulation pattern for the set of current phenotypes is also stored as data for when the patient is in the same position, sleep stage, and sobriety.

Once the data is stored, the optimal stimulation pattern k is applied 4126 until one of the defined phenotypes change 4128. For example, if the patient was previously prone, in REM sleep, and sober and moves on their side to be lateral, in REM sleep, and sober. Once a defined phenotype changes, the algorithm records the set of phenotypes, checks if there is sufficient data, and the algorithm loops.

If there is sufficient data for the set of phenotypes, the optimal stimulation pattern is applied 4130. This allows the patient to not have to undergo testing of stimulation patterns as often as previous algorithms, which can result in a more restful night sleep as the patient would have the maximum ventilation efficacy.

In another embodiment, if the phenotype is changing too quickly, applying the optimal stimulation pattern k can be done for a predefined amount of time or number or stimulations. After that predefined time, the algorithm checks if any phenotype has changed.

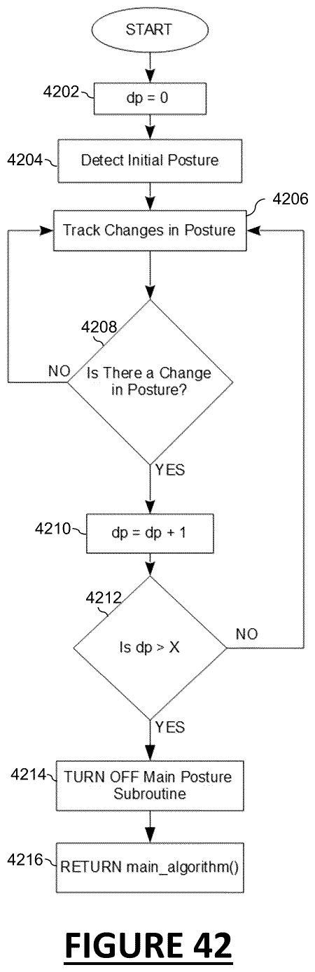

### FIG. 42: Posture Determination

FIG. 42 illustrates an algorithm designed to assess whether a patient's frequent posture changes make posture-based treatment ineffective. If a patient is changing posture too often, it is more difficult to collect data regarding the optimal stimulation pattern for different positions. Thus, treating sleep disorders based around patient posture may be ill advised. The algorithm begins by initializing dp=0 4202, where dp is the number of times a patient has changed posture. The algorithm then detects initial posture 4204, and continuously checks to see if the patient's posture has changed 4208. If the patient's posture has changed, dp is incremented by 1 4210 and checked to see if the value is greater than X, where X is the maximum threshold of posture changes 4214. That is, if dp>X the patient changes positions too often, and the main posture subroutine is ended, and the algorithm returns to the main_algorithm( ) 4216. In another embodiment, the main mode stimulation pattern could be applied if the patient is moving too much.

In another embodiment, calculating the threshold of maximum posture changes can be over a predefined time. For example, the prefined is set to 30 minutes, the algorithm calculates the number of posture changes over the past 30 minutes and evaluates whether the threshold has been met, where the threshold would not be the maximum allowed value of dp/time.

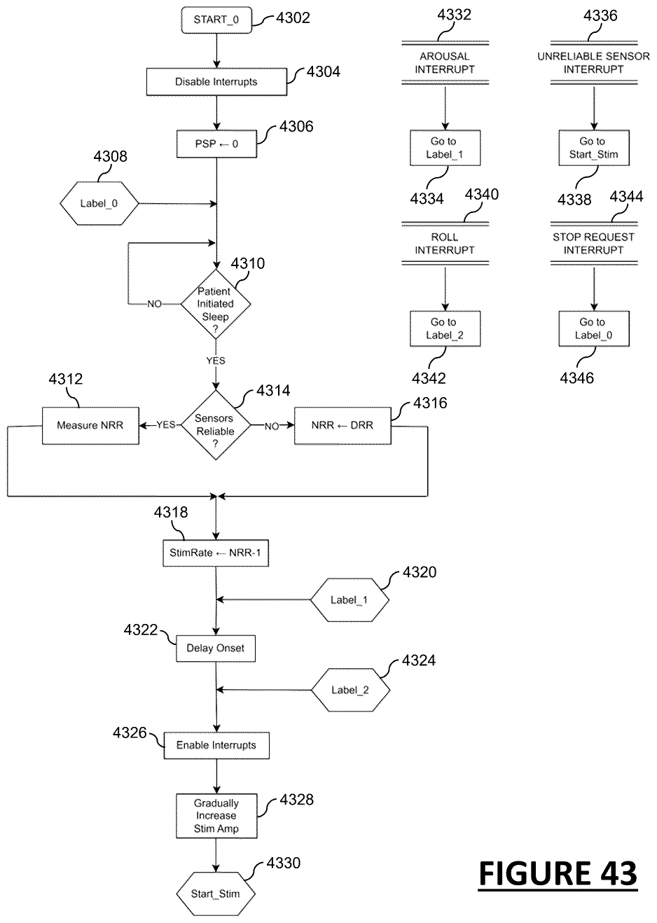

### FIG. 43-47: Additional Examples

FIGS. 43 through 47 show an alternate embodiment of an example algorithm that can be run on the implantable device. In this case, there are some interrupts that are used for:
Patient arousal,
Detection of unreliable sensor signals,
Patient rolls/change in posture, and
Patient request the stopping of the treatment.

As shown in FIG. 43, the algorithm initiates itself by first disabling all interrupts and setting the preferred stimulation pattern (PSP) into zero. Afterwards, the algorithm waits until the patient indicates that (s)he wants to sleep. At that instance, if the sensor signals are reliable, then the native breathing rate (NRR) is measured. If the sensor signals are not reliable, as preprogrammed default respiratory rate (DRR) is used as NRR. Stimulation rate is chosen slightly below the NRR, such as NRR—1 breaths per minute (bpm). A delay period is given before the interrupts are enabled for full function operation. Afterwards, the stimulation amplitude begins to increase gradually, as shown in FIGS. 30 and 31. Eventually the algorithm reaches to the Start_Stim, which is when the therapy begins in earnest.

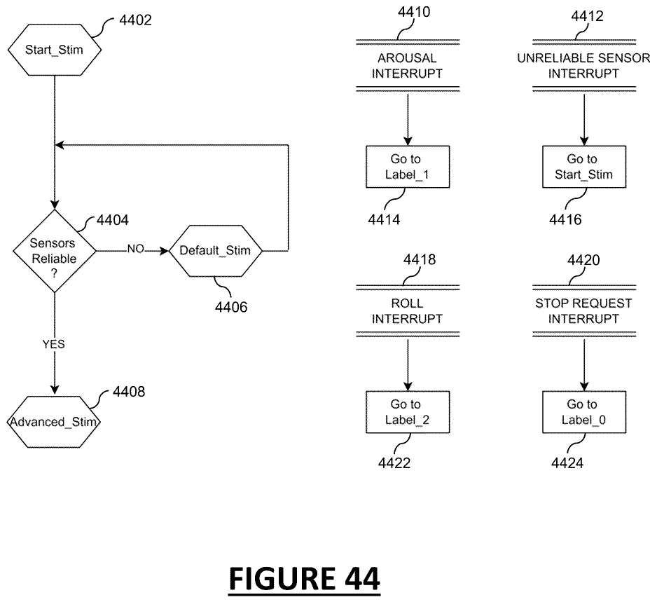

FIG. 44 shows that continuing from the Start_Stim position in the algorithm, reliability of the sensor signals is confirmed. If the sensor signals are deemed unreliable, then the default stimulation pattern of SP7 is used. If the sensor signals are reliable, then the algorithm moves to the Advanced_Stim portion. It should also be noted that at any time the sensor signals become unreliable, the interrupt subroutine bring the algorithm to the Start_Stim position, which will assure returning back to the Default stimulation pattern of SP7.

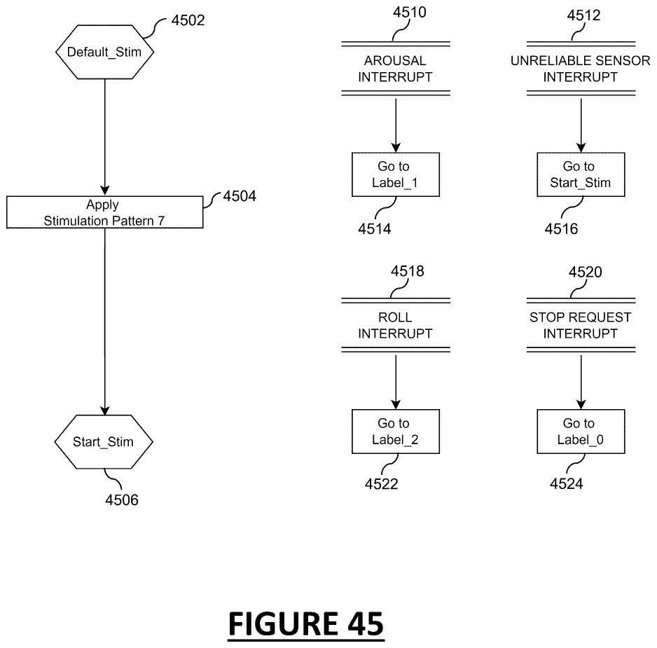

FIG. 45 shows the simple execution of the default stimulation portion of the algorithm where the stimulation pattern SP7 is applied and the algorithm returns back to Start_Stim to see if the sensor signals are reliable or not, as mentioned above.

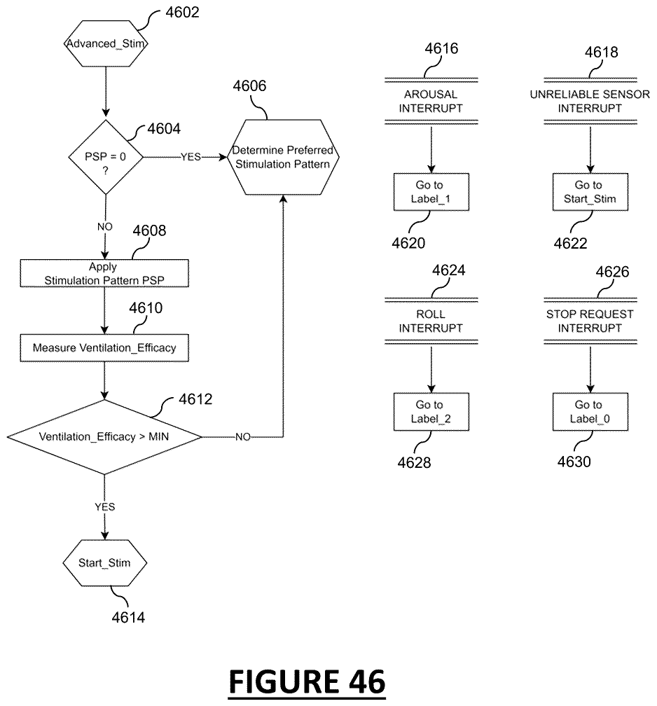

FIG. 46 shows the execution of the Advanced_Stim portion of the algorithm, If the preferred stimulation pattern (PSP) is known, then it is applied to the patient and the resulting ventilation efficacy is measured. If the PSP is not known or the resulting ventilation efficacy is poor, then a search is initiated for the determination of the preferred stimulation pattern.

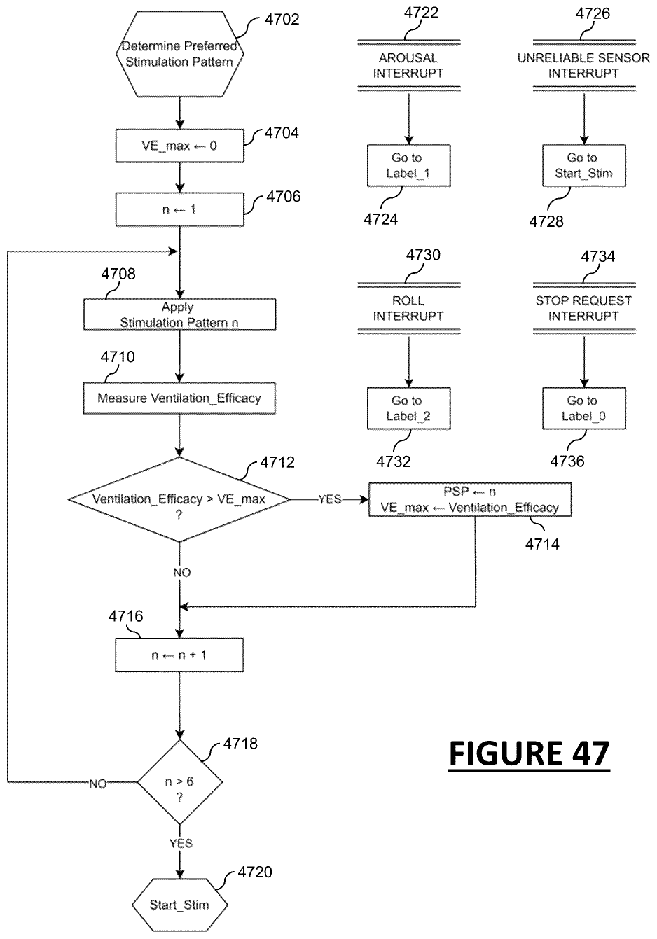

Finally, FIG. 47 shows the procedure followed for the determination of the preferred stimulation pattern. Briefly, the stimulation patterns SP1, SP2, SP3, SP4, SP5 and SP6 are successively applied, and the resulting ventilation efficacy values are measured. The simulation pattern that results in the maximum ventilation efficacy is chosen as the preferred stimulation pattern, PSP.

As mentioned before, during the execution of the above-mentioned segments of the algorithm, the interrupts remain enabled, so that upon the detection of arousal of the patient, loss of sensor reliability, patient roll or request for stop, the algorithm takes the proper action, as shown with the “Go to” steps on FIGS. 43, 44, 45, 46, and 47.

In some examples, the techniques discussed herein can be applied towards the treatment of central sleep apnea (CSA) and/or mixed sleep apnea (MSA). During CSA, subjects can have a very limited or no effort for breathing. MSA, which is sometimes referred to as Complex Sleep Apnea Syndrome (CompSAS), is a condition with components of both the central and the obstructive apneas, where the breathing stops due to both lack of effort and/or physical airway blockage. MSA may start as a central sleep apnea with no effort to breath, but may eventually transition into obstructive sleep apnea with a blocked airway during a single episode.

In some examples, treatment of CSA and/or MSA can be performed using various techniques including the techniques below:

### Continuous Stimulation for Central and Mixed Apnea (CSCMA):

In certain examples, stimulation is delivered to the patient at a predetermined respiratory rate, and sensory feedback is used for (e.g., to ensure) that stimulation effectively captures the phrenic nerve and causes the contraction of the diaphragm. This may include one or more of the following features for CSCMA:

Stimulation rate of CSCMA may be a rate that is, for example, predetermined while the subject falls asleep. During the sleep, the rate may then be adjusted. For example, if the sensors, such as the transthoracic impedance (TTI) or accelerometer (ACCEL), detect that the patient is breathing on his/her own (native rate) at a different rate than the predetermined rate, then the breathing rate of the patient is set based on (e.g., to) that native rate.

In some examples, native rate determination for CSCMA may be performed. In some examples, native rate for the CSCMA can be determined using the available sensors that are in the implanted device or stationed externally and communicate with the implanted device via telemetry. Sensors can be of different types (e.g., accelerometer, transthoracic impedance, inductance or impedance plethysmography, microphone, milli-meter wave radar, etc.).

In some examples, respiratory events can be detected from time and/or frequency domain analysis of the sensor signals. In time domain analysis, the peak detection techniques are used for the identification of the respiratory events. If detected respiratory events do not line up with stimulation timing, then the native respiratory rate is estimated from the detected event. As noted herein this will eventually be used as the stimulation rate of CSCMA. For frequency domain processing of the sensor signals, the rate (frequency) of the detected events can be compared with the rate of the stimulation that is detected. This can be accomplished with frequency domain techniques, such as a Fourier transform. If the two rates differ, then the detected rate is adopted as the new native rate.

In some examples, the parameters of CSCMA can be adjusted. For example, parameters of the stimulation delivered to the patient can be adjusted so that the stimulation captures the phrenic nerve and its efferent fibers effectively. This can result in contraction of the diaphragm. The parameters to be adjusted can include: 1) Amplitude: constant current, constant voltage, 2) Pulse width, 3) Frequency, and/or 4) Duration.

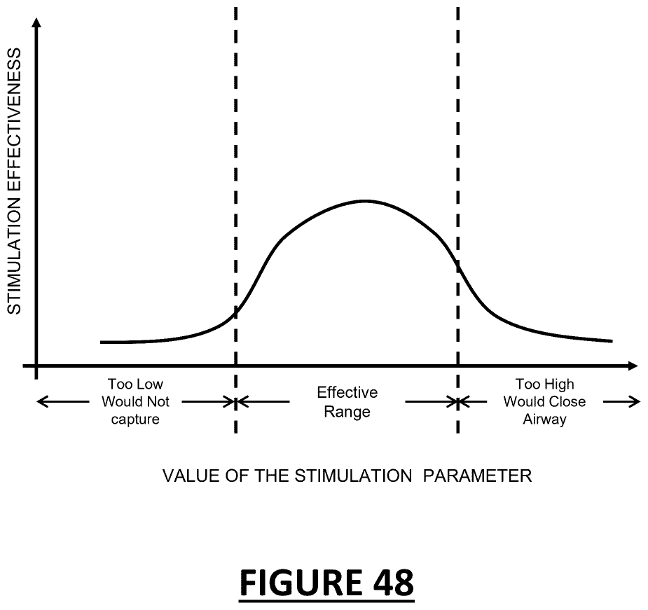

In some example, there may be an effective range for these (or other) stimulation parameters. When a stimulation parameter, such as the amplitude, is below its minimum value, then stimulation provided can to capture the phrenic nerve, and thus not cause a diaphragmatic movement. Similarly, when the value of the parameter exceeds its maximum, then the excitation provided to the diaphragm and the resulting diaphragmatic movement may be too strong and cause a collapse of the upper airway. This may then prevent the air intake to the lungs by the patient. Accordingly, it can be desirable to set the parameters in their effective range. This is illustrated in FIG. 48. The settings of these parameters may be important for the treatment of mixed sleep apnea, since the patient has already demonstrated a propensity for obstructive events. If the stimulation parameters were to exceed their maximum values, this may cause over stimulation and further exacerbation of the obstructive event (including completely closing the airway).

Accordingly, in certain example embodiments, one or more algorithms may be used in order to keep the stimulation parameters in their effective range A first algorithm can include the following steps:
Step 1: Set default values;
Step 2: Deliver stimulation N times, while recording sensor signals;
Step 3: If there is not a detected breath following each stimulation, then increase the value of the stimulation parameter;
Step 4: If there is a detected breath following each stimulation and the patient is experiencing an arousal, then decrease the value of the stimulation parameter; and
Step 5: Goto Step 2.

In the above-described algorithm, the arousals are used as an indication of ineffective stimulation, indicating that the stimulation parameters were set beyond their maximum effective range. Furthermore, the above-described algorithm can be used for the adjustment of stimulation parameters one-at-a-time, or for the adjustment of multiple stimulation parameters at the same time. In certain example embodiments, the value of N used in the algorithm can be chosen at will. In some examples, the value of N may be (e.g., required to be) more than one. In some examples, the value of N may be five or higher.

### Demand Stimulation for Central and Mixed Apnea (DSCMA):

In this mode of operation, an algorithm may be provided to provide stimulation such that it is delivered based on detection apneic events. Accordingly, stimulation maybe provided only when apneic events are detected

Operation of the DSCMA is similar to the CSCMA algorithm, with the following two main differences. First, the DSCMA algorithm may include functionality for withholding stimulation until an arousal or an apnea is detected. Second, in some examples, the DSCMA algorithm may deliver therapy for a predefined period of time, such as 15 minutes, and then suspend it again until the detection of another arousal or an apneic event.

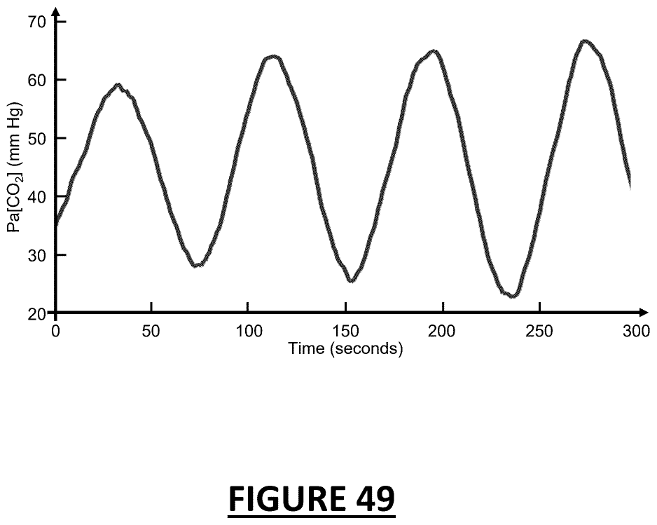

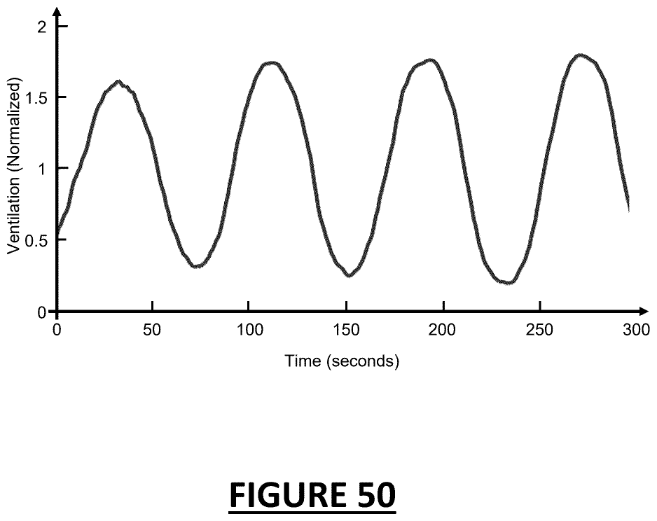

As mentioned earlier, the techniques discussed herein may provide methods of treatment for the mixed apneas as well. Mixed apneas generally start as central apneas where the patient makes no effort to breathe. If left untreated, such apneas with central origins lead into obstructive events, as the hypoxic conditions developed during the prolonged central apneas create excessive ventilatory drive, which in turn generate strong efforts to inhale in the absence of upper airway opening, eventually leading to an arousal, and repeat of the ventilatory cycle, as illustrated in FIGS. 49 and 50.

The oscillatory cycle shown in FIGS. 49 and 50 can be broken by using stimulation delivered to the patient—in accordance with the example embodiments discussed herein. In some examples, stimulation requires the IPG to estimate the oxygenation status of the patient from his/her respiratory activity, and then to deliver (or cause to be delivered) the stimulation ahead of time to prevent the patient from entering into the ventilatory cycle of arousals and apneas. The following algorithms can be used for determination of the timing for stimulation. These algorithms are discussed in the context of a Model Predictive Controller and a Short Demand Controller. These are discussed below.

It will be appreciated that the description of the Model Predictive Controller and the Short Demand Controller includes references to stimulation and its timing. This are made for the stimulation to be delivered to a breath. In other words, when the stimulation is on, the corresponding breath is stimulated, and when the stimulation is off, the breath is not stimulated. Accordingly, in some examples, the time of stimulation refers to whether breath is stimulated, not when within the breath the stimulation is delivered.

### Model Predictive Controller:

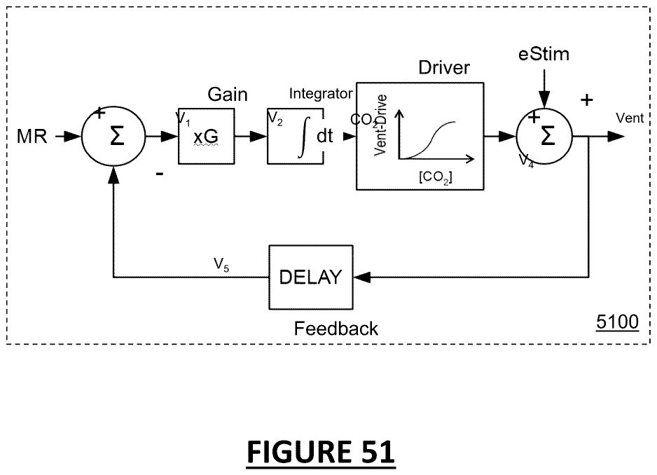

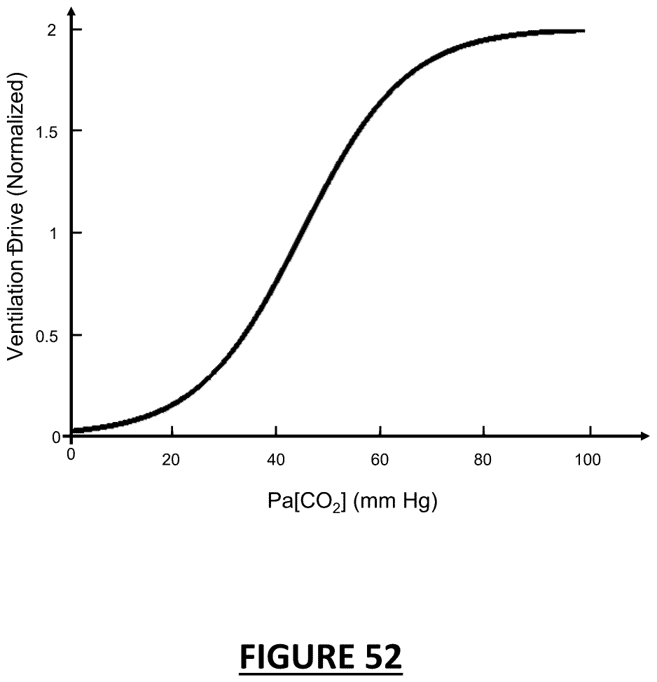

A model 5100 of the respiratory system that can be used for the treatment of the central and mixed apneas is shown in FIG. 51, while the ventilatory drive is shown in FIG. 52. In some examples, the goal of the stimulator may be to keep the Pa[CO₂] around 45 mm Hg (which may be the linear operation around the nominal respiratory range where the respiratory drive saturates at high and low values of Pa[CO₂].)

To determine when to deliver stimulation, a model predictive controller can be used, as describe below:

A real time dynamic optimizer is used, where at step n, the goal of the optimizer is to:

$$minimize {\sum }_{i = n}^{n + m} {[ {( PredictedOutput )}_{i} - {( DesiredOutput )}_{i} ]}^{2}$$

where the PredictedOutput for the future is estimated using the model shown in FIG. 51 and the DesiredOutput is the value of the Pa[CO₂] which is to be kept 45 mm Hg, or the corresponding value of the respiration with a nominal value of 1.0, and m is the observation horizon which is the length of the future the controller evaluates.

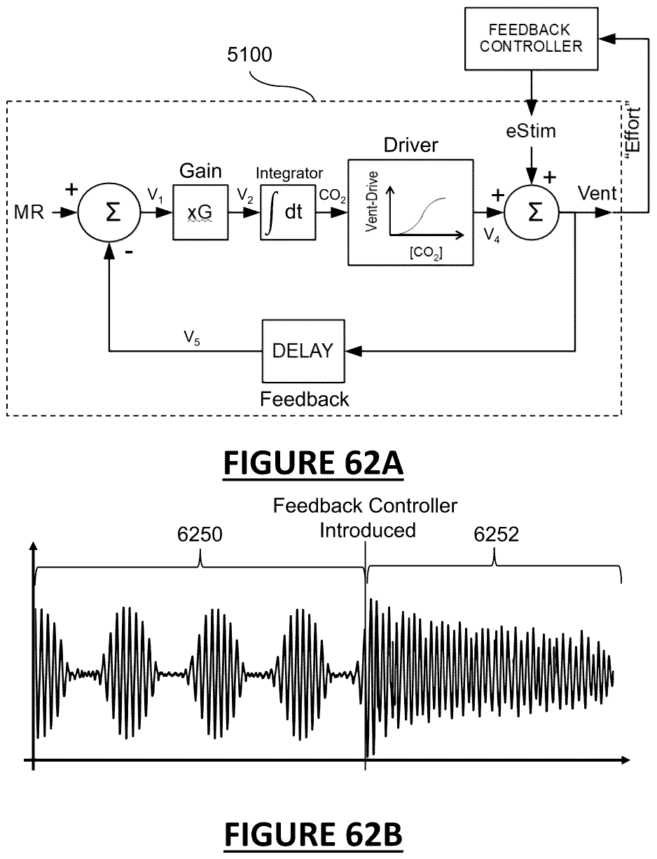

Operation of the model predictive controller is further illustrated at 6200 (which may be the same or similar to model 5100 from FIG. 51) in FIG. 62A. Briefly, the model predictive controller uses an approximate linear model of the system, where: 1) Internal feedback is allowed, 2) Internal delays, even in the feedback path, are allowed, 3) only the available inputs, which is the stimulation to be delivered, are modulated, 4) evaluations of the controller response is done only for the observation horizon, which is usually choses as [Observation Horizon]=2×[Circulatory Delay], 5) stimulation is done for only the first input, and 6) the controller is implemented as a Bang-Bang Controller (e.g., Stimulate (1) or Not Stimulate (0)).

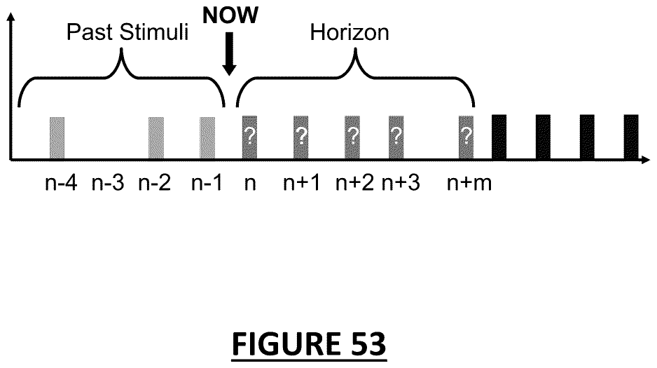

As shown in FIG. 53, at each step n, the model predictive controller runs an m-step simulation using the model shown FIG. 62A with the goal to according to the above equation.

Selection of the future inputs, or the stimuli to be applied, can be done using different methods. These include a heuristic search algorithm, an exhaustive search algorithm, or a look up table. For the examples described below, an exhaustive search algorithm was used for the determination of the optimal stimulation pattern, e.g., whether or not to deliver stimulation at a given point in the future which is within the Observation Horizon.

The following is an example simulation that demonstrates the performance of the model predictive controller.

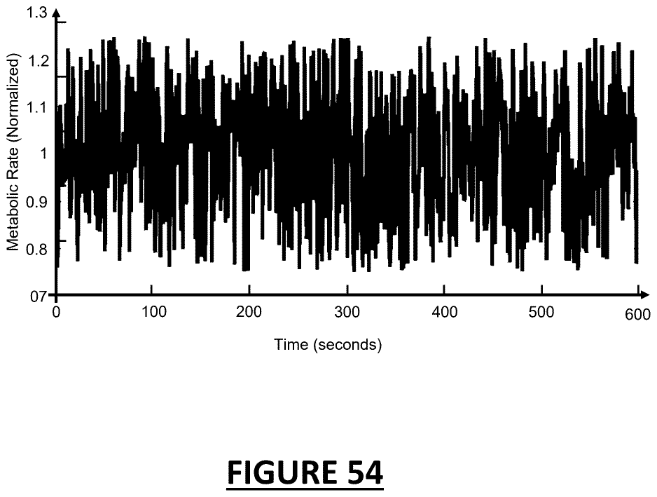

FIG. 54 shows the simulated metabolic rate of the patient. In general, the metabolic rate may not change significantly during sleep. Accordingly, in certain examples, for the Model Predictive Controller, a time varying metabolic rate with 25% variability (or other as-needed value) can be used. This is illustrated in FIG. 54 that shows a simulated metabolic rate with the random 25% variability.

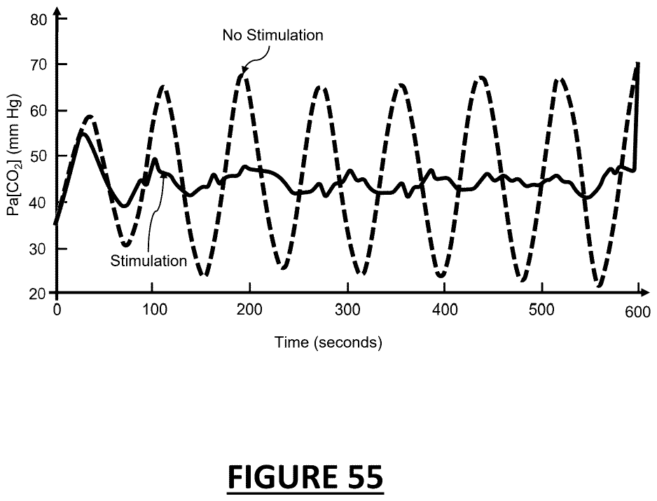

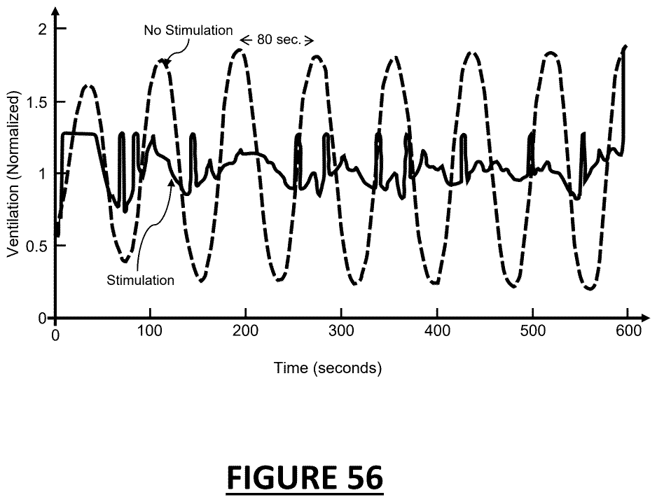

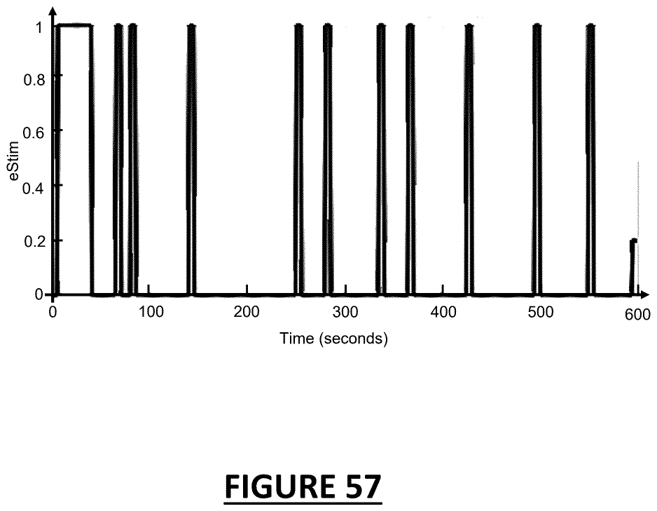

FIG. 55 shows the time course of Pa[CO₂] for a patient with a high respiratory loop gain value of 2.0. It should be noted that in healthy individuals, the respiratory loop gain value may be less than 1.0. Simulations were started at a perturbed value of Pa[CO₂], such as 37 mm Hg for example. Two simulations were performed, one with stimulation provided by the model predictive controller and one with no stimulation (the dashed trace that oscillates between higher and lower peaks). As can be seen from the traces in FIG. 55, model predictive controller stabilizes the Pa[CO₂] and prevents ventilatory oscillations leading to cycles of apneas and arousals. Resulting ventilation patterns for both cases are illustrated in FIG. 56, while FIG. 57 shows the stimulation pattern over a 10-minute simulation.

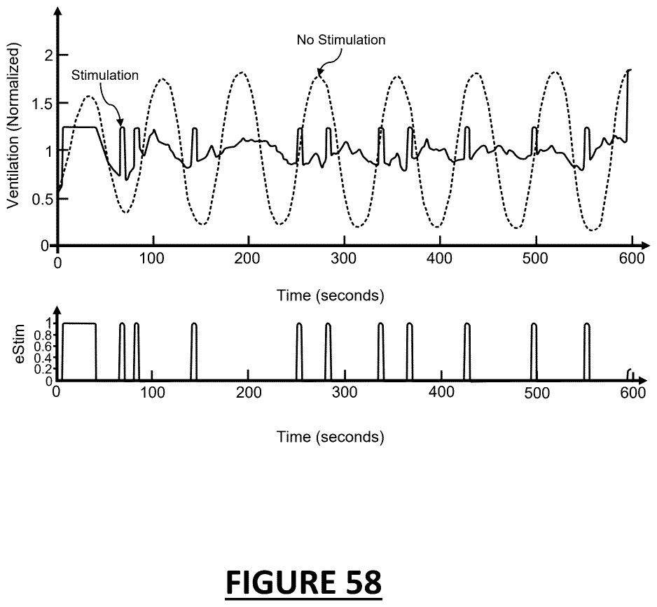

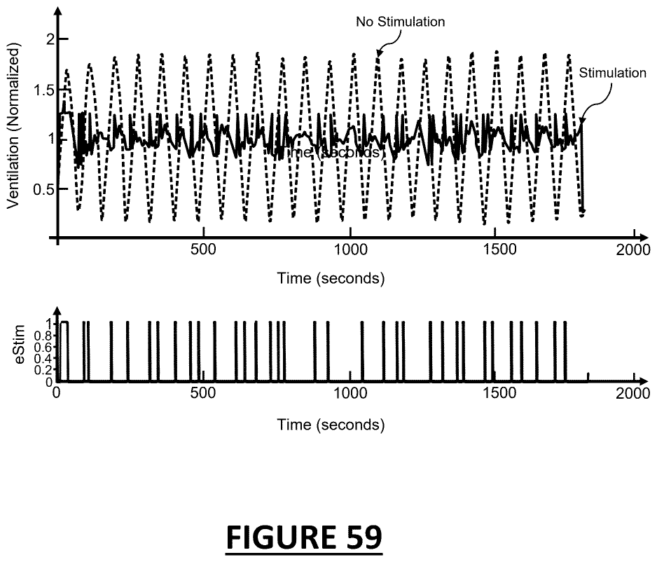

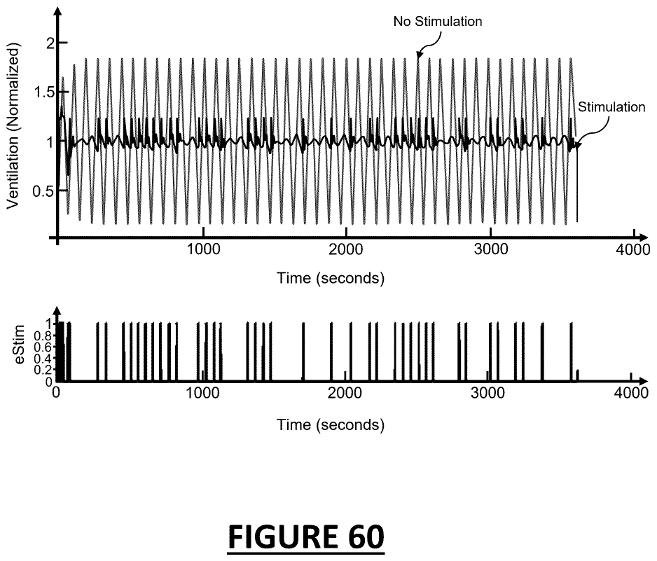

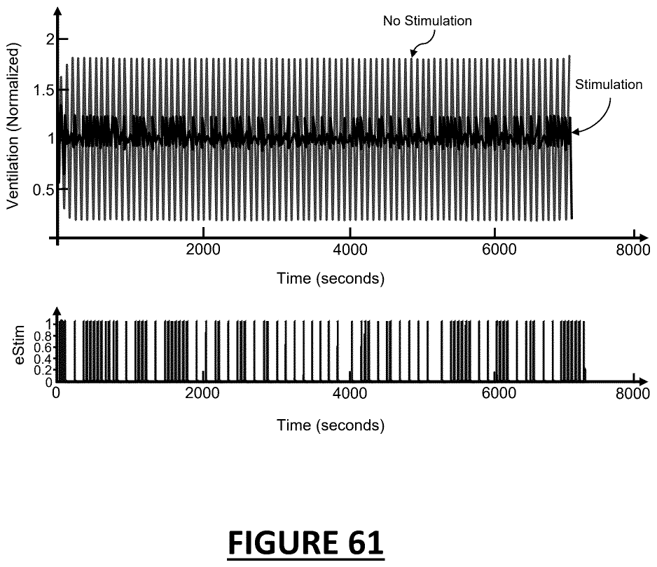

FIGS. 58, 59, 60 and 61 show the results from simulations done for 10, 30, 60 and 120 minutes respectively. FIG. 58 is a 10-minute stimulation with metabolic rate variability set at 25%. FIG. 59 is a 30-minute stimulation with metabolic rate variability set at 25%. FIG. 60 is a 60-minute stimulation with metabolic rate variability set at 5%. FIG. 61 is a 120-minute stimulation with metabolic rate variability set at 0%. As it can be seen from these figures (e.g., regardless of the duration of the simulations): 1) the model predictive controller maintains the Pa[CO₂] and prevents ventilatory oscillations leading to cycles of apneas and arousals, and 2) the optimal stimulation pattern does not have an obvious form that can be predicted without the use of the model predictive controller.

In certain examples, an implementation of the feedback control system may be provided. An example of such a system is shown in FIG. 62A with an overall system with the physiological model of patient's respiratory controller along with the feedback controller providing the therapy. FIG. 62B shows the desired change in the respiratory drive resulting in the utilization of the feedback controller, where the left side (6250) of the trace corresponds to no stimulation and right side (6252) to stimulation using, for example, the techniques discussed herein. Some options for the controller are: 1) Model Predictive Controller, 2) Classical controllers, such as PID controller, or 3) Logical controllers, such as a Short Demand Controller (discussed in more detail below).

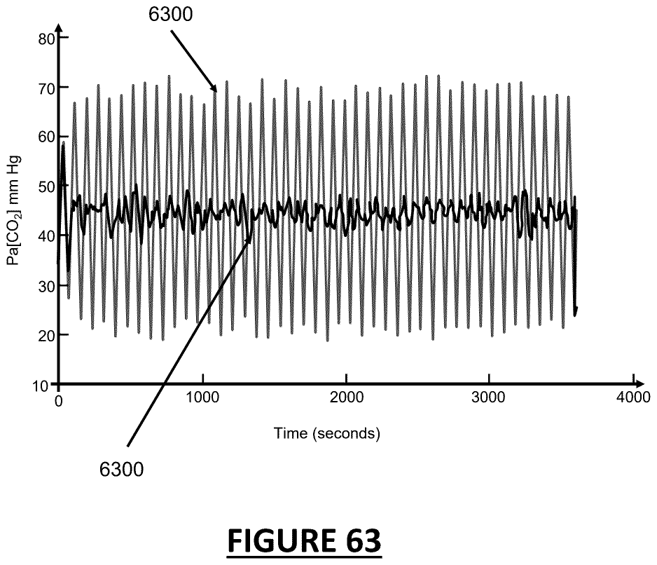

FIG. 63 illustrates the strong suppression of the oscillations in Pa[CO₂] achieved by the Model Predictive Controller where the standard deviation of the oscillations in Pa[CO₂] are reduced from 16 mm Hg (when no stim 6300 is applied) to 2.7 mm Hg (when stim 6302 is applied). The mean of no stim 6300 was 45 mm Hg and the mean of stim 6302 was 44 mm Hg.

### Short Demand Controller:

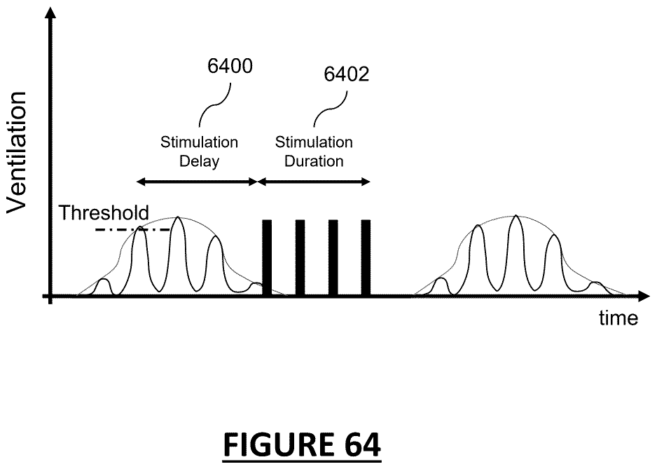

In other examples, a short demand controller may be used. In some examples, a short demand controller may be used instead of the model predictive controller. Using the short demand controller may provide improved sleep by using less suppression and/or a less complex algorithm. Operation of the Short Demand Controller is illustrated in the graph shown in FIG. 64. The Short Demand Controller monitors the ventilation, and once the Ventilation exceeds a threshold, a stimulation may be delivered with: 1) Stim Delay 6400, and 2) Stim Duration 6402.

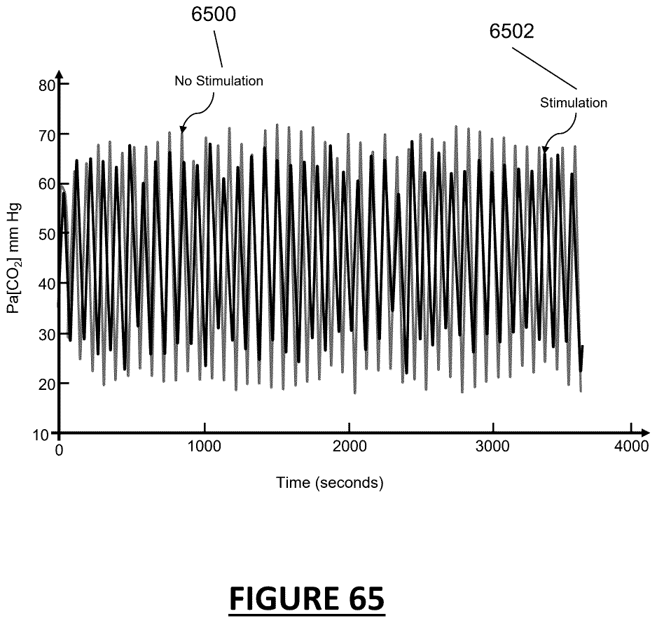

FIG. 65 shows the performance of the Short Demand Controller when the Stim Delay is set to 3 breaths and Stim Duration is set to 7 breaths. In that case, the Short Demand Controller reduces the variations (standard deviation) in Pa[CO₂] from 16 mm Hg (no stimulation 6500) to 11.3 mm Hg (stimulation 6502). In this example, a mean of 45 mm Hg for no stimulation and a mean of 45.4 mm Hg for stimulation were observed.

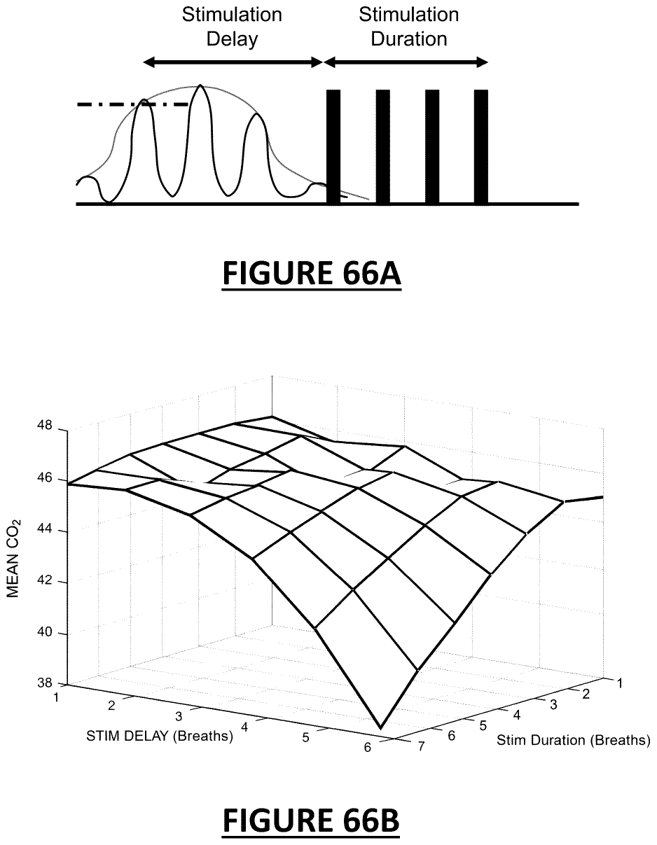

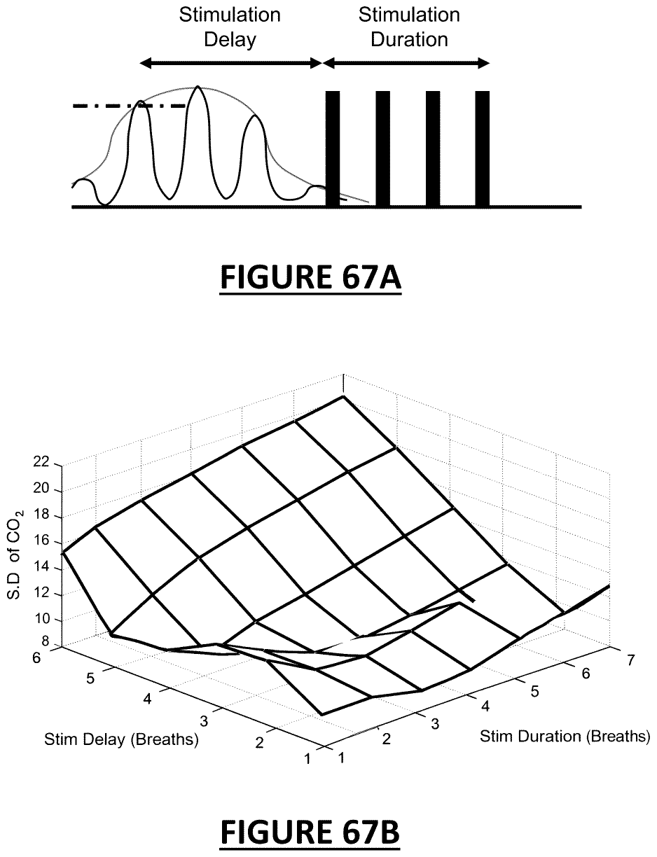

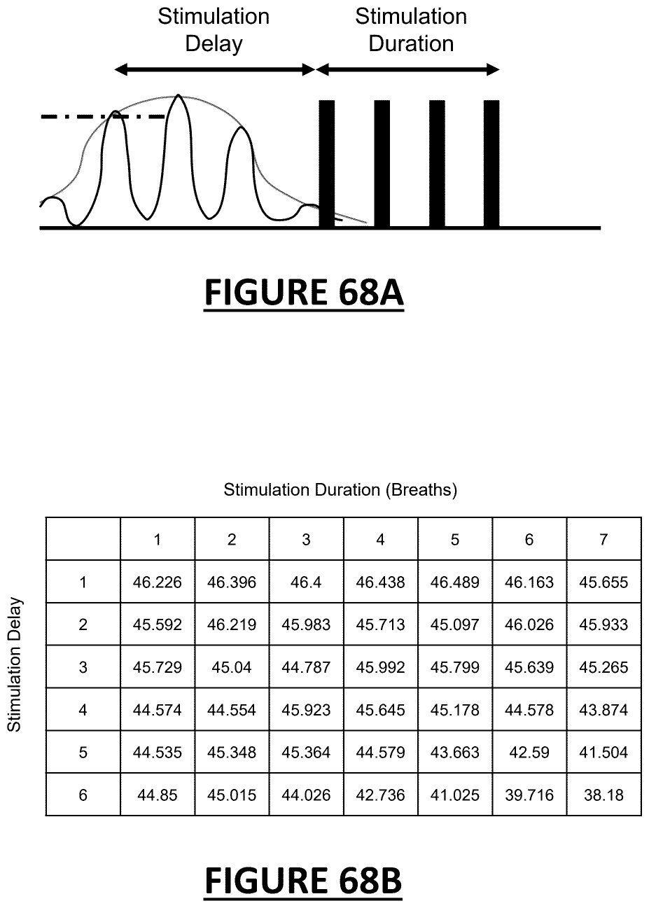

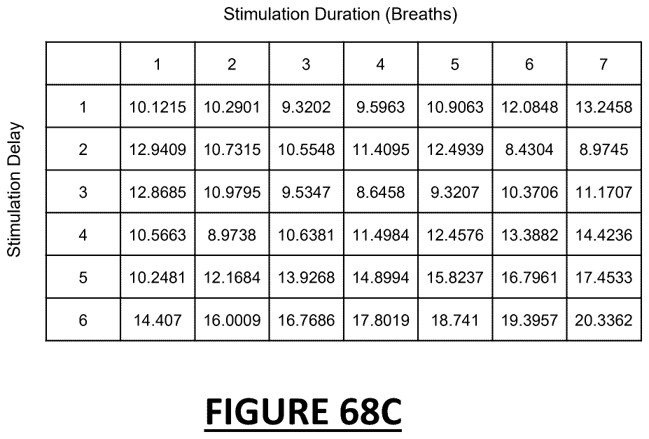

In order to determine the values (e.g., that are optimal) to use for Stim Delay and Stim Duration, an exhaustive search was carried out, results of which are illustrated in FIGS. 66A-68C, indicating that the minimum standard deviation is observed with Stim Duration of 6 breaths and Stim Delay of 2 breaths. FIGS. 66A and 66B illustrate exhaustive search based on the Mean of CO₂. FIGS. 67A and 67B illustrate exhaustive search based on the standard deviation of CO₂. FIGS. 68A-68C illustrate observations of using a short-demand controller with FIG. 68B show values for the mean CO₂ and FIG. 68C showing the standard deviation of CO₂.

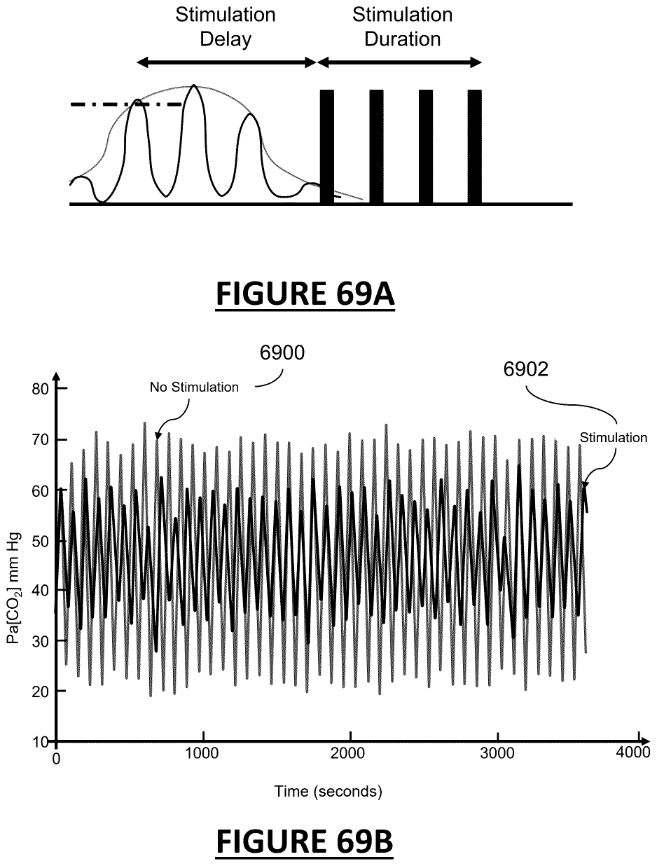

FIGS. 69A and 69B show the results of the Short Demand Controller. As shown in FIG. 69B, standard deviation of no stimulation 6900 was observed to be 16.3 mm Hg while the standard deviation with stimulation was 8.1 mm Hg. The mean with no stimulation was 45.3 mm Hg and the mean with stimulation was 46.2 mm Hg.

Results shown in FIG. 69B illustrated that using fixed values for its parameters, the Short Demand Controller was able to reduce the variability of Pa[CO₂] to half (e.g., as compared to the results shown in FIG. 65). In certain instances, improvements (e.g., additional improvements) may be achieved by: 1) the dynamic adjustments of the parameters—such as e.g., stim delay and stim duration, and 2) inclusion of additional parameter such as repeated stimulation and alternate stimulation.

### Examples Associated with Phenotype-Based Stimulation

Example 1: A method for determining a personalized stimulation pattern combination for a patient provided with sleep therapy, the method comprising: determining, from among a plurality of patient sleep phenotypes, a sleep phenotype of the patient; selecting, from a plurality of stimulation patterns, a personalized stimulation pattern based on the determined sleep phenotype; based on application of the personalized stimulation pattern to the patient while undergoing sleep therapy, receiving, from one or more sensors, sleep data of a patient; calculating, for the personalized stimulation pattern and based on the received sleep data of the patient, a respiration efficacy parameter for the patient.

Example 2. The method of Example 1, wherein the one or more sensors includes accelerometer, transthoracic impedance, pressure sensor, microphone, oxygen saturation sensor and/or a strain gauge. Example 3. The method of Example 1, wherein the respiration efficacy parameter includes tidal volume, minute ventilation, inspiratory duty cycle, airflow, oxygen saturation, oxygen desaturation index, apnea hypopnea index, and/or time below 90% oxygen saturation. Example 4. The method of Example 1, wherein each of the plurality of stimulation patterns includes one or more stimulation parameters, wherein the one or more stimulation parameters include amplitude, pulse width, frequency, rate, and/or duration. Example 5. The method of Example 1, wherein the plurality of stimulation patterns includes at least seven different stimulation patterns. Example 6. The method of Example 5, wherein a first stimulation pattern of the at least seven different stimulation pattern includes low stimulation amplitude, medium stimulation frequency, short stimulation duration, synchronization to the respiration waveform and one-to-one tracking with breathing. 7. The method of Example 6, wherein the low stimulation amplitude is between about 0.1 V and 1.5 V.

8. The method of Example 6, wherein the medium stimulation frequency is between about 10 and 30 Hz. 9. The method of Example 6, wherein the short stimulation duration is between about 100 and 500 ms. 10. The method of Example 6, wherein the first stimulation pattern invokes negative pressure reflex of the patient. 11. The method of Example 5, wherein a second stimulation pattern of the at least seven different stimulation pattern includes medium stimulation amplitude, medium stimulation frequency, long stimulation duration, synchronization to the respiration waveform and one-to-one tracking with breathing.

12. The method of Example 11, wherein the medium stimulation amplitude is between about 1.5 V and 4.5 V. 13. The method of Example 11, wherein the medium stimulation frequency is between about 10 and 30 Hz. 14. The method of Example 11, wherein the long stimulation duration is between about 3 and 5 seconds. 15. The method of Example 11, wherein the second stimulation pattern invokes caudal traction of the patient.

16. The method of Example 5, wherein a third stimulation pattern of the at least seven different stimulation pattern includes a high stimulation amplitude, high stimulation frequency, short stimulation duration, synchronization to the respiration waveform and one-to-one tracking with breathing. 17. The method of Example 16, wherein the high stimulation amplitude is between about 4.5 V and 10 V. 18. The method of Example 16, wherein the high stimulation frequency is between about 30 and 50 Hz. 19. The method of Example 16, wherein the short stimulation duration is between about 100 and 500 ms. 20. The method of Example 16, wherein the third stimulation pattern invokes afferent stimulation in the patient.

21. The method of Example 5, wherein a fourth stimulation pattern of the at least seven different stimulation pattern includes a very high stimulation amplitude, very low stimulation frequency and a continuous stimulation duration with no one-to-one tracking with the respiration waveform. 22. The method of Example 21, wherein the very high stimulation amplitude is between about 10.0 V and 15.0 V. 23. The method of Example 21, wherein the very low stimulation frequency is between about 0.1 and 1 Hz. 24. The method of Example 21, wherein the continuous stimulation duration is about 0.1 ms. 25. The method of Example 21, wherein the fourth stimulation pattern invokes afferent stimulation of the patient.

26. The method of Example 5, wherein a fifth stimulation pattern of the at least seven different stimulation pattern includes low, medium, and high stimulation amplitudes, medium frequency, medium stimulation duration, synchronization to the respiration waveform, and one-to-one tracking. 27. The method of Example 26, wherein the low stimulation amplitude is between about 0.1 V to 1.5 V. 28. The method of Example 26, wherein the medium stimulation amplitude is between about 1.5 V-4.5 V. 29. The method of Example 26, wherein the high stimulation amplitude is between about 4.5 V-10.0V. 30. The method of Example 26, wherein the medium stimulation frequency is between about 10-30 Hz. 31. The method of Example 26, wherein the medium duration between about 500 ms-3 s. 32. The method of Example 26, wherein the fifth stimulation pattern invokes entrainment. 33. The method of Example 26, wherein the phase may be adjusted.

34. The method of Example 5, wherein a sixth stimulation pattern of the at least seven different stimulation pattern includes a medium stimulation amplitude, medium stimulation frequency, medium stimulation duration, synchronization with the respiratory waveform, and 1-q tracking where q is maximized. 35. The method of Example 34, wherein the medium stimulation amplitude is about 1.5 V-4.5 V. 36. The method of Example 34, wherein the medium stimulation frequency is about 10-30 Hz. 37. The method of Example 34, wherein the medium stimulation duration is about 500 ms-3 s. 38. The method of Example 34, where q is greater than or equal to 2. 39. The method of Example 34, wherein the sixth stimulation pattern invokes neural plasticity.

40. The method of Example 5, wherein a seventh stimulation pattern of the at least seven different stimulation pattern includes a medium stimulation frequency, a very long stimulation duration, is not synchronized with the respiratory waveform, and has one-to-one tracking. 41. The method of Example 40, wherein the medium stimulation amplitude is between about 1-4.5 V. 42. The method of Example 40, wherein the medium stimulation frequency is about 10-30 Hz. 43. The method of Example 40, wherein the very long duration is about 5-7 s. 44. The method of Example 40, wherein the seventh stimulation includes a duty cycle of more than 50%. 45. The method of Example 1, wherein application of the personalized stimulation pattern to the patient while undergoing sleep therapy includes a series of unipolar or bipolar pulses applied as a burst. 46. The method of Example 45, wherein the burst includes a ramp up and ramp down phase. 47. The method of Example 46, wherein the ramp up may start from zero or a non-zero value. 48. The method of Example 46, wherein the ramp down may end at zero or a non-zero value. 49. The method of Example 45, wherein an amplitude of the burst is gradually raised. 50. The method of Example 49, wherein the gradually raised of the amplitude includes at least one of linear, non-linear, or step-wise.

51. The method of Example 1, further comprising determining, via a controller, arousals, unreliable sensor conditions, patient roll, and/or patient requests for start and stop of the therapy, and responds to these conditions. 52. The method of Example 1, further comprising determining, via a controller, a native respiratory rate and a simulation rate. 53. The method of Example 40, further comprising using, for a default stimulation mode in the absence of reliable sensor information, the seventh stimulation pattern. 54. The method of Example 5, wherein multiple ones of the plurality of stimulation patters are used when there is reliable sensor information and at least one is used based on determination of unreliable sensor information.

55. The method of Example 1, further comprising: storing calculated values for the respiration efficacy parameter for each of a plurality of different personalized stimulation patterns; and selecting, based on stored calculated values and for use in sleep therapy for the patient, one of a plurality of stimulation patterns.

56. The method of Example 26, wherein the fifth stimulation pattern is configured to start stimulation prior to the onset of patient inhalation and continue stimulation through the onset of the inhalation by the patient, and terminate prior to the end of the inhalation phase. 57. The method of Example 26, wherein the fifth stimulation pattern is configured to decrease a stimulation rate following determination of a reduction of airway occlusion and/or restoration of ventilation in the patient.

58. The method of Example 1, further comprising: based on determining that the calculated respiration efficacy parameter is less than a threshold value, determining another one of the plurality of patient sleep phenotypes. 59. The method of Example 1, further comprising: cycling through different ones of the plurality of patient sleep phenotypes during a single sleep session for the patient. 60. The method of Example 1, wherein each of the plurality of stimulation patterns includes multiple different parameters that are associated with sleep therapy provided to the patient.

61. The method of Example 1, wherein selection of the personalized stimulation pattern is automatically performed during a single sleep session of the patient. 62. The method of Example 1, wherein the method is performed over a single sleep session of the patient. 63. The method of Example 1, further comprising: selecting another personalized stimulation pattern based on the calculated respiration efficacy parameter. 64. The method of Example 63, wherein the selected other personalized stimulation pattern is further based a different one of the plurality of patient sleep phenotypes.

65. A sleep system comprising: A controller; A stimulator, wherein the sleep system is configured to perform the method of any one of Examples 1-64.

Examples Associated with Stimulation Through Inhalation

In this example, a method is used to deliver stimulation where the algorithm initiates the stimulation at an instance that is predicted to be a certain amount of time prior to the onset of inhalation, aka Approach, continuing to deliver the stimulation while waiting for the onset of inhalation, and after the onset of inhalation, delivering the stimulation for a short period of time before turning the stimulation off.

If the algorithm determines that the time duration between the onset of stimulation and the onset of the inhalation is getting longer, then the onset of stimulation is delayed in the subsequent stimulation cycles.

200. A method of controlling a respiratory sleep device to provide respiratory sleep therapy to a sleeping patient, the method comprising: receiving, from one or more sensors, sleep data of the patient; determining, based on the sleep data, an inhalation approach instance that is before onset of inhalation by the patient; based on determination of the inhalation approach instance, causing electrical stimulation to be delivered to a phrenic nerve of the patient prior to the onset of inhalation by the patient; continuing to cause the electrical stimulation to be delivered to the phrenic nerve while waiting for onset of inhalation by the patient; after the onset of inhalation, continuing to cause the electrical stimulation to be delivered until a post onset inhalation instance is determined, wherein the post onset inhalation instance occurs after onset of inhalation by the patient; and based on expiration of the post inhalation amount of time, causing the electronical stimulation to be turned off.

201. The method of Example 200, wherein determination of the inhalation approach instance includes executing a prediction process to predict when the onset of inhalation by the patient will occur.

202. The method of Example 200, wherein the post onset inhalation instance is determined based on expiration of a predetermined amount of time after onset of inhalation.

203. The method of Example 200, wherein the post onset inhalation instance is determined based on determination of exhalation by the patient.

204. A method of controlling a respiratory sleep device to provide respiratory sleep therapy to a sleeping patient, the method comprising: receiving, from one or more sensors, sleep data of the patient; determining, based on the sleep data, an approach instance that is at a pre-inhalation time before onset of an instance of inhalation by the patient; determining a post onset inhalation time that is after onset of the instance of inhalation by the patient; causing electrical stimulation to be continuously delivered to a phrenic nerve of the patient from the pre-inhalation time to the post onset inhalation time; and causing the electrical stimulation to be switched off after the post onset inhalation time and prior to another instance of inhalation by the patient.

205. The method of Example 204, further comprising predicting, based on the sleep data, an inhalation event, wherein the determination of the approach instance is further based on predication of the inhalation event.

206. The method of any one of Examples 200-205, wherein the approach instance is in a range of about 0.1 to 0.5 seconds prior to onset of inhalation by the patient, and a total stimulation duration from the approach instant until turned off is between about 0.7 seconds to 1.2 seconds.

Examples Associated with Decrease Stim Rate

301. A method of controlling a respiratory sleep device to provide respiratory sleep therapy to a sleeping patient, the method comprising: causing, via an implanted device, electrical stimulation therapy to be delivered to a target nerve of a patient; receiving, from one or more sensors, sensor data in connection with the sleeping patient; generating a sensor reliability determination based on the sensor data and/or absence of the sensor data, the sensor reliability determination being between at least reliable and unreliable; based on sensor reliability determination being reliable, determining ventilation efficacy based on the sensor data; based on the ventilation efficacy being above a threshold level, withholding electrical stimulation therapy; based on the ventilation efficacy being below the threshold level, delivering electrical stimulation therapy for a first period of time, and after the first period of time, halting the electrical stimulation therapy; based on sensor reliability determination being unreliable, delivering electrical stimulation therapy for a second period of time, and after the second period of time, halting the electrical stimulation therapy.

302. The method of Example 301, wherein the first and second period of time are the same length of time.

303. The method of Example 301, wherein the first and second period of time are different lengths of time.

310. A method of controlling a respiratory sleep device to provide respiratory sleep therapy to a sleeping patient, the method comprising:
receiving, from one or more sensors, sensor data in connection with the sleeping patient;
determining a breathing rate of the sleeping patient;
initiating, via an implanted device, electrical stimulation therapy to be delivered to a target nerve of a patient at a first rate that is less than the breathing rate of the sleeping patient;
tracking an amount of time that has elapsed since initiating electrical stimulation therapy; and
based on the tracked amount of time exceeding a first threshold, decreasing a rate of the electrical stimulation therapy to a second rate that is less than the first rate.

311. The method of Example 310, further comprising: generating a sensor reliability determination based on the sensor data and/or absence of the sensor data, the sensor reliability determination being between at least reliable and unreliable,
wherein decreasing the rate of the electrical stimulation therapy to the second rate is additional based on the sensor reliability determination being not reliable.

312. The method of Example 310, wherein the tracked amount of time is at least about 20 seconds. 313. The method of Example 312, wherein the tracked about of time is between about 20 and 40 seconds. 314. The method of Example 310, wherein the first threshold is dynamically determined, for example, based on sensor or real-time data from the sleeping patient. 315. The method of Example 310, wherein the first threshold is a fixed time. 316. The method of Example 310, further comprising: based on detection of an apnea, triggering the initiation of the electrical stimulation therapy. 317. The method of claim 310, wherein the first rate is about 1 per minute less than the breathing rate of the sleeping patient.

320. The method of any of Examples 300-320, wherein the sensor reliability determination is unreliable based on any one of: 1) at least one sensor has absolute value out of range; 2) at least one sensor has peak-to-peak value out of range; 3) at least one sensor has detected respiratory rate out of range; 4) respiratory rates detected by at least two sensors differ more than 1 breaths per minute; 5) based on an impedance sensor detecting respiration, but an accelerometer not detecting respiration.

Examples Associated with Fixed/Default/Main Stimulation Delivery

In some examples, a method is provided to deliver stimulation with a fixed duration, at a fixed rate where the stimulation rate coincides with the natural respiratory rate of the patient and the desired phase of the stimulation is such that the stimulation begins prior to the onset of inhalation, stimulation continues through the onset of the inhalation and the stimulation ends before the inhalation ends, e.g., the onset of inhalation occurs when the stimulation is on.

If the algorithm notices that the condition described above is not being met, then the stimulation rate and or duration are adjusted: 1) If the inhalation begun before the stimulation begun, then the stimulation rate is increased. 2) If the stimulated ended after the inhalation ended, then the stimulation duration is reduced.

400. A method of controlling a respiratory sleep device to provide respiratory sleep therapy to a sleeping patient, the method comprising: causing, via an implanted device, electrical stimulation therapy to be delivered to a target nerve of a patient; wherein the electrical stimulation therapy is delivered for a fixed duration and at a fixed rate, wherein a stimulation rate coincides with a natural respiratory rate of the patient, wherein a desired phase of the electrical stimulation is such that stimulation begins prior to the onset of inhalation by the patient, wherein stimulation continues through onset of the inhalation and stimulation ends before the inhalation ends; and monitoring one or more parameters of the electrical stimulation therapy and adjusting as needed.

401. The method of Example 400, wherein based on determination that inhalation began before stimulation, then the stimulation rate is increased. 402. The method of claim 400, wherein based on determination that stimulation ended after inhalation ended, then the stimulation duration is reduced.

Any or all of the methods, processes, or other steps described herein may be implemented by a processor. Multiple embodiments are discussed herein, and it should be understood that modifications, substitutions, and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both, unless the disclosure states otherwise. Furthermore, characteristics or steps which have been described may also be used in combination with other characteristics or steps and in any order unless the disclosure or context suggests otherwise.

1. A method to formulate stimuli to be delivered to the phrenic nerve for the treatment of central and/or mixed sleep apneas, comprising:
monitoring respiration using a detector of ventilation;
computing stimulation parameters using a predictive algorithm that is designed to minimize the oscillations in the ventilatory drive of the patient;
generating and delivering stimuli to the phrenic nerve using a stimulator to augment the ventilation and to modulate the ventilatory drive; and
modifying the stimulation parameters using a feedback algorithm to minimize the oscillations in the ventilatory drive of the patient.

2. The method of claim 1, wherein the detector of ventilation is one or more of a transthoracic impedance sensor, and an accelerometer.

3. The method of claim 1, wherein the predictor is one of Model Predictive Controller or Short Demand Controller.

4. The method of claim 1, wherein the feedback algorithm is minimum squared error from one of nominal ventilation or estimated Pa[CO₂].

5. The method of claim 2, wherein monitoring respiration comprises:
concurrently acquiring transthoracic impedance and multi-axis accelerometer signals; and
fusing them to generate a digital respiratory cycle signal.

6. The method of claim 3, wherein the Model Predictive Controller sets an observation horizon equal to twice a measured circulatory delay of the patient.

7. The method of claim 1, wherein generating and delivering stimuli comprises applying biphasic pulses to the phrenic nerve.

8. The method of claim 1, wherein the stimulator delivers stimuli at a rate that is less than a native respiratory rate determined by the detector.

9. The method of claim 8, wherein the stimulator delivers stimuli at a rate that is approximately one breath per minute less than the native respiratory rate determined by the detector.

10. The method of claim 1, wherein delivering the stimuli begins at an approach time in the range of 0.1 to 0.5 seconds prior to the onset of inhalation detected by the detector.
